(19) United States
(12) Patent Application Publication
(10) Pub. No.: US 2023/0340581 A1
(43) Pub. Date: Oct. 26, 2023

(54) NON-EXTENSIBLE OLIGONUCLEOTIDES IN DNA AMPLIFICATION REACTIONS

(71) Applicant: William Marsh Rice University, Houston, TX (US)

(72) Inventors: David ZHANG, Houston, TX (US); Kerou ZHANG, Houston, TX (US)

(73) Assignee: William Marsh Rice University, Houston, TX (US)

(21) Appl. No.: 17/999,960

(22) PCT Filed: May 27, 2021
(86) PCT No.: PCT/US2021/034524

§ 371 Date: Nov. 28, 2022

Related U.S. Application Data
(60) Provisional application No. 63/155,398, filed on Mar. 2, 2021.
(60) Provisional application No. 63/030,452, filed on May 27, 2020.

(51) Int. Cl. C12Q 1/6858; C12Q 1/6869

(52) U.S. Cl. CPC C12Q 1/6858; C12Q 1/6869; C12Q 1/6806; C12Q 2600/166

(57) ABSTRACT

Provided herein are non-extensible oligonucleotides for suppressing enzymatic extension through rationally designed secondary structures at the 3′ end. Embodiments of the invention include procedures for integration with real-time polymerase chain reaction, blocker displacement amplification in quantitative PCR, next generation sequencing (NGS), and long-read sequencing.

### REFERENCE TO RELATED APPLICATIONS

The present application claims the priority benefit of U.S. Provisional Application No. 63/155,398, filed Mar. 2, 2021, and U.S. Provisional Application No. 63/030,452, filed May 27, 2020, the entire contents of each of which is incorporated herein by reference.

### STATEMENT REGARDING FEDERALLY SPONSORED RESEARCH

This invention was made with government support under Grant No. R01CA203964 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on May 18, 2021, is named RICEP0076WO_ST25.txt and is 55.3 kilobytes in size.

### BACKGROUND

The development of this disclosure was funded in part by the Cancer Prevention and Research Institute of Texas (CPRIT) under Grant No. RP180147.

### 1. Field

The present invention relates generally to the field of molecular biology. More particularly, it concerns compositions comprising and methods of using non-extensible oligonucleotides (NEOs) with rationally designed secondary structures at or near their 3′ end, which are not extended enzymatically by high fidelity DNA polymerases with 3′->5′ exonuclease activity.

### 2. Description of Related Art

DNA polymerases are used in a variety of applications to extend DNA oligonucleotides. However, there are certain cases where it is desirable for some DNA oligonucleotides in a solution containing DNA polymerase not to be enzymatically extended. Historically, researchers have used DNA oligonucleotides with chemical modifications at the 3′ end to prevent enzymatic extension; these modifications include inverted DNA nucleotides, poly-ethylene glycol spacers, alkane-based spacers, fluorophores, quenchers, minor-groove binders, and others. These chemical modifications are expensive to functionalize to DNA oligonucleotides after oligonucleotide synthesis, significantly reduce the purity and yield of the oligonucleotide, and significantly extend the synthesis turnaround time. For these reasons, non-extensible oligonucleotides that are not chemically modified are needed.

### SUMMARY

As such, provided herein are non-extensible oligonucleotides (NEO) that suppress enzymatic extension through rationally designed secondary structures at the 3′ end of the NEO.

In one embodiment, provided herein are compositions comprising a DNA template, a DNA polymerase, and a non-extensible oligonucleotide, wherein the DNA template comprises continuously from 5′ to 3′ an upstream sequence and a probe binding sequence, wherein the non-extensible oligonucleotide comprises from 5′ to 3′: a binding sequence that is at least 70% identical to the reverse complement of the probe binding sequence of the DNA template, and a terminator hairpin, positioned at the 3′-end of the non-extensible oligonucleotide, that comprises: a first stem sequence, a second stem sequence, wherein the second stem sequence is the reverse complement of the first stem sequence, and a loop sequence positioned between the first stem sequence and the second stem sequence. For example, the binding sequence is at least 70%, 75%, 80%, 85%, 90%, 95%, 96%, 97%, 98%, or 99% identical to the reverse complement of the probe binding sequence of the DNA template.

In some aspects, the binding sequence of the non-extensible oligonucleotide is between 10 and 300 nucleotides long. For example, the binding sequence of the non-extensible oligonucleotide may be 10-300, 10-250, 10-200, 10-150, 10-100, 10-90, 10-80, 10-70, 10-60, 10-50, 10-45, 10-40, 10-35, 10-30, 15-30, 15-35, 15-40, 15-45, 15-50, 20-35, 20-40, 20-45, or 20-50 nucleotides long. For example, the binding sequence of the non-extensible oligonucleotide may be at least or about 10, 15, 20, 25, 30, 35, 40, 45, 50, 55, 60, 65, 70, 75, 80, 85, 90, 95, 100, 120, 140, 160, 180, 200, 220, 240, 260, 280, or 300 nucleotides long.

In some aspects, the terminator hairpin of the non-extensible oligonucleotide is not the reverse complement of the upstream sequence of the DNA template. In some aspects, the terminator hairpin of the non-extensible oligonucleotide is unable to hybridize to the upstream sequence of the DNA template.

In some aspects, the first stem sequence of the terminator hairpin is between 3 and 8 nucleotides long. For example, the first stem sequence of the terminator hairpin may be at least or about 3, 4, 5, 6, 7, or 8 nucleotides long. In some aspects, the first stem sequence of the terminator hairpin is four nucleotides long. In some aspects, the second stem sequence of the terminator hairpin is between 3 and 8 nucleotides long. For example, the second stem sequence of the terminator hairpin may be at least or about 3, 4, 5, 6, 7, or 8 nucleotides long. In some aspects, the second stem sequence of the terminator hairpin is four nucleotides long. In some aspects, the first stem sequence and the second stem sequence of the terminator hairpin are both four nucleotides long.

In some aspects, the terminator hairpin has an adenine nucleotide as its 3′-most nucleotide. In some aspects, the first stem sequence is 5′-TCTC-3′ and the second stem sequence is 5′-GAGA-3′. In some aspects, the first stem sequence is 5′-GTTC-3′ and the second stem sequence is 5′-GAAC-3′.

In some aspects, the loop sequence of the terminator hairpin is between 3 and 10 nucleotides long. For example, the loop sequence of the terminator hairpin may be at least or about 3, 4, 5, 6, 7, 8, 9, or 10 nucleotides long. In some aspects, the loop sequence of the terminator hairpin is four nucleotides long. In some aspects, the loop sequence is 5′-GCAA-3′.

In some aspects, the non-extensible oligonucleotide further comprises a middle hairpin positioned between the binding sequence and the terminator hairpin. The middle hairpin may comprise a third stem sequence, a fourth stem sequence, wherein the fourth stem sequence is the reverse complement of the third stem sequence, and a second loop sequence positioned between the third stem sequence and the fourth stem sequence. In some aspects, the 3′-most nucleotide of the terminator hairpin is a cytosine. In some aspects, the first stem sequence of the terminator hairpin and the second stem sequence of the terminator hairpin are each between 3 and 8 nucleotides long. For example, the first stem sequence and the second stem sequence of the terminator hairpin may each be, independently, at least or about 3, 4, 5, 6, 7, or 8 nucleotides long. In some aspects, the first stem sequence is 5′-GTTA-3′ and the second stem sequence is 5′-TAAC-3′. In some aspects, the first stem sequence is 5′-GATT-3′ and the second stem sequence is 5′-AATC-3′. In some aspects, the first loop sequence of the terminator hairpin is between 3 and 10 nucleotides long. For example, the first loop sequence may be at least or about 3, 4, 5, 6, 7, 8, 9, or 10 nucleotides long. In some aspects, the first loop sequence is 5′-GCAA-3′. In some aspects, the third stem sequence of the middle hairpin and the fourth stem sequence of the middle hairpin are each between 3 and 20 nucleotides long. For example, the third stem sequence and the fourth stem sequence of the middle hairpin may each be, independently, at least or about 3, 4, 5, 6, 7, 8, 9, 10, 11, 12, 13, 14, 15, 16, 17, 17, 19, or 20 nucleotides long. In some aspects, third stem sequence is 5′-GAGAAC-3′ and the fourth stem sequence is 5′-GTTCTC-3′. In some aspects, the third stem sequence is 5′-CCTGTA-3′ and the fourth stem sequence is 5′-TACAGG-3′. In some aspects, the second loop sequence of the middle hairpin is between 3 and 15 nucleotides long. For example, the second loop sequence of the middle hairpin may be at least or about 3, 4, 5, 6, 7, 8, 9, 10, 11, 12, 13, 14, or 15, nucleotides long. In some aspects, the second loop sequence of the middle hairpin is 5′-ATTA-3′. In some aspects, the second loop sequence of the middle hairpin is 5′-CACA-3′. In certain aspects, the first stem sequence is 5′-GTTA-3′, the second stem sequence is 5′-TAAC-3′, the first loop sequence is 5′-GCAA-3′, third stem sequence is 5′-GAGAAC-3′, the fourth stem sequence is 5′-GTTCTC-3′, and the second loop sequence of the middle hairpin is 5′-ATTA-3′. In certain aspects, the first stem sequence is 5′-GATT-3′, the second stem sequence is 5′-AATC-3′, the first loop sequence is 5′-GCAA-3′, third stem sequence is 5′-GAGAAC-3′, the fourth stem sequence is 5′-GTTCTC-3′, and the second loop sequence of the middle hairpin is 5′-ATTA-3′. In certain aspects, the first stem sequence is 5′-GTTA-3′, the second stem sequence is 5′-TAAC-3′, the first loop sequence is 5′-GCAA-3′, third stem sequence is 5′-CCTGTA-3′, the fourth stem sequence is 5′-GGACAT-3′, and the second loop sequence of the middle hairpin is 5′-CACA-3′.

In some aspects, the non-extensible oligonucleotide further comprises a mismatch sequence positioned between the binding sequence and the terminator hairpin. In some aspects, the mismatch sequence is between 1 and 100 nucleotides long. For example, the mismatch sequence may be between 1-100, 1-90, 1-80, 1-70, 1-60, 1-50, 1-40, 1-30, 1-25, 1-20, 1-15, 1-10, 5-10, 5-15, 5-20, 10-20, 10-25, or 10-30 nucleotides long. For example, the mismatch sequence may be at least or about 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides long. In some aspects, the mismatch sequence is at most 30% identical to the reverse complement of the upstream sequence of the DNA template. For example, the mismatch sequence may be at most or about 5%, 10%, 15%, 20%, 25%, or 30% identical to the reverse complement of the upstream sequence of the DNA template. In some aspects, the mismatch sequence is unable to hybridize to the upstream sequence of the DNA template.

In some aspects, the mismatch sequence does not form a non-linear secondary structure. In some aspects, no two subsequences of the mismatch sequence for an intramolecular structure stronger than -2 kcal/mol. In some aspects, the mismatch sequence does not form a hairpin. In some aspects, the mismatch sequences is between 5 and 20 nucleotides long. For example, the mismatch sequence is at least or about 5, 6, 7, 8, 9, 10, 11, 12, 13, 14, 15, 16, 17, 18, 19, or 20 nucleotides long.

In some aspects, the mismatch sequence comprises a former subsequence and a latter subsequence, wherein the latter subsequence is the reverse complement of the former subsequence. In some aspects, the former subsequence and the latter subsequence are each at least four nucleotides long. In some aspects, the former subsequence and the latter subsequence are each six nucleotides long. In some aspects, the mismatch sequence comprises a plurality of former subsequences and a plurality of latter subsequences, wherein each former subsequence is the reverse complement of a corresponding latter subsequence. In some aspects, each former subsequence and each latter subsequence is at least four nucleotides long.

In some aspects, the mismatch sequence comprises, from 5′ to 3′, a first subsequence, a second subsequence, a third subsequence, and a fourth subsequence, wherein the first subsequence is the reverse complement of the second subsequence, and wherein the third subsequence is the reverse complement of the fourth subsequence. In some aspects, each of the first subsequence, the second subsequence, the third subsequence, and the fourth subsequence are between four and 15 nucleotides long. For example, each of the first subsequence, the second subsequence, the third subsequence, and the fourth subsequence may be at least or about 4, 5, 6, 7, 8, 9, 10, 11, 12, 13, 14, or 15 nucleotides long.

In some aspects, the mismatch sequence comprises, from 5′ to 3′, a first subsequence, a second subsequence, a third subsequence, and a fourth subsequence, wherein the first subsequence is the reverse complement of the fourth subsequence, and wherein the second subsequence is the reverse complement of the third subsequence. In some aspects, each of the first subsequence, the second subsequence, the third subsequence, and the fourth subsequence are between four and 15 nucleotides long. For example, each of the first subsequence, the second subsequence, the third subsequence, and the fourth subsequence may be at least or about 4, 5, 6, 7, 8, 9, 10, 11, 12, 13, 14, or 15 nucleotides long. In some aspects, a non-complementary region is positioned between the double stranded region formed by the first subsequence and fourth subsequence and the double stranded region formed by the second subsequence and the third subsequence. In some aspects, the non-complementary region is between 3 and 10 nucleotides long. For example, the non-complementary region may be at least or about 3, 4, 5, 6, 7, 8, 9, or 10 nucleotides long.

In some aspects, the non-extensible oligonucleotide does not comprise an artificial chemical modification or a non-natural DNA nucleotide at its 3′ end.

In some aspects, the upstream sequence of the DNA template is between 3 and 100 nucleotides long. For example, the upstream sequence of the DNA template may be between 3-100, 3-90, 3-80, 3-70, 3-60, 3-50, 3-40, 3-30, 3-25, 3-20, 5-15, 5-20, 5-25, 5-30, 5-35, 10-20, 10-25, or 10-30 nucleotides long. For example, the upstream sequence of the DNA template may be at least or about 3, 4, 5, 6, 7, 8, 9, 10, 15, 20, 25, 30, 35, 40, 45, 50, 55, 60, 65, 70, 75, 80, 85, 90, 95, or 100 nucleotides long.

In some aspects, the probe binding sequence of the DNA template is between 10 and 300 nucleotides long. For example, the probe binding sequence of the DNA template may be between 10-300, 10-250, 10-200, 10-150, 10-100, 10-90, 10-80, 10-70, 10-60, 10-50, 10-45, 10-40, 10-35, 10-30, 15-30, 15-35, 15-40, 15-45, 15-50, 20-35, 20-40, 20-45, or 20-50 nucleotides long. For example, the probe binding sequence of the DNA template may be at least or about 10, 15, 20, 25, 30, 35, 40, 45, 50, 55, 60, 65, 70, 75, 80, 85, 90, 95, 100, 120, 140, 160, 180, 200, 220, 240, 260, 280, or 300 nucleotides long.

In some aspects, the DNA polymerase is a high-fidelity DNA polymerase with 3′ to 5′ exonuclease activity.

In some aspects, the composition may comprise a population of non-extensible oligonucleotides and a population of DNA templates, wherein various non-extensible oligonucleotides of the population have different binding sequences that are at least 70% identical to the reverse complements of various probe binding sequences found within the population of DNA templates. For example, the composition may comprise at least or about 2, 3, 4, 5, 6, 7, 8, 9, 10, 11, 12, 13, 14, 15, or 20 different non-extensible oligonucleotides.

In one embodiment, provided herein are compositions comprising a DNA template, a DNA polymerase, and a non-extensible oligonucleotide, wherein the DNA template comprises continuously from 5′ to 3′ an upstream sequence and a probe binding sequence, wherein the non-extensible oligonucleotide comprises from 5′ to 3′: a binding sequence that is at least 70% identical to the reverse complement of the probe binding sequence of the DNA template, a mismatch sequence comprising: a first stem sequence, and a second stem sequence, wherein the second stem sequence is the reverse complement of the first stem sequence, and a tail sequence that is at most 40% identical to the reverse complement of the upstream sequence of the DNA template. For example, the binding sequence is at least 70%, 75%, 80%, 85%, 90%, 95%, 96%, 97%, 98%, or 99% identical to the reverse complement of the probe binding sequence of the DNA template. For example, the tail sequence is at most or about 5%, 10%, 15%, 20%, 25%, 30%, 35%, or 40% identical to the reverse complement of the upstream sequence of the DNA template.

In some aspects, the binding sequence of the non-extensible oligonucleotide is between 10 and 300 nucleotides long. For example, the binding sequence of the non-extensible oligonucleotide may be 10-300, 10-250, 10-200, 10-150, 10-100, 10-90, 10-80, 10-70, 10-60, 10-50, 10-45, 10-40, 10-35, 10-30, 15-30, 15-35, 15-40, 15-45, 15-50, 20-35, 20-40, 20-45, or 20-50 nucleotides long. For example, the binding sequence of the non-extensible oligonucleotide may be at least or about 10, 15, 20, 25, 30, 35, 40, 45, 50, 55, 60, 65, 70, 75, 80, 85, 90, 95, 100, 120, 140, 160, 180, 200, 220, 240, 260, 280, or 300 nucleotides long.

In some aspects, the mismatch sequence of the non-extensible oligonucleotide is between 10 and 100 nucleotides long. For example, the mismatch sequence may be between 10-100, 10-90, 10-80, 10-70, 10-60, 10-50, 10-40, 10-30, 10-25, or 10-20 nucleotides long. For example, the mismatch sequence may be at least or about 10, 11, 12, 13, 14, 15, 16, 17, 18, 19, 20, 25, 30, 35, 40, 45, 50, 55, 60, 65, 70, 75, 80, 85, 90, 95, or 100 nucleotides long.

In some aspects, the first stem sequence of the mismatch sequence is between 4 and 45 nucleotides long. For example, the first stem sequence of the mismatch sequence may be between 4-45, 4-40, 4-35, 4-30, 4-25, 4-20, 4-15, 4-10, 8-45, 8-40, 8-35, 8-30, 8-25, 8-20, or 8-15 nucleotides long. For example, the first stem sequence of the mismatch sequence may be at least or about 4, 5, 6, 7, 8, 9, 10, 11, 2, 13, 14, 15, 20, 25, 30, 35, 40, or 45 nucleotides long. In some aspects, the second stem sequence of the mismatch sequence is between 4 and 45 nucleotides long. For example, the second stem sequence of the mismatch sequence may be between 4-45, 4-40, 4-35, 4-30, 4-25, 4-20, 4-15, 4-10, 8-45, 8-40, 8-35, 8-30, 8-25, 8-20, or 8-15 nucleotides long. For example, the second stem sequence of the mismatch sequence may be at least or about 4, 5, 6, 7, 8, 9, 10, 11, 2, 13, 14, 15, 20, 25, 30, 35, 40, or 45 nucleotides long.

In some aspects, the mismatch sequence comprises a plurality of first stem sequence and a plurality of second stem sequences, wherein each second stem sequence is the reverse complement of a corresponding first stem sequence. In some aspects, each first stem sequence and each second stem sequence is between four and 45 nucleotides long. For example, each first stem sequence and each second stem sequence may be between 4-45, 4-40, 4-35, 4-30, 4-25, 4-20, 4-15, 4-10, 8-45, 8-40, 8-35, 8-30, 8-25, 8-20, or 8-15 nucleotides long. For example, each first stem sequence and each second stem sequence may be at least or about 4, 5, 6, 7, 8, 9, 10, 11, 2, 13, 14, 15, 20, 25, 30, 35, 40, or 45 nucleotides long.

In some aspects, the mismatch sequence comprises, from 5′ to 3′, a first subsequence, a second subsequence, a third subsequence, and a fourth subsequence, wherein the first subsequence is the reverse complement of the second subsequence, and wherein the third subsequence is the reverse complement of the fourth subsequence. In some aspects, each of the first subsequence, the second subsequence, the third subsequence, and the fourth subsequence are between four and 15 nucleotides long. For example, each of the first subsequence, the second subsequence, the third subsequence, and the fourth subsequence may be at least or about 4, 5, 6, 7, 8, 9, 10, 11, 12, 13, 14, or 15 nucleotides long.

In some aspects, the mismatch sequence comprises, from 5′ to 3′, a first subsequence, a second subsequence, a third subsequence, and a fourth subsequence, wherein the first subsequence is the reverse complement of the fourth subsequence, and wherein the second subsequence is the reverse complement of the third subsequence. In some aspects, each of the first subsequence, the second subsequence, the third subsequence, and the fourth subsequence are between four and 15 nucleotides long. For example, each of the first subsequence, the second subsequence, the third subsequence, and the fourth subsequence may be at least or about 4, 5, 6, 7, 8, 9, 10, 11, 12, 13, 14, or 15 nucleotides long. In some aspects, a non-complementary region is positioned between the double stranded region formed by the first subsequence and fourth subsequence and the double stranded region formed by the second subsequence and the third subsequence. In some aspects, the non-complementary region is between 3 and 10 nucleotides long. For example, the non-complementary region may be at least or about 3, 4, 5, 6, 7, 8, 9, or 10 nucleotides long.

In some aspects, the tail sequence is between 3 and 15 nucleotides long. For example, the tail sequence may be at least or about 3, 4, 5, 6, 7, 8, 19, 10, 11, 12, 13, 14, or 15 nucleotides long. In some aspects, the tail sequence of the non-extensible oligonucleotide is unable to hybridize to the upstream sequence of the DNA template. In some aspects, the tail sequence of the non-extensible oligonucleotide does not form a non-linear secondary structure. In some aspects, the tail sequence of the non-extensible oligonucleotide does not form a hairpin.

In some aspects, the non-extensible oligonucleotide does not comprise an artificial chemical modification or a non-natural DNA nucleotide at its 3′ end.

In some aspects, the upstream sequence of the DNA template is between 3 and 100 nucleotides long. For example, the upstream sequence of the DNA template may be between 3-100, 3-90, 3-80, 3-70, 3-60, 3-50, 3-40, 3-30, 3-25, 3-20, 5-15, 5-20, 5-25, 5-30, 5-35, 10-20, 10-25, or 10-30 nucleotides long. For example, the upstream sequence of the DNA template may be at least or about 3, 4, 5, 6, 7, 8, 9, 10, 15, 20, 25, 30, 35, 40, 45, 50, 55, 60, 65, 70, 75, 80, 85, 90, 95, or 100 nucleotides long.

In some aspects, the probe binding sequence of the DNA template is between 10 and 300 nucleotides long. For example, the probe binding sequence of the DNA template may be between 10-300, 10-250, 10-200, 10-150, 10-100, 10-90, 10-80, 10-70, 10-60, 10-50, 10-45, 10-40, 10-35, 10-30, 15-30, 15-35, 15-40, 15-45, 15-50, 20-35, 20-40, 20-45, or 20-50 nucleotides long. For example, the probe binding sequence of the DNA template may be at least or about 10, 15, 20, 25, 30, 35, 40, 45, 50, 55, 60, 65, 70, 75, 80, 85, 90, 95, 100, 120, 140, 160, 180, 200, 220, 240, 260, 280, or 300 nucleotides long.

In some aspects, the DNA polymerase is a high-fidelity DNA polymerase with 3′ to 5′ exonuclease activity.

In some aspects, the composition may comprise a population of non-extensible oligonucleotides and a population of DNA templates, wherein various non-extensible oligonucleotides of the population have different binding sequences that are at least 70% identical to the reverse complements of various probe binding sequences found within the population of DNA templates. For example, the composition may comprise at least or about 2, 3, 4, 5, 6, 7, 8, 9, 10, 11, 12, 13, 14, 15, or 20 different non-extensible oligonucleotides.

In one embodiment, provided herein are methods for selectively inhibiting a polymerase chain reaction (PCR) amplification of a template DNA having a selected sequence, the method comprising: (a) mixing a composition of any one of the present embodiments, a forward primer, a reverse primer, and dNTPs under conditions suitable for DNA polymerase activity; and (b) subjecting the mixture to at least 7 rounds of thermal cycling. For example, the thermal cycling may be performed for at least or about 7, 8, 9, 10, 11, 12, 13, 14, 15, 16, 17, 18, 19, 20, 25, 30, 35, or 40 cycles.

In some aspects, each round of thermal cycling comprises first holding the mixture at a temperature of at least 78 C for between 1 second and 30 minutes and then holding the mixing at a temperature of at most 75 C for between 1 second and 4 hours. For example, the first step may comprise holding the mixture at a temperature of at least 78 C, 79 C, 80 C, 81 C, 82 C, 83 C, 84 C, 85 C, 86 C, 87 C, 88 C, 89 C, 90 C, 91 C, 92 C, 93 C, 94 C, 95 C, 96 C, 97 C, or 98 C. For example, the first step may comprise holding at the temperature for between 1 second-30 minutes, 10 seconds-30 minutes, 20 seconds-30 minutes, 30 seconds-30 minutes, 45 seconds-30 minutes, 1 minute-30 minutes, 2-minutes-30 minutes, 30 second-5 minutes, or 1 minute-5 minutes. For example the first step may comprise holding at the temperature for at least or about 1 second, 5 seconds, 10 seconds, 15 seconds, 20 seconds, 30 seconds, 45 seconds, 1 minutes, 2 minutes, 5 minutes, or 10 minutes. For example, the second step may comprise holding the mixture at a temperature of at most 75 C, 74 C, 73 C, 72 C, 71 C, 70 C, 69 C, 68 C, 67 C, 66 C, 65 C, 64 C, 63 C, 62 C, 61 C, 60 C, 59 C, 58 C, 57 C, 56 C, or 55 C. For example, the second step may comprise holding at the temperature for between 1 second-4 hours, 1 second-3 hours, 1 second-2 hours, 1 second-1 hour, 1 second-30 minutes, 10 seconds-30 minutes, 20 seconds-30 minutes, 30 seconds-30 minutes, 45 seconds-30 minutes, 1 minute-30 minutes, 2-minutes-30 minutes, 30 second-5 minutes, or 1 minute-5 minutes. For example the first step may comprise holding at the temperature for at least or about 1 second, 5 seconds, 10 seconds, 15 seconds, 20 seconds, 30 seconds, 45 seconds, 1 minutes, 2 minutes, 5 minutes, or 10 minutes.

In some aspects, the forward primer is between 12 and 60 nucleotides long. In some aspects, the forward primer is at least 80%, 85%, 90%, 95%, 96%, 97%, 98%, or 99% identical to the reverse complement of a subsequence of the DNA template. In some aspects, the reverse primer is between 12 and 60 nucleotides long. In some aspects, the reverse primer is at least 80%, 85%, 90%, 95%, 96%, 97%, 98%, or 99% identical to a subsequence of the DNA template.

In some aspects, the DNA template optionally comprises a target DNA template. In some aspects, the DNA template comprises a background DNA template.

In some aspects, the non-extensible oligonucleotide has a binding sequence that is at least 80%, 85%, 90%, 95%, 96%, 97%, 98%, or 99% homologous to the reverse complement of the probe binding sequence of the background DNA template. In some aspects, the non-extensible oligonucleotide does not comprise an artificial chemical modification or a non-natural DNA nucleotide at its 3′ end.

In some aspects, the background DNA template is a pseudogene. In some aspects, the target DNA template is a gene sequence with above 80%, 85%, 90%, 95%, 96%, 97%, 98%, or 99% homology to the pseudogene. In some aspects, the background DNA template is a wildtype gene sequence. In some aspects, the target DNA template is a variant gene sequence with a single nucleotide replacement, a two-nucleotide replacement, an insertion of between 1 and 50 nucleotides, or a deletion of between 1 and 50 nucleotides. For example, an insertion or deletion may be 1, 2, 3, 4, 5, 6, 7, 8, 9, 10, 11, 12, 13, 14, 15, 20, 25, 30, 35, 40, 45, or 50 nucleotides long.

In some aspects, step (a) is performed using the composition of any one of the T1NEOs of the present embodiments. In some aspects, step (a) is performed using the composition of any one of the T2NEOs of the present embodiments.

In some aspects, the binding sequence of the non-extensible oligonucleotide is at least 70%, 75%, 80%, 85%, 90%, 95%, 96%, 97%, 98%, or 99% homologous to a 15 nucleotide subsequence of the forward primer. In some aspects, the mixture of step (a) comprises between 100 pM and 5 µM of the forward primer, between 100 pM and 5 µM of the reverse primer, and between 100 pM and 5 µM of the non-extensible oligonucleotide. For example, the concentration of any of the forward primer, reverse primer, and non-extensible oligonucleotide in the mixture, independently, may be between 100 pM-5 µM, 200 pM-5 µM, 300 pM-5 µM, 400 pM-5 µM, 500 pM-5 µM, 750 pM-5 µM, 1 nM-5 µM, 250 nM-5 µM, 500 nM-5 µM, 750 nM-5 µM, 1 µM-5 µM, 100 pM-1 µM, 200 pM-1 µM, 300 pM-1 µM, 400 pM-1 µM, 500 pM-1 µM, 750 pM-1 µM, 1 nM-1 µM, or 500 pM-500 nM. For example, the concentration of any of the forward primer, reverse primer, and non-extensible oligonucleotide in the mixture, independently, may be at least or about 100 pM, 200 pM, 300 pM, 400 pM, 500 pM, 750 pM, 1 nM, 10 nM, 50 nM, 100 nM, 200 nM, 300 nM, 400 nM, 500 nM, 750 nM, 1 µM, 2 µM, 3 µM, 4 µM, or 5 µM.

In some aspects, the DNA polymerase is a high-fidelity DNA polymerase with 3′ to 5′ exonuclease activity.

In some aspects, the mixture of step (a) further comprises an intercalating dye DNA or a Taqman probe. In some aspects, the quantity or concentration of the target DNA template is determined based on the cycle threshold (Ct) value.

In some aspects, the forward primer further comprises a forward adapter at its 5′ end, and the reverse primer further comprises a reverse adapter at its 5′ end, and the method further comprises (c) performing high-throughput sequencing. In some aspects, the method further comprises (c) ligating an adapter sequence to the PCR product produced in step (b), and (d) performing high-throughput sequencing.

In some aspects, the methods may be performed using a population of non-extensible oligonucleotides to amplify a population of DNA templates having various elected sequences, wherein various non-extensible oligonucleotides of the population have different binding sequences that are at least 70% identical to the reverse complements of various probe binding sequences found within the population of DNA templates. For example, the methods may be performed using at least or about 2, 3, 4, 5, 6, 7, 8, 9, 10, 11, 12, 13, 14, 15, or 20 different non-extensible oligonucleotides.

In some aspects, when methods require quantitative PCR, said can be performed using the reaction protocols and conditions provided in Example 1. In some aspects, when methods require next generation sequencing, said can be performed using the reaction protocols and conditions provided in Example 2. In some aspects, when methods require NGS bioinformatic analysis, said can be performed using the methods provided in Example 3. In some aspects, when methods require fold-enrichment analysis or variant allele frequency analysis, said can be performed using the methods provided in Example 4.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Key components of invention including Type 1 Non-extensible oligonucleotide (T1NEO). The dotted frame denotes the T1NEO. The DNA Template has, continuously from 5′ to 3′, an Upstream Sequence and a Probe Binding Sequence continuously. The gray arrow on the left side of Template Sequence and right side of the T1NEO denotes the 3′ end of the oligonucleotides. The Binding Sequence on the T1NEO and the Probe Binding Sequence on the Template are mostly or fully reverse complementary. The Terminator Hairpin at the T1NEO’s 3′-most region has a First Stem Sequence that is reverse complementary to the Second Stem Sequence. The Terminator Hairpin has a Loop Sequence between First Stem and Second Stem Sequences, illustrated as an arc on the top of Terminator Hairpin. The system also includes a DNA polymerase.

FIG. 2: Two embodiments of T1NEO. The top embodiment has Terminator Hairpin sequence 5′-TCTCGCAAGAGA-3′ (SEQ ID NO: 244). The bottom embodiment has Terminator Hairpin sequence 5′-GTTCGCAAGAAC-3′ (SEQ ID NO: 245).

FIG. 3: T1NEO with a Mismatch Sequence (MS) between the Binding Sequence and the Terminator Hairpin. The MS is not mostly or fully reverse complementary to the Upstream Sequence of the DNA Template.

FIG. 4: T1NEO with a MS comprising a hairpin. As shown, the MS comprises a former subsequence and a latter subsequence with reverse complementary sequence. The MS may additionally comprise additional subsequences that do not form a hairpin.

FIG. 5: T1NEO with a MS comprising stacked hairpins or multiple hairpins. In both embodiments, the T1NEO comprises a First Subsequence, Second Subsequence, Third Subsequence, and Fourth Subsequence, in order from 5′ to 3′. In the top embodiment, the First Subsequence is reverse complementary to the Fourth Subsequence, and the Second Subsequence is reverse complementary to the Third Subsequence, forming stacked hairpins. In the bottom embodiment, the first Subsequence is reverse complementary to the Second Subsequence, and the Third Subsequence is reverse complementary to the Fourth Subsequence, forming two independent hairpins.

FIG. 6: Experimental demonstration of T1NEO with a 10nt unstructured MS, and a Terminator Hairpin comprising sequence 5′-TCTCGCAAGAGA-3′ (SEQ ID NO: 244). Here, quantitative PCR (qPCR) was applied to a NA18537 human genomic DNA templates using the Phusion high-fidelity DNA polymerase with 3′->5′ exonuclease activity and using Syto-13 intercalating dye. In the gray lines, a forward primer (FP; 5′-GAGGGGTATTAGAAGAATGACTATGTGA-3′; SEQ ID NO: 85) and a reverse primer (RP; 5′-ACATGGTTAGATATTAGCCTGACCTATG-3′; SEQ ID NO: 165) are shown to effectively perform qPCR amplification. In contrast, when the FP is replaced by a T1NEO (5′-GACTATGTGACAAAATAGCTAAGGATACAGGAAATATGGAACATTAGTTCTCGC AAGAGA-3′; SEQ ID NO: 246), no observable PCR amplification occurs even when a 10-fold higher concentration of T1NEO is used. These data support the hypothesis that T1NEO is unlikely to be enzymatically extended even by DNA polymerases with 3′->5′ exonuclease activity. The late fluorescence increases in one of the three T1NEO traces may be due to RP primer dimer or nonspecific amplification on the genome.

FIG. 7: Experimental demonstration of additional embodiments of T1NEOs. The top embodiment shows a T1NEO with no MS (5′-GACTATGTGACAAAATAGCTAAGGATACAGGAAATATGTCTCGCAAGAGA-3′; SEQ ID NO: 246). The middle embodiment shows a T1NEO with a different Terminator Hairpin sequence (5′-GACTATGTGACAAAATAGCTAAGGATACAGGAAATATGGAACATTAGTACTCGC AAGAGT-3′; SEQ ID NO: 247). The bottom embodiment shows a T1NEO with a third Terminator Hairpin sequence (5′-GACTATGTGACAAAATAGCTAAGGATACAGGAAATATGGAACATTAGTGTTCGC AAGAAC-3′; SEQ ID NO: 248). All three embodiments are effective at suppressing DNA polymerase extension, but the bottom embodiment appears to be best for this genomic locus.

FIG. 8: Experimental demonstration of additional embodiments of T1NEOs comprising multiple hairpins and stacked hairpins in the MS. Both embodiments shown here (Top: 5′-GACTATGTGAcAAAATAGCTAAGGATACAGGAAATATGCGTAAGTCATCTTACGT GAGAGAACATTAGTTCTCCTTCTCCGTTGAGA-3′; SEQ ID NO: 249; Middle & Bottom: 5′-GACTATGTGAcAAAATAGCTAAGGATACAGGAAATATGGAGAACATTACCTGTA TGATACAGGCACAGTTCTCCTTCTCCGTTGAGA-3′; SEQ ID NO: 250) exhibit near-perfect suppression of DNA polymerase extension for this genomic locus. The stacked hairpin T1NEO was tested with both 100 nM and 1000 nM T1NEO; both experiments showed near-perfect suppression of Phusion DNA polymerase extension, demonstrating that the lack of qPCR signal is not due to nonspecific inhibition from high concentrations of T1NEO. See also FIGS. 16-18 for additional experimental evidence that T1NEO does not nonspecifically inhibit qPCR.

FIG. 9: Illustration of difficulty of constructing oligonucleotides that are not extensible by DNA polymerases with 3′->5′ exonuclease activity. Typically, any nucleotides at the 3′ end of a DNA primer that are mismatched to the Template Upstream Region will be removed by the DNA polymerase, and the matched DNA nucleotides on the primer will be extended. The Terminator Hairpin of the T1NEO prevents the DNA polymerase from recognizing and processively cleaving the 3′ nucleotides of the T1NEO.

FIG. 10: Oligonucleotides that less effectively or ineffectively suppress enzymatic extension by DNA polymerases with 3′->5′ exonuclease activity. The first and second top left diagrams illustrate primers with a 3-carbon spacer (5′-TAAACACCAAGACGTGGTAAATATTTACCTGG/3SpC3/; SEQ ID NO: 251) or a 4nt-TA tail sequence (5′-GACTATGTGACAAAATAGCTAAGGATACAGGAAATATTTAA-3′; SEQ ID NO: 252) at the 3′ end. Although these primer designs effectively prevent extension by Taq-based DNA polymerases, it is incapable of preventing extension by the Phusion DNA polymerase with 3′->5′ exonuclease activity. The middle and bottom diagram illustrate T1NEO designs (Middle: 5′-ACTGCTGcAGGCGCCCTGTACACTTTAACTCCGCAAGGA-3′; SEQ ID NO: 253; Bottom: 5′-ACTGCTGcAGGCGCCCTGTACACTTTAACTCAATCGCAAGATTGA-3′; SEQ ID NO: 254) with longer and shorter Terminator Hairpin stem sequences, or different loop sequence, that are less effective that preventing Phusion DNA polymerase extension.

FIG. 11: Key reagent components including Type 2 Non-extensible oligonucleotide (T2NEO). The dotted frame denotes the T2NEO. The DNA Template has, continuously from 5′ to 3′, an Upstream Sequence and a Probe Binding Sequence continuously. The gray arrow on the left side of Template Sequence and right side of the T2NEO denotes the 3′ end of the oligonucleotides. From 5′ to 3′, the T2NEO comprises a Binding Sequence, a Mismatch Sequence (MS), and a Tail Sequence. The Binding Sequence and the Probe Binding Sequence on the Template are mostly or fully reverse complementary. The MS comprises a First Stem Sequence and a Second Stem Sequence, where are reverse complementary to each other. The Tail Sequence is not homologous to the reverse complement of Upstream Sequence. The system also includes a DNA polymerase.

FIG. 12: Embodiments of T2NEO that comprise stacked hairpins, multiple hairpins, or sequences not in hairpin structures.

FIG. 13: Experimental demonstration of T2NEO with two hairpins in the MS, and 9nt Tail Sequence. Here, quantitative PCR (qPCR) was applied to a NA18537 human genomic DNA templates using the Phusion high-fidelity DNA polymerase with 3′->5′ exonuclease activity and using Syto-13 intercalating dye. In the gray lines, a forward primer (FP; 5′-ACCAATGGGAGTCACTGCTG-3′; SEQ ID NO: 84) and a reverse primer (RP; 5′-TAAGTGGAAAGAACTGGGGTGTC-3′; SEQ ID NO: 164) are shown to effectively perform qPCR amplification. In contrast, when the FP is replaced by a T2NEO (5′-ACTGCTGCAGGCGCCCTGTCTGAGAACATTAGTTCTCAGCCTGAGAACATTAGTT CTCAGTACCCCACT-3′; SEQ ID NO: 255), no observable PCR amplification occurs even when a 10-fold higher concentration of T2NEO is used. This data supports the hypothesis that T2NEO is unlikely to be enzymatically extended even by DNA polymerases with 3′->5′ exonuclease activity. The slow and late fluorescence increase in the T2NEO traces may be due to RP primer dimer or nonspecific amplification on the genome.

FIG. 14: Embodiment and experimental demonstration of a T2NEO with a branched hairpin structure. (5′-GACTATGTGAcAAAATAGCTAAGGATACAGGAAATAT ACGCAGG CTGAGAACATTAGTTCTCAG TGACCCTAATTATAGGGTCA CCTGCGT TGGCAAGAG-3′; SEQ ID NO: 256).

FIG. 15: Use of NEOs as Blockers for variant allele enrichment by Blocker Displacement Amplification (BDA). In BDA, a non-extensible Blocker oligonucleotide overlaps in sequence with a Forward Primer, so that the Blocker and Forward Primer compete in binding to DNA templates. The Blocker is designed to preferentially binds to a Background DNA Template (wildtype), and binds to the Target DNA Template (variant) less favorably. Thus, the Forward Primer will preferentially amplify the Target DNA Template, allowing enrichment of amplicons from the Target DNA Template over amplicons from the Background DNA Template. If the Blocker is enzymatically extended, then a significant portion of the amplicons will correspond to the Blocker-extension products, reducing the effectiveness of BDA enrichment.

FIG. 16: Experimental demonstration of BDA using a T1NEO as a Blocker in BDA. Here, the NA18537 human genomic DNA serves as the Background DNA Template, and the NA18562 human genomic DNA serves as the Target DNA Template. The T1NEO (5′-ACTGCTGCAGGCGCCCTGT CGTAAGTCAT TGA GAGAACATTAGTTCTC CT TATA GCAA GAGA-3′; SEQ ID NO: 257) covers the rs10230708 single nucleotide polymorphism (SNP) locus, in which NA18537 is homozygous for the G allele on the Template Sequence corresponding to the C nucleotide on the T1NEO, and NA18562 is homozygous for the T allele which is mismatched against T1NEO. In the absence of T1NEO, both NA18537 and NA18562 amplify effectively with cycle threshold (Ct) values of about 23.3. When T1NEO is present, the NA18562 gDNA is still amplified effectively with a Ct of 24.3, but the NA18537 gDNA is suppressed from amplification, with a Ct value of 38.1.

FIG. 17: Experimental demonstration of BDA using a T2NEO as a Blocker in BDA. The T2NEO (5′-ACTGCTGCAGGCGCCCTGTCTGAGAACATTAGTTCTCAGCCTGAGAACATTAGTT CTCAGTACCCCACT-3′; SEQ ID NO: 255) covers the rs10230708 single nucleotide polymorphism (SNP) locus, in which NA18537 is homozygous for the G allele, and NA18562 is homozygous for the T allele.

FIG. 18: Embodiment of NEOs as a method for suppressing pseudogene amplification. The NEO is perfectly matched to pseudogene-specific sequences, and the Forward Primer is perfectly matched to corresponding true gene sequences.

FIG. 19: Embodiment of NEOs as hybrid-capture probes for NGS target enrichment. 5′-biotinylated NEO probes are bound to streptavidin-coated magnetic beads via biotin-streptavidin interaction, and used to selectively hybridize adapter-appended DNA molecules corresponding to genes of interest. In subsequent on-bead PCR amplification after hybrid-capture enrichment, the NEO probes are not extended.

FIG. 20: Key components of invention including a subtype of T1NEO and three embodiments of this subtype. In the top panel, the dotted frame denotes the structure of this subtype. The DNA Template has, continuously from 5′ to 3′, an Upstream Sequence and a Probe Binding Sequence continuously. The gray arrow on the left side of the Template Sequence and right side of the T1NEO subtype denotes the 3′ end of the oligonucleotides. The Biological Sequence on the subtype and the Probe Binding Sequence on the Template are mostly or fully reverse complementary. The Terminator Hairpin at the 3′-most region has a First Stem Sequence that is reverse complementary to the Second Stem Sequence. The Middle Hairpin between the Biological Sequence and the Terminator Hairpin has a Third Stem Sequence that is reverse complementary to the Fourth Stem Sequence. The Middle Hairpin and the Terminator Hairpin individually have a First Loop Sequence between the First and Second Stem Sequences, a Second Loop Sequence between the Third and Fourth Stem Sequences, illustrated as arcs on the top of the Terminator Hairpin and Middle Hairpin. The system also includes a DNA polymerase. From the second to the bottom panels, structures and sequences of three NEO sequences are shown. The second panel, MiddleA NEO Sequence, has sequence 5′-GAGAACATTAGTTCTC GTTAGCAATAAC-3′ (SEQ ID NO: 258). The third panel, MiddleB NEO Sequence, has sequence 5′-GAGAACATTAGTTCTC GATTGCAAAATC-3′ (SEQ ID NO: 259). The bottom panel, MiddleC NEO Sequence, has sequence 5′-CCTGTACACATACAGG GTTAGCAATAAC-3′ (SEQ ID NO: 260).

FIG. 21: Experimental demonstration of MiddleA, MiddleB, and MiddleC NEO Sequence. Here, quantitative PCR (qPCR) was applied to NA18537 human genomic DNA templates using the Phusion high-fidelity DNA polymerase with 3′->5′ exonuclease activity and using Syto-13 intercalating dye. In the gray lines, a forward primer (FP) and a reverse primer (RP) are shown to effectively perform qPCR amplification. In contrast, when the FP is replaced by any of MiddleA (SEQ ID NO: 81), MiddleB (SEQ ID NO: 2) or MiddleC (SEQ ID NO: 82) NEO Sequences, no observable PCR amplification occurs even when a 10-fold higher concentration of the NEO Sequence is used. These data support the hypothesis that none of these NEO Sequences are unlikely to be enzymatically extended even by DNA polymerases with 3′->5′ exonuclease activity.

FIG. 22: Use of NEO Sequence in BDA qPCR and BDA NGS. As shown in the top panel, the NA18537 human genomic DNA serves as the Background DNA Template, and the NA18562 human genomic DNA serves as the Target DNA Template. The MiddleC NEO Sequence (SEQ ID NO: 83) covers the rs10230708 single nucleotide polymorphism (SNP) locus, in which NA18537 is homozygous for the G allele on the Template Sequence corresponding to the C nucleotide on the MiddleC NEO Sequence, and NA18562 is homozygous for the T allele which is mismatched against MiddleC NEO Sequence. When MiddleC NEO Sequence is present, the NA18562 gDNA is amplified effectively with a Ct of 23.3, but the NA18537 gDNA is suppressed from amplification, with a Ct value of 33.4. As shown in the bottom panel, there is a summary of experimental NGS results using 80-plex PCR target enrichment. Here, 80 different forward primers (Table 2) and 80 different reverse primers (Table 3) were designed to 80 distinct regions of the human genome. Then 80 MiddleB NEO Sequence blockers (Table 1) with different Biological Sequence were designed to enrich variant amplicons. For the same 0.7% VAF sample, almost 200-fold more variant will be enriched by MiddleB NEO Sequence.

### DETAILED DESCRIPTION

Disclosed herein are non-extensible oligonucleotides (NEOs) with rationally designed secondary structures at or near their 3′ end. These NEOs do not have 3′ chemical modifications, and are not extended enzymatically by high fidelity DNA polymerases with 3′->5′ exonuclease activity. These NEOs can be combined with many current methods, achieving lower cost. For example, NEOs can be used with blocker displacement amplification (BDA) technology on qPCR, next-generation sequencing, and nanopore sequencing. As another example, NEOs can be used in hybrid-capture probe sets for NGS target enrichment.

### L Definitions

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

“Incorporating,” as used herein, means becoming part of a nucleic acid polymer.

The term “in the absence of exogenous manipulation” as used herein refers to there being modification of a nucleic acid molecule without changing the solution in which the nucleic acid molecule is being modified. In specific embodiments, it occurs in the absence of the hand of man or in the absence of a machine that changes solution conditions, which may also be referred to as buffer conditions. However, changes in temperature may occur during the modification.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” “Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.” In addition, the term “adaptor” can indicate a linear adaptor (either single stranded or double stranded) or a stem-loop adaptor. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

A “nucleic acid molecule” or “nucleic acid target molecule” refers to any single-stranded or double-stranded nucleic acid molecule including standard canonical bases, hypermodified bases, non-natural bases, or any combination of the bases thereof. For example and without limitation, the nucleic acid molecule contains the four canonical DNA bases - adenine, cytosine, guanine, and thymine, and/or the four canonical RNA bases -adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin. Examples of nucleic acid molecules include genomic DNA, cDNA, RNA, a DNA/RNA hybrid, amplified DNA, a pre-existing nucleic acid library, etc. A nucleic acid may be obtained from a human sample, such as blood, serum, plasma, cerebrospinal fluid, cheek scrapings, biopsy, semen, urine, feces, saliva, sweat, etc. A nucleic acid molecule may be subjected to various treatments, such as repair treatments and fragmenting treatments. Fragmenting treatments include mechanical, sonic, and hydrodynamic shearing. Repair treatments include nick repair via extension and/or ligation, polishing to create blunt ends, removal of damaged bases, such as deaminated, derivatized, abasic, or crosslinked nucleotides, etc. A nucleic acid molecule of interest may also be subjected to chemical modification (e.g., bisulfite conversion, methylation / demethylation), extension, amplification (e.g., PCR, isothermal, etc.), etc.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

The term “non-complementary” refers to nucleic acid sequence that lacks the ability to form at least one Watson-Crick base pair through specific hydrogen bonds.

The term “degenerate” as used herein refers to a nucleotide or series of nucleotides wherein the identity can be selected from a variety of choices of nucleotides, as opposed to a defined sequence. In specific embodiments, there can be a choice from two or more different nucleotides. In further specific embodiments, the selection of a nucleotide at one particular position comprises selection from only purines, only pyrimidines, or from non-pairing purines and pyrimidines.

The term “secondary structure” as used herein refers to the set of interactions between bases pairs. For example, in a DNA double helix, the two strands of DNA are held together by hydrogen bonds. The secondary structure is responsible for the shape that the nucleic acid assumes. For a single stranded nucleic acid, the simplest secondary structure is linear. For a linear secondary structure, no two subsequences of a nucleic acid molecule form an intramolecular structure stronger than -2 kcal/mol. As another example for a single stranded nucleic acid, one portion of the nucleic acid molecule may hybridize with a second portion of the same nucleic acid molecule, thereby forming a hairpin to stem loop secondary structure. For a non-linear secondary structure, at least two subsequences of a nucleic acid molecule from an intramolecular structure stronger than -2 kcal/mol.

As used herein, the term “blocker oligonucleotide” refers to at least one continuous strand of from about 12 to about 100 nucleotides in length and if so indicated herein, may further include a functional group or nucleotide sequence at its 3′ end that prevents enzymatic extension during an amplification process such as polymerase chain reaction.

As used herein, the term “primer oligonucleotide” refers to a molecule comprising at least one continuous strand of from about 12 to about 100 nucleotides in length and sufficient to permit enzymatic extension during an amplification process such as polymerase chain reaction.

As used herein, the term “target-neutral subsequence” refers to a sequence of nucleotides that is complementary to a sequence in both a target nucleic acid and a background nucleic acid. For example, a desired nucleic acid sequence to be targeted for amplification (target nucleic acid) may exist in a sample with a nucleic acid molecule having a predominantly homologous sequence with the target nucleic acid with the exception of a variable region (background nucleic acid), such variable region in some instance being only a single nucleotide difference from the target nucleic acid. In this example, the target-neutral subsequence is complementary to at least a portion of the homologous sequence shared between the two nucleic acids, but not the variable region. Thus, as used herein, the term “blocker variable subsequence” refers to a nucleotide sequence of a blocker oligonucleotide which is complementary to the variable region of the background nucleic.

As used herein, the term “overlapping subsequence” refers to a nucleotide sequence of at least 5 nucleotides of a primer oligonucleotide that is homologous with a portion of the blocker oligonucleotide sequence used in a composition as described herein. The overlapping subsequence of the primer oligonucleotide may be homologous to any portion of the target-neutral subsequence of the blocker oligonucleotide, whether 5′ or 3′ of the blocker variable subsequence. Thus, the term “non-overlapping subsequence” refers to the sequence of a primer oligonucleotide that is not the overlapping subsequence.

As used herein, the term “target sequence” refers to the nucleotide sequence of a nucleic acid that harbors a desired allele, such as a single nucleotide polymorphism, to be amplified, identified, or otherwise isolated. As used herein, the term “background sequence” refers to the nucleotide sequence of a nucleic acid that does not harbor the desired allele. For example, in some instances, the background sequence harbors the wild-type allele whereas the target sequence harbors the mutant allele. Thus, in some instance, the background sequence and the target sequence are derived from a common locus in a genome such that the sequences of each may be substantially homologous except for a region harboring the desired allele, nucleotide or group or nucleotides that varies between the two. In another example, in some instances, the background sequence harbors a pseudogene sequence whereas the target sequence harbors the true gene sequence.

“Sample” means a material obtained or isolated from a fresh or preserved biological sample or synthetically created source that contains nucleic acids of interest. Samples can include at least one cell, fetal cell, cell culture, tissue specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals, other animals, plants, fungi, bacteria, and viruses.

As used herein in relation to a nucleotide sequence, “substantially known” refers to having sufficient sequence information in order to permit preparation of a nucleic acid molecule, including its amplification. This will typically be about 100%, although in some embodiments some portion of an adaptor sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

### II. Non-Extensible Oligonucleotide (NEOs)

### A. Type 1 NEOs

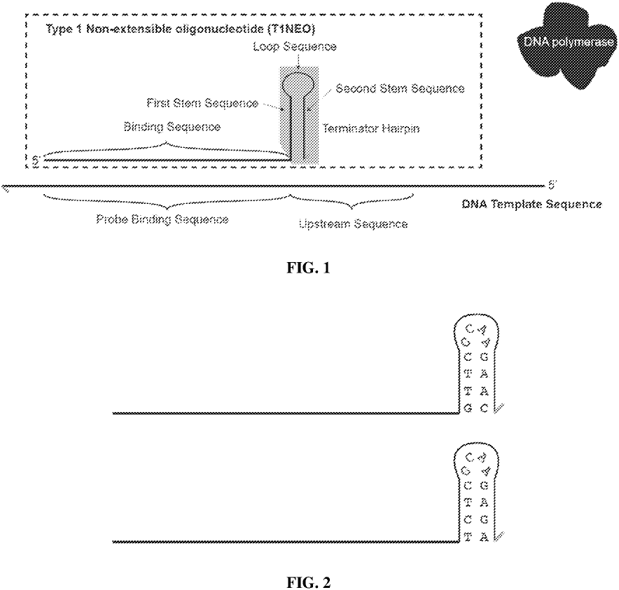

A Type 1 Non-Extensible Oligonucleotide (T1NEO) has a 5′ Binding Sequence that exhibits significant sequence similarity to the reverse complement of a Probe Binding Sequence of DNA Template Sequence (FIG. 1). The T1NEO Binding Sequence may be 70%, 75%, 80%, 85%, 90%, 91%, 92%, 93%, 94%, 95%, 96%, 97%, 98%, 99%, or 100% identical to the reverse complement of a Probe Binding Sequence of a DNA Template Sequence. The 3′-most region of a T1NEO has a Terminator Hairpin region comprising a Loop Sequence positioned between a First Stem Sequence and a Second Stem Sequence. The First Stem Sequence and the Second Stem Sequence are reverse complementary to each other. The Terminator Hairpin is not reverse complementary to Upstream Sequence of the DNA Template Sequence. In one embodiment, the Terminator Hairpin has a sequence 5′-TCTC GCAA GAGA-3′ (SEQ ID NO: 244; FIG. 2).

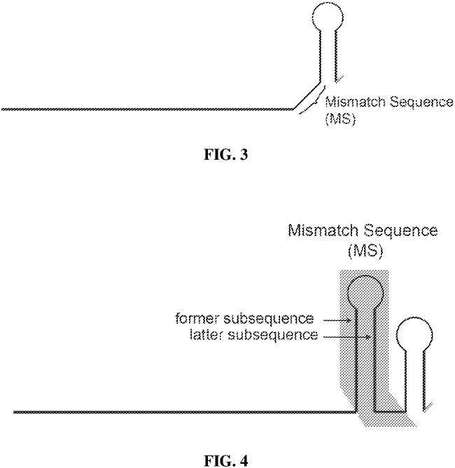

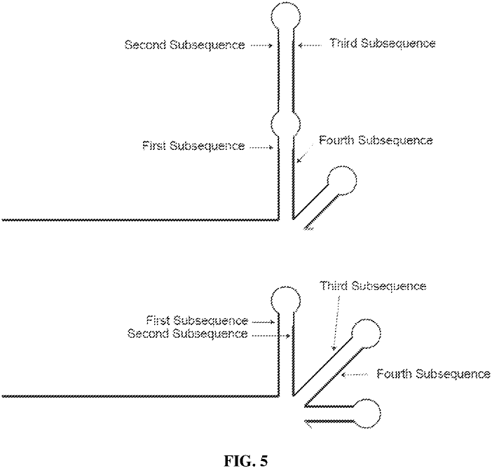

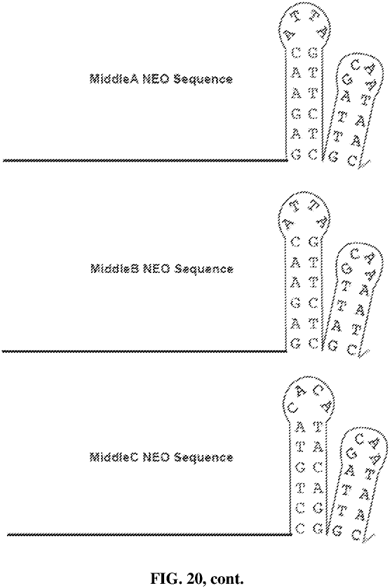

Variant embodiments of the T1NEO are shown in FIGS. 3-5. In these variant embodiments, the T1NEO further comprises a Mismatch Sequence (MS) positioned between the Binding Sequence and the Terminator Hairpin. This MS may comprise two or more hairpin structures (FIG. 5), one hairpin structure (FIG. 4), or no hairpin structures (FIG. 3). In addition, FIG. 20 shows a specific subtype of T1NEO that comprises a Middle Hairpin between the Biological (Binding) Sequence and the Terminator Hairpin.

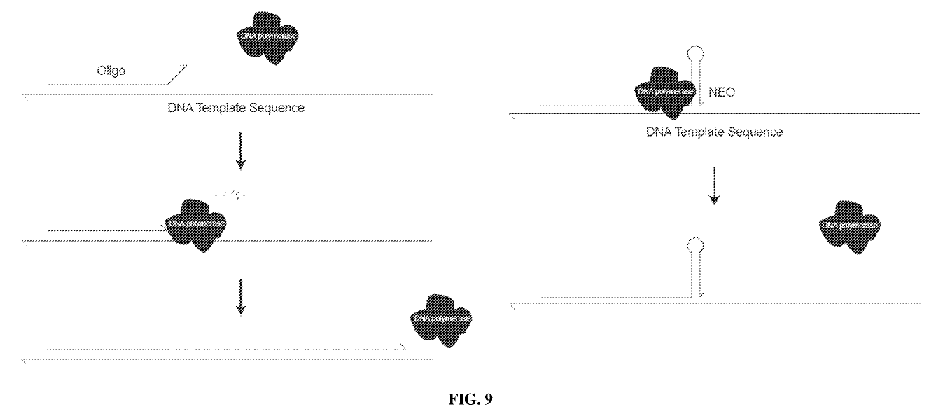

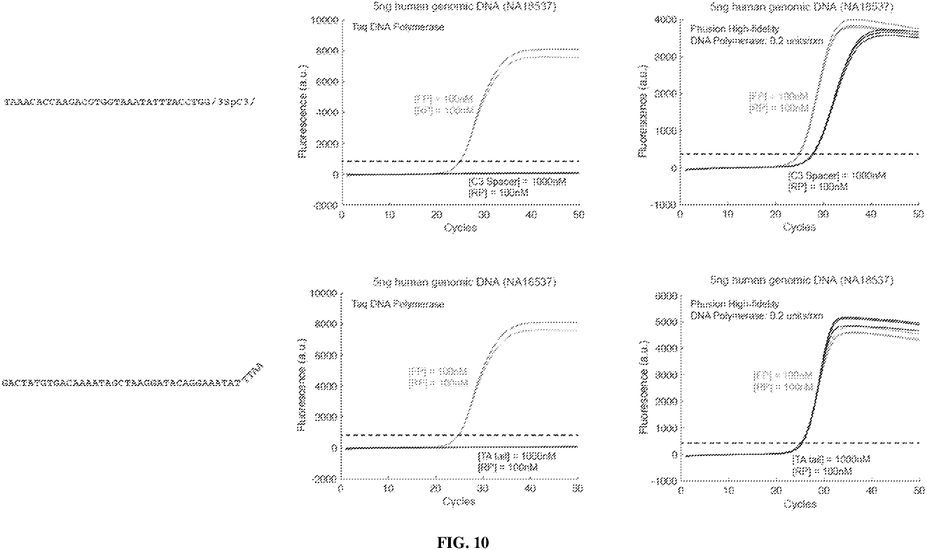

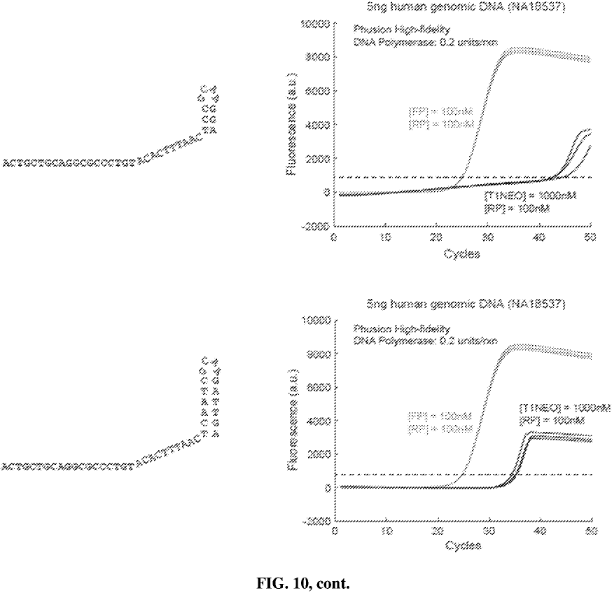

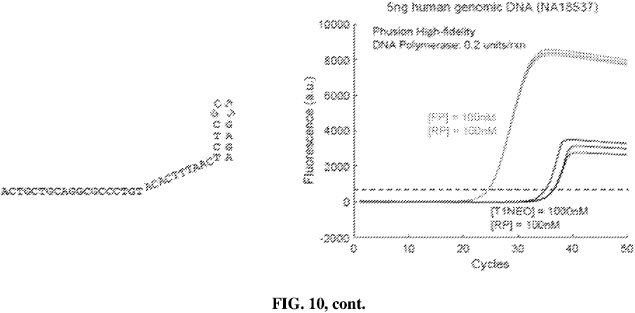

The 3′->5′ exonuclease activity of high-fidelity DNA polymerases is a critical feature that enables these enzymes to be used for detection and quantitation of mutations with low variant allele frequencies (VAFs), such as somatic mutations in tumor tissue or cell-free DNA. The 3′->5′ exonuclease activity allows kinetic proofreading, whereby incorrectly incorporated DNA nucleotides at the 3′ end of a growing amplicon can be removed, and enables DNA polymerases such as Phusion and Q5 to exhibit misincorporation error rates that are between 20- and 200-fold lower than Taq-based DNA polymerases. However, this 3′->5′ exonuclease activity also renders it challenging to design DNA probes and blockers that are not intended to be enzymatically extended (FIG. 9). Even many 3′ chemical modifications that prevent Taq extension are not effective at preventing extension after 3′->5′ exonuclease activity. FIG. 10 shows a series of DNA oligos with and without 3′ chemical modifications that are less effective at preventing enzymatic extension by DNA polymerases with 3′->5′ exonuclease activity.

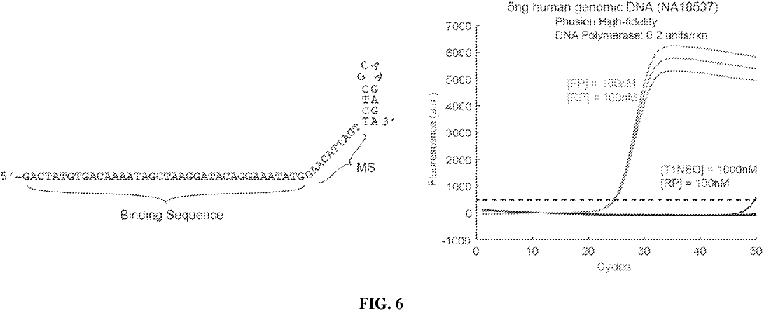

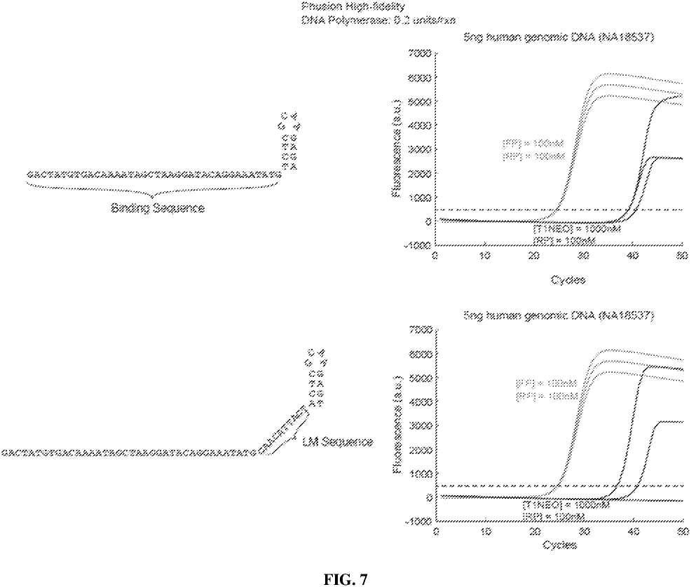

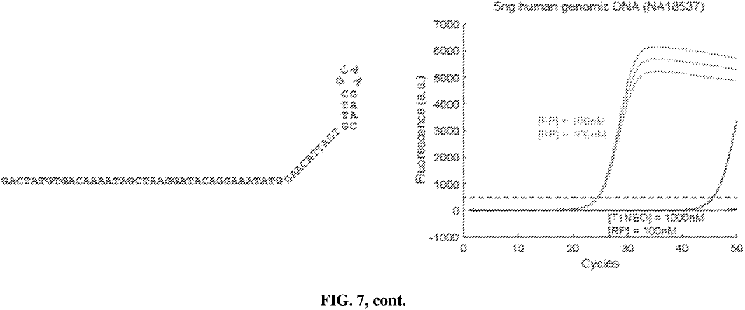

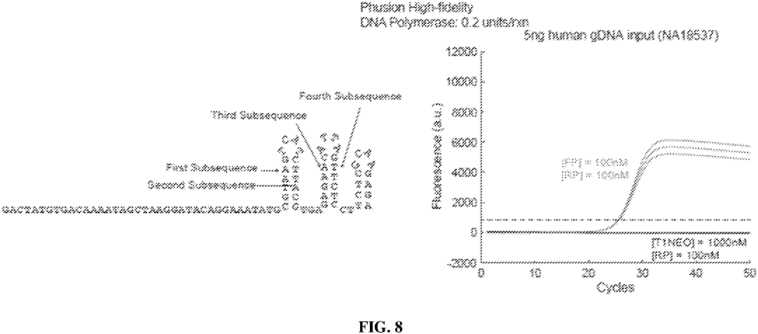

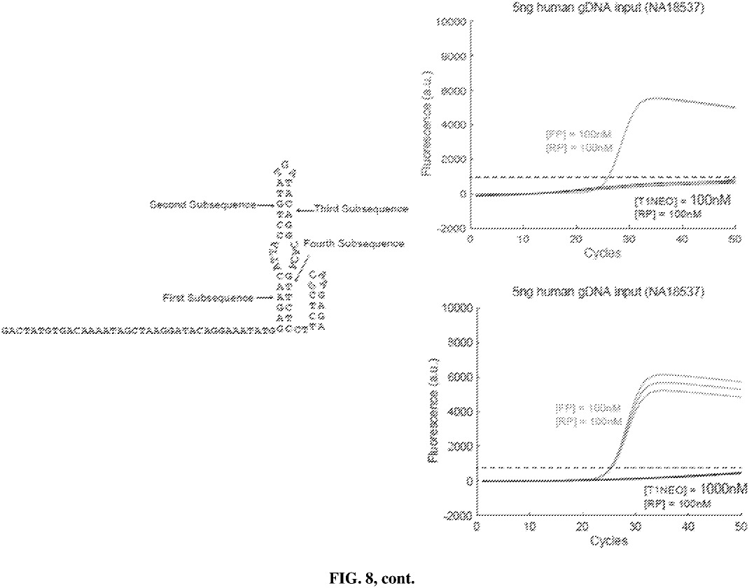

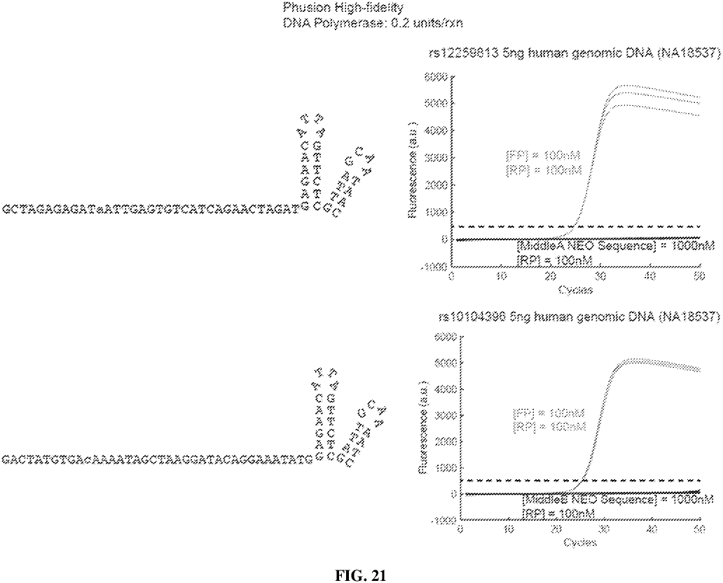

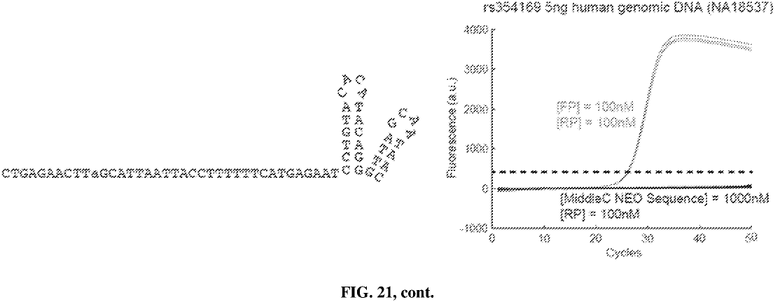

However, T1NEOs cannot be effectively extended by DNA polymerases, including by high fidelity DNA polymerases with 3′->5′ exonuclease activity. A number of quantitative PCR (qPCR) experiments were performed using a T1NEO and a Reverse Primer (ACATGGTTAGATATTAGCCTGACCTATG; SEQ ID NO: 165) in order to demonstrate this (FIGS. 6-8). No qPCR amplification or very late amplification indicated that the T1NEO was not enzymatically extended. In contrast, using a Forward Primer (GAGGGGTATTAGAAGAATGACTATGTGA; SEQ ID NO: 85) with similar sequence to the T1NEO, but lacking the Terminator Hairpin, showed effective qPCR amplification and detection, indicating that the primer designs, DNA polymerases, and DNA input sample are all compatible with PCR amplification. In addition, a number of quantitative PCR (qPCR) experiments were performed using three listed sequences (MiddleA, MiddleB, MiddleC NEO Sequence) and their corresponding Reverse Primers (FIG. 21). For the T1NEO that comprises a Middle Hairpin, no qPCR amplification or very late amplification indicates that this subtype is not enzymatically extended. In contrast, using a Forward Primer with similar sequence to the NEO Sequences, but lacking the Middle Hairpin and Terminator Hairpin, shows effective qPCR amplification and detection, indicating that the primer designs, DNA polymerases, and DNA input sample are all compatible with PCR amplification. In some embodiments, intercalating DNA dyes (e.g., Syto-13) that produce fluorescence nonspecifically to buildup of dsDNA amplicons can be used to detect amplification. In other embodiments, the qPCR reactions comprise a Taqman probe rather than intercalating dyes to report on specific amplicon buildup.

TABLE 1

Exemplary T1NEO with middle hairpins

| T1NEO | Sequence | SEQ ID NO |
|---|---|---|
| MiddleB NEO_rs10230708 | ACTGCTGcAGGCGCCCTGT GAGAACATTAGTTCTC GATTGCAAAATC | 1 |
| MiddleB NEO_rs10104396 | GACTATGTGAcAAAATAGCTAAGGATACAG GAAATATG GAGAACATTAGTTCTC GATTGCAAAATC | 2 |
| MiddleB NEO_rs199032 | CATCTTTATTTAACCCaTTAGAAAATCCTA TCAGCTCT GAGAACATTAGTTCTC GATTGCAAAATC | 3 |
| MiddleB NEO_rs926850 | CCGTCATAACAAaAACATATTTACTTTCTC TGGC GAGAACATTAGTTCTC GATTGCAAAATC | 4 |
| MiddleB NEO_rs17149369 | CTTCAATATTGCaGAAGTGTTGCAAGCCT GAGAACATTAGTTCTC GATTGCAAAATC | 5 |
| MiddleB NEO_rs869720 | AGGGAGAGAACCTCCTcCCTCACAGA GAGAACATTAGTTCTC GATTGCAAAATC | 6 |
| MiddleB NEO_rs12478327 | TCAAATTCAGGTAcCTTAGAGGGACAGCTA AA GAGAACATTAGTTCTC GATTGCAAAATC | 7 |
| MiddleB NEO_rs2638145 | AATGCAAAACTcAATGTATCAGTGTGAGGA TGT GAGAACATTAGTTCTC GATTGCAAAATC | 8 |
| MiddleB NEO_rs2170091 | TAGCTTCAGAaACATTCCAGTGTATGTGCA G GAGAACATTAGTTCTC GATTGCAAAATC | 9 |
| MiddleB NEO_rs2043583 | GTTAGAGCAACTTTCCTTGATTCCCAGAGT AG GAGAACATTAGTTCTC GATTGCAAAATC | 10 |
| MiddleB NEO_rs955456 | CCTTGAAAAGAGGGCTTAGGTtTTCTTTGC GAGAACATTAGTTCTC GATTGCAAAATC | 11 |
| MiddleB NEO_rs966516 | CTTATGAAGTCATGGAACaATGCCTACTTC TATATTT GAGAACATTAGTTCTC GATTGCAAAATC | 12 |
| MiddleB NEO_rs354169 | CTGAGAACTTaGCATTAATTACCTTTTTTC ATGAGAAT GAGAACATTAGTTCTC GATTGCAAAATC | 13 |
| MiddleB NEO_rs1898170 | AGGGCAtTTTTTACAGTGTTGAATATTGAA ACTG GAGAACATTAGTTCTC GATTGCAAAATC | 14 |
| MiddleB NEO_rs11247921 | CTCTCATGGTATGgTGTTTTTCTGTGCTCC GAGAACATTAGTTCTC GATTGCAAAATC | 15 |
| MiddleB NEO_rs1635718 | CAGATGAAAATTATCTGTGCTTTTTTgTAA GCTGATATATT GAGAACATTAGTTCTC GATTGCAAAATC | 16 |
| MiddleB NEO_rs10510620 | CAATCTCTGAATCTcAGAATAGTAGCCTAG AAAACG GAGAACATTAGTTCTC GATTGCAAAATC | 17 |
| MiddleB NEO_rs7104025 | CTCATGAGTTAAcAAGGAGATGATGTAGTG TAAAG GAGAACATTAGTTCTC GATTGCAAAATC | 18 |
| MiddleB NEO_rs2246745 | CAACAAACATGCCtTCTCCTTCTCCTGA GAGAACATTAGTTCTC GATTGCAAAATC | 19 |
| MiddleB NEO_rs3789806 | TAAACACCAAGAcGTGGTAAATATTTACCTGGT GAGAACATTAGTTCTC GATTGCAAAATC | 20 |
| MiddleB NEO_rs706714 | CAACAAGGTCAGTATTGATAaGTGGTTGCT GAGAACATTAGTTCTC GATTGCAAAATC | 21 |
| MiddleB NEO_rs1884444 | ACATGAATCAtGTCACTATTCAATGGGATG C GAGAACATTAGTTCTC GATTGCAAAATC | 22 |
| MiddleB NEO_rs2510152 | TTTTGTTTCACATgATAACCATATCACTGG ACACA GAGAACATTAGTTCTC GATTGCAAAATC | 23 |
| MiddleB NEO_rs16754 | AGGATGTGCGaCGTGTGCCTG GAGAACATTAGTTCTC GATTGCAAAATC | 24 |
| MiddleB NEO_rs206781 | GGTCCAAAGCCgGAAGGGCCTAAA GAGAACATTAGTTCTC GATTGCAAAATC | 25 |
| MiddleB NEO_rs28932178 | GCCTGGAACCGAGACGcCTCAG GAGAACATTAGTTCTC GATTGCAAAATC | 26 |
| MiddleB NEO_rs10186821 | TCCATTGGCTAcTCAGTCTCGGCT GAGAACATTAGTTCTC GATTGCAAAATC | 27 |
| MiddleB NEO_rs10508599 | TCATATTGAGCtTAAGAGTTCAGAACACTG ATGG GAGAACATTAGTTCTC GATTGCAAAATC | 28 |
| MiddleB NEO_rs10738578 | CATAATTGCATATAACCTAcACACATTCTC CCA GAGAACATTAGTTCTC GATTGCAAAATC | 29 |
| MiddleB NEO_rs10741037 | GTTATGTGCTGGAAAGAGcATAAATTTTGG AAT GAGAACATTAGTTCTC GATTGCAAAATC | 30 |
| MiddleB NEO_rs10770674 | CTCCTACTGTACATAcATATTATCTTAAGG AAAAAATCCAAAT GAGAACATTAGTTCTC GATTGCAAAATC | 31 |
| MiddleB NEO_rs10805227 | TGTTCAATGTATTAAATAATCaTCAGCATA TTTTTGTATTCAC GAGAACATTAGTTCTC GATTGCAAAATC | 32 |
| MiddleB NEO_rs10833604 | GATTGGTAGAAGAcACTGATTGCATCTTCA A GAGAACATTAGTTCTC GATTGCAAAATC | 33 |
| MiddleB NEO_rs10964389 | AAGGCACAGAACAATcATGCAACTTGC GAGAACATTAGTTCTC GATTGCAAAATC | 34 |
| MiddleB NEO_rs11015816 | GGGACTTTcTTGAGGGATGGCATCC GAGAACATTAGTTCTC GATTGCAAAATC | 35 |
| MiddleB NEO_rs11045749 | GAGGTGATATCTCaTTTTGGCTTCTATTTG CA GAGAACATTAGTTCTC GATTGCAAAATC | 36 |
| MiddleB NEO_rs1123828 | TGTCAAACACCCaTGCTCACCCTT GAGAACATTAGTTCTC GATTGCAAAATC | 37 |
| MiddleB NEO_rs11708584 | GGTCCTCTTTAAGGTCTCTaCAATAAATTG CCA GAGAACATTAGTTCTC GATTGCAAAATC | 38 |
| MiddleB NEO_rs12192635 | GACATAATGCTTTTGGTTGGACTTTCAaAA AGG GAGAACATTAGTTCTC GATTGCAAAATC | 39 |
| MiddleB NEO_rs12213948 | GCAAGGTTCAAATCATTCTCTCcTATCTCA TC GAGAACATTAGTTCTC GATTGCAAAATC | 40 |
| MiddleB NEO_rs12259813 | GCTAGAGAGATaATTGAGTGTCATCAGAAC TAGAT GAGAACATTAGTTCTC GATTGCAAAATC | 41 |
| MiddleB NEO_rs12541300 | ATGAGGAGTAATTGAAATCATTAATAcCCA CAAACA GAGAACATTAGTTCTC GATTGCAAAATC | 42 |
| MiddleB NEO_rs12681931 | AACTCAGACCaATTTGGCCATAGATTATTA GC GAGAACATTAGTTCTC | 43 |
| | GATTGCAAAATC | |
| MiddleB NEO_rs12782580 | ACAAAACCCTATAAGGAAGATGTCaTTACC CATATTTTA GAGAACATTAGTTCTC GATTGCAAAATC | 44 |
| MiddleB NEO_rs1375977 | ACCCAGCTTTATACaTTCACAAAGATATGG TTTG GAGAACATTAGTTCTC GATTGCAAAATC | 45 |
| MiddleB NEO_rs1516755 | ACAGTGGAACAGCTcTCTCCTTCTTTTTT GAGAACATTAGTTCTC GATTGCAAAATC | 46 |
| MiddleB NEO_rs1524303 | ATTAGAATAACTACTATTaAAAAAACCCCA CAAAATAACTCTT GAGAACATTAGTTCTC GATTGCAAAATC | 47 |
| MiddleB NEO_rs1667087 | TTTGGGAATTAAAAGCCAATAGATTAGCTG aAAATTC GAGAACATTAGTTCTC GATTGCAAAATC | 48 |
| MiddleB NEO_rs16871316 | ACACCTTTACATGaAGGCTTTGAAGTACTC TT GAGAACATTAGTTCTC GATTGCAAAATC | 49 |
| MiddleB NEO_rs16925478 | GTGCATTATGgGTAAGAATGTTCATTTATT ATTTCACTTATA GAGAACATTAGTTCTC GATTGCAAAATC | 50 |
| MiddleB NEO_rs17560702 | GAAGTCGTAGCTATTcGGCAAAGGAAATG GAGAACATTAGTTCTC GATTGCAAAATC | 51 |
| MiddleB NEO_rs1937037 | TGCCCCATAGGCAGTGTTTGgTGAAG GAGAACATTAGTTCTC GATTGCAAAATC | 52 |
| MiddleB NEO_rs2215492 | TACCCCATGTGTATcAAATGGTCAGCAAG GAGAACATTAGTTCTC GATTGCAAAATC | 53 |
| MiddleB NEO_rs2301720 | CTGTGAGTTGGGaGCAAAGGAGCA GAGAACATTAGTTCTC GATTGCAAAATC | 54 |
| MiddleB NEO_rs2616187 | CTCTGGAGAcGGGGGATGTTAAGTTGA GAGAACATTAGTTCTC GATTGCAAAATC | 55 |
| MiddleB NEO_rs2710998 | TCTGGTGATTGAGAAAGcGTTCCAGA GAGAACATTAGTTCTC GATTGCAAAATC | 56 |
| MiddleB NEO_rs2807238 | ATTGGATTAACTTTGGTGGAACcTACTTCG AT GAGAACATTAGTTCTC GATTGCAAAATC | 57 |
| MiddleB NEO_rs2874755 | CTCCCTTCTTTCATCCCTaCATCATGTCC GAGAACATTAGTTCTC GATTGCAAAATC | 58 |
| MiddleB NEO_rs3813787 | CGGACTTGGCTGGGGTaGAGCTT GAGAACATTAGTTCTC GATTGCAAAATC | 59 |
| MiddleB NEO_rs4665582 | GAGCTAAGTACCAGGTATGAtGCTCGC GAGAACATTAGTTCTC GATTGCAAAATC | 60 |
| MiddleB NEO_rs4712476 | AGGGAATGCTCTAgACAAAACACTGTTCC GAGAACATTAGTTCTC GATTGCAAAATC | 61 |
| MiddleB NEO_rs611628 | TGCTTTGTGCTaGCTCAAAGACTCACAT GAGAACATTAGTTCTC GATTGCAAAATC | 62 |
| MiddleB NEO_rs6452035 | AATTCTGGATCAAATTAAATATGTcGCATT CTCC GAGAACATTAGTTCTC GATTGCAAAATC | 63 |
| MiddleB NEO_rs6816854 | TGTACTTTCTTTTTAGCcATAAGATGATTT | 64 |
| | CCCAT GAGAACATTAGTTCTC GATTGCAAAATC | |
| MiddleB NEO_rs6937778 | GCTTGCTTTCCcACACCACTACCT GAGAACATTAGTTCTC GATTGCAAAATC | 65 |
| MiddleB NEO_rs7003044 | GGTCAAGTCTGAGGCTGTTGaGCTTA GAGAACATTAGTTCTC GATTGCAAAATC | 66 |
| MiddleB NEO_rs7032336 | TTCAGGACGTGAAAGCACGaGAACG GAGAACATTAGTTCTC GATTGCAAAATC | 67 |
| MiddleB NEO_rs7816009 | ATGTACAATTTCAAcTGGAGTTTCCATTGC A GAGAACATTAGTTCTC GATTGCAAAATC | 68 |
| MiddleB NEO_rs7893462 | AAATAGTGAGAAcGAGCAGCTGCAGG GAGAACATTAGTTCTC GATTGCAAAATC | 69 |
| MiddleB NEO₋rs7902135 | AAGAATATAAAATGTTAGAGAACCACATAc AACGAGC GAGAACATTAGTTCTC GATTGCAAAATC | 70 |
| MiddleB NEO_rs898476 | AACCCCAGAACaCTAGCAGCTAAGGG GAGAACATTAGTTCTC GATTGCAAAATC | 71 |
| MiddleB NEO_rs9368431 | TTTTATTAGTTGTGTAATCCAGTTACTTAa CTTTAAAAGCC GAGAACATTAGTTCTC GATTGCAAAATC | 72 |
| MiddleB NEO_rs9438621 | GTTCTGAAAAGAGcCTCCACTCCTGT GAGAACATTAGTTCTC GATTGCAAAATC | 73 |
| MiddleB NEO_rs9466035 | CCTCCACTCCACCaTGGCACCTATTA GAGAACATTAGTTCTC GATTGCAAAATC | 74 |
| MiddleB NEO_rs9466930 | GTATACCACTTAGGCTATAGTTATTcTAAA CTTTGATAAAC GAGAACATTAGTTCTC GATTGCAAAATC | 75 |
| MiddleB NEO_rs9973865 | AGGAATCATTACAGGaAAACATCGTTTAAA TTGGA GAGAACATTAGTTCTC GATTGCAAAATC | 76 |
| MiddleB NEO_rs4712498 | CCATGGTATATTGTAaGTTGTAGGTACATA CCC GAGAACATTAGTTCTC GATTGCAAAATC | 77 |
| MiddleB NEO_rs2073149 | TTTGATTTGAATAAACCAGAGAACTCTtCT GAG GAGAACATTAGTTCTC GATTGCAAAATC | 78 |
| MiddleB NEO_rs2862909 | TGTTGCTATCTTGCTtTTAGCATTTAGTGC GAGAACATTAGTTCTC GATTGCAAAATC | 79 |
| MiddleB NEO_rs1338945 | TCAGCGTTGAGTAATACcGTCTGCC GAGAACATTAGTTCTC GATTGCAAAATC | 80 |
| MiddleA NEO_rs12259813 | GCTAGAGAGATaATTGAGTGTCATCAGAAC TAGAT GAGAACATTAGTTCTC GTTAGCAATAAC | 81 |
| MiddleC NEO_rs354169 | CTGAGAACTTaGCATTAATTACCTTTTTTC ATGAGAAT CCTGTACACATACAGG GTTAGCAATAAC | 82 |
| MiddleC NEO_rs10230708 | ACTGCTGcAGGCGCCCTGT CCTGTACACATACAGG GTTAGCAATAAC | 83 |

### B. Type 2 NEOs

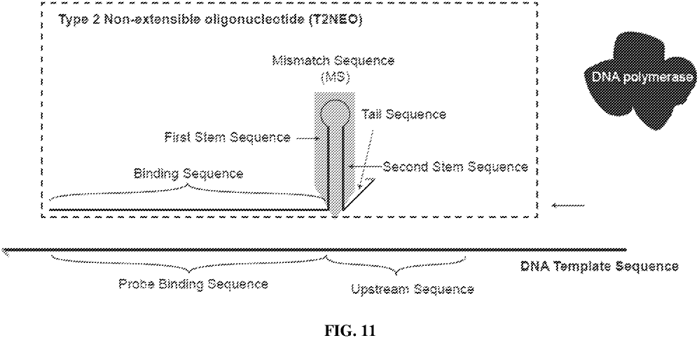

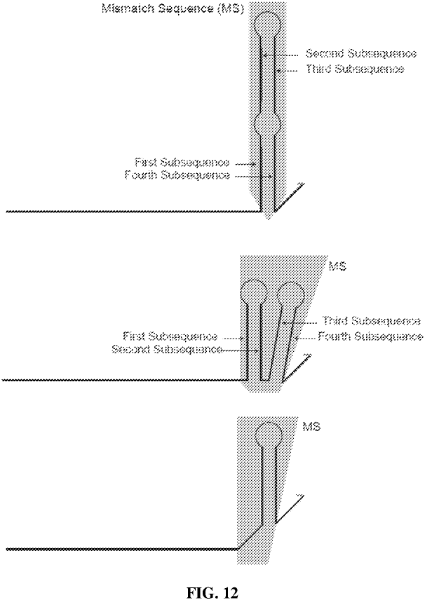

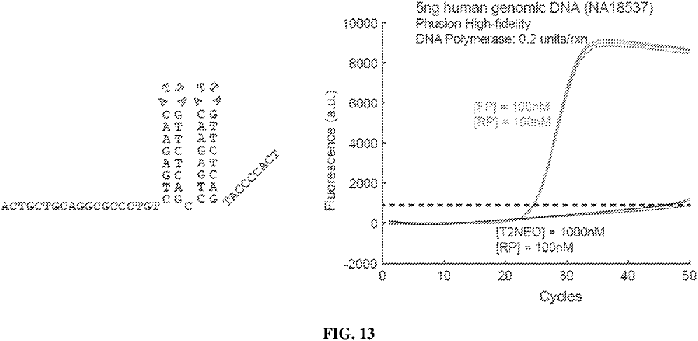

The Type 2 Non-Extensible Oligonucleotide (T2NEO) is similar to the T1NEO,except it does not comprise a Terminator Hairpin at the 3′ end. Instead, it comprises a Mismatch Sequence that comprises a hairpin sequence, and a 3′ Tail Sequence that does not comprise a hairpin (FIG. 11). Various embodiments of T2NEOs are shown in FIGS. 12&14. FIGS. 13&14 show sequences and experimental qPCR results demonstrating that T2NEOs are not effectively extended by the Phusion DNA polymerase.

### III. Applications of NEOs

### A. Blockers in Blocker Displacement Amplification (BDA) Allele Enrichment

BDA uses a non-extensible Blocker that has a sequence perfect matched against intended wildtype Template sequence. While the non-extensible oligonucleotide is bound to the Template, the Forward Primer cannot efficiently bind to the Template, because part of the Template sequence that binds to the NEO is also the subsequence that binds to the Forward Primer. In some embodiments, the subsequence of the Template that the Forward Primer binds to has a small number of nucleotides between 1 nucleotide and 20 nucleotides that is not encompassed within the subsequence of the Template that the NEO binds to.

If the Template sequence has even a single nucleotide sequence variant, the mismatch bubble formed between the Template and the NEO in the Binding Sequence causes a thermodynamic destabilization that results in the Forward Primer binding more favorably to the Template than NEO does. Taking T1NEO as an example, when there is a TC mismatch bubble formed due to sequence variant on Template, the T1NEO is displaced from the Template by Forward Primer. In some embodiments, the Forward Primer is then able to be extended by a DNA polymerase. In some embodiments, a mixture of wildtype Template and variant Template molecules are present in a Template sample, and the application of BDA with TEO to the sample results in the enrichment of the variant Templates over the wildtype Templates through selective amplification of the variant Templates. In some embodiments, the DNA polymerase is a thermostable DNA polymerase, and the amplification is achieved through polymerase chain reaction (PCR).

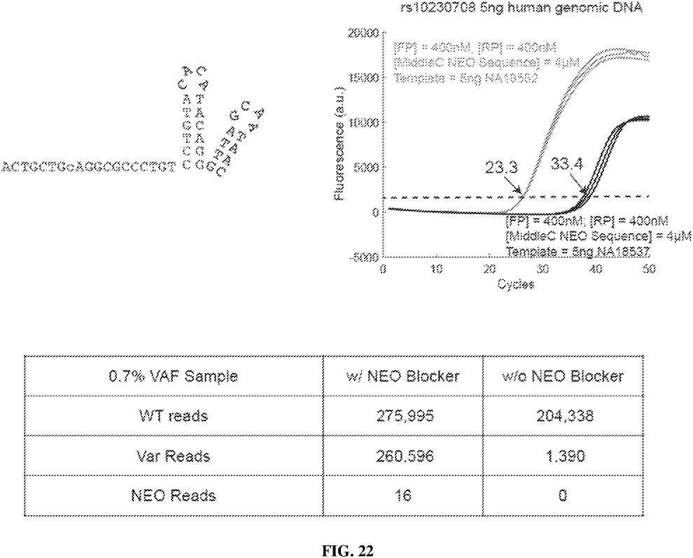

To demonstrate that the T1NEO that comprises a Middle Hairpin subtype can be applied in BDA, including qPCR and high-throughput sequencing, quantitative PCR (qPCR) and Next-Generation Sequencing (NGS) experiments were performed using the subtype NEO Sequences and their corresponding Forward Primers and Reverse Primers (FIG. 22). The top panel show experimental qPCR results. The Target DNA Template NA18562 human genomic DNA was enriched over the Background DNA Template NA18537 human genomic DNA. When MiddleC NEO Sequence was present, the NA18562 gDNA was amplified effectively with a Ct of 23.3, but the NA18537 gDNA was suppressed from amplification, with a Ct value of 33.4. As shown in the bottom panel, there was a summary of experimental NGS results using 80-plex PCR target enrichment. Here, 80 different forward primers and 80 different reverse primers were designed to 80 distinct regions of the human genome. Then 80 MiddleB NEO Sequence blockers with different Biological Sequence were designed to enrich variant amplicons. For the same 0.7% VAF sample, almost 200-fold more variant will be enriched by MiddleB NEO Sequence.

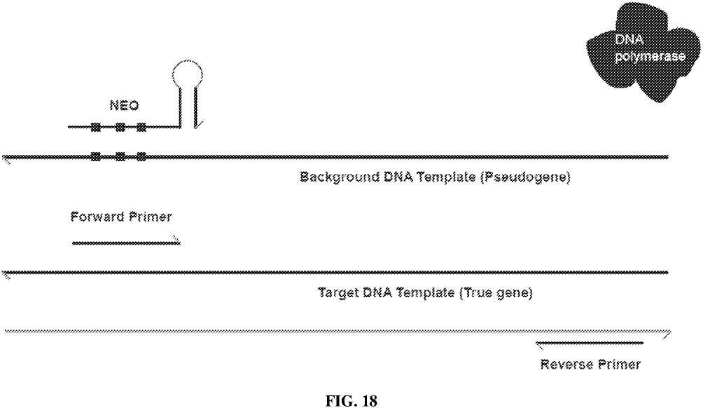

In some aspects, NEOs as BDA blockers can comprise a sequence that targets a pseudogene or other undesired genomic region and 3′ sequence or modification that prevents extension by DNA polymerase, thereby suppressing pseudogene amplification. For example, the NEO may be perfectly matched to pseudogene-specific sequences, and the Forward Primer is perfectly matched to corresponding true gene sequences (FIG. 18).

For BDA using a NEO as the blocker, BDA forward primer, NEO, reverse primer, DNA polymerase, dNTPs, and PCR buffer are mixed with the template sample for BDA amplification. Then BDA amplification is performed for 2, 3, 4, 5, 6, 7, 8, 9, 10, 11, 12, 13, 14, 15, 16, 17, 18, 19, 20, 21, 22, 23, 24, 25, 26, 27, 28, 29, 30, 2-30, 2-25, 2-23, 2-20, 2-15, 4-30, 4-25, 4-23, 4-20, 4-15, 6-30, 6-25, 6-23, 6-20, 6-15, 8-30, 8-25, 8-23, 8-20, 8-15, 10-30, 10-25, 10-23, 10-20, or 10-15 cycles of BDA amplification under conditions sufficient to achieve nucleic acid amplification.

As a BDA blocker, a NEO may include a first sequence having a target-neutral (i.e., wildtype) subsequence and a blocker variable (i.e., target) subsequence. In some aspects, the variable subsequence includes at least one nucleotide, at least two nucleotides, at least three nucleotides, at least four nucleotides, or at least five nucleotides. However, in some instances, the NEO may not include the blocker variable subsequence if the target nucleic acid to be detected is for the detection of an insertion. The NEO variable subsequence is flanked on its 3′ and 5′ ends by the target-neutral subsequence and is continuous with the target-neutral subsequence.

The BDA forward primer is sufficient to induce enzymatic extension of a template nucleic acid that is not bound by a NEO. The 3′ end of the BDA forward primer includes a sequence that overlaps with the 5′ end of the NEO. The portion of the NEO that overlaps with the 3′ end of the BDA forward primer consists only of target-neutral subsequence, thus the BDA forward primer may not include any sequence homologous with the blocker variable subsequence.

The sequences of the BDA forward primer and NEO may be rationally designed based on the thermodynamics of their hybridization to the target nucleic acid sequence (i.e., the sequence whose amplification or detection is desired, e.g., a SNP, an insertion, a deletion, or any other mutation) and the variant nucleic acid sequence (i.e., the sequence whose amplification is sought to be suppressed, e.g., a wild-type sequence). In some embodiments, the NEO is present in a significantly higher concentration than the BDA forward primer, so that the preponderance of the target and the variant nucleic acid sequences bind to the NEO before binding to primer. The BDA forward primer binds transiently to the BDA blocker-target or BDA blocker-variant molecules and possesses a probability for displacing the NEO in binding to the target or variant. Because the NEO sequence is specific to the variant target, its displacement from the variant is less thermodynamically favorable than its displacement from the wildtype. Thus, the non-allele-specific BDA forward primer amplifies the target sequence with higher yield/efficiency than it amplifies the wildtype sequence. WO2015/179339, which is incorporated herein by reference in its entirety, provides exemplary thermodynamic considerations that can be considered in designing BDA primer and NEO pairs.

In some aspects, the NEO and the BDA forward primer may be designed such that the binding of each oligonucleotide meets certain standard free energy of hybridization conditions. For example, the standard free energy of hybridization of the BDA forward primer to the template nucleic acid (ΔG°_{PT}) and the standard free energy of hybridization of the NEO to the template nucleic acid having the target sequence (ΔG°_{BT}) satisfies the following condition:

$$\text{+2} \text{kcal} / \text{mol} \geq Δ \text{G} {°}_{\text{PT}} \text{-} Δ \text{G} {°}_{\text{BT}} \geq \text{-8} \text{kcal} / \text{mol} .$$

In some aspects, NEO and the BDA forward primer may be designed such that ΔG°_{PT} -ΔG°_{BT} is between about +3 kcal/mol and about -10 kcal/mol, about +3 kcal/mol and about -9 kcal/mol, about +3 kcal/mol and about -8 kcal/mol, about +3 kcal/mol and about -7 kcal/mol, about +3 kcal/mol and about -6 kcal/mol, about +3 kcal/mol and about -5 kcal/mol, about +3 kcal/mol and about -4 kcal/mol, about +3 kcal/mol and about -3 kcal/mol, about +3 kcal/mol and about -2 kcal/mol, about +3 kcal/mol and about -1 kcal/mol, about +3 kcal/mol and about 0 kcal/mol, about +3 kcal/mol and about +1 kcal/mol, about +2 kcal/mol and about -10 kcal/mol, about +2 kcal/mol and about -9 kcal/mol, about +2 kcal/mol and about -8 kcal/mol, about +2 kcal/mol and about -7 kcal/mol, about +2 kcal/mol and about -6 kcal/mol, about +2 kcal/mol and about -5 kcal/mol, about +2 kcal/mol and about -4 kcal/mol, about +2 kcal/mol and about -3 kcal/mol, about +2 kcal/mol and about -2 kcal/mol, about +2 kcal/mol and about -1 kcal/mol, about +2 kcal/mol and about 0 kcal/mol, about +1 kcal/mole and about -10 kcal/mol, about +1 kcal/mol and about -9 kcal/mol, about +1 kcal/mol and about -8 kcal/mol, about +1 kcal/mol and about -7 kcal/mol, about +1 kcal/mol and about -6 kcal/mol, about +1 kcal/mol and about -5 kcal/mol, about +1 kcal/mol and about -4 kcal/mol, about +1 kcal/mol and about -3 kcal/mol, about +1 kcal/mol and about -2 kcal/mol, about +1 kcal/mol and about -1 kcal/mol, about 0 kcal/mole and about -10 kcal/mol, about 0 kcal/mol and about -9 kcal/mol, about 0 kcal/mol and about -8 kcal/mol, about 0 kcal/mol and about -7 kcal/mol, about 0 kcal/mol and about -6 kcal/mol, about 0 kcal/mol and about -5 kcal/mol, about 0 kcal/mol and about -4 kcal/mol, about 0 kcal/mol and about -3 kcal/mol, about 0 kcal/mol and about -2 kcal/mol, about -1 kcal/mole and about -10 kcal/mol, about -1 kcal/mol and about -9 kcal/mol, about -1 kcal/mol and about -8 kcal/mol, about -1 kcal/mol and about -7 kcal/mol, about -1 kcal/mol and about -6 kcal/mol, about -1 kcal/mol and about -5 kcal/mol, about -1 kcal/mol and about -4 kcal/mol, about -1 kcal/mol and about -3 kcal/mol, about -2 kcal/mole and about -10 kcal/mol, about -2 kcal/mol and about -9 kcal/mol, about -2 kcal/mol and about -8 kcal/mol, about -2 kcal/mol and about -7 kcal/mol, about -2 kcal/mol and about -6 kcal/mol, about -2 kcal/mol and about -5 kcal/mol, about -2 kcal/mol and about -4 kcal/mol, or about -2 kcal/mol and about -3 kcal/mol. In some aspects, BDA blocker and the BDA forward primer may be designed such that ΔG°_{PT} - ΔG°_{BT} is preferably between about -1 kcal/mol and about -4 kcal/mol at approximately 50° C., approximately 55° C., approximately 60° C., approximately 65° C., or approximately 70° C. in a buffer suitable for PCR.

In some aspects, the BDA forward primer may be designed such that the portion of the primer that does not hybridize with the NEO binding site has a standard free energy of hybridization (ΔG°₃) that is between about -4 kcal/mol and about -12 kcal/mol, about -4 kcal/mol and about -11 kcal/mol, about -4 kcal/mol and about -10 kcal/mol, about -4 kcal/mol and about -9 kcal/mol, about -4 kcal/mol and about -8 kcal/mol, about -4 kcal/mol and about -7 kcal/mol, about -4 kcal/mol and about -6 kcal/mol, about -5 kcal/mol and about -12 kcal/mol, about -5 kcal/mol and about -11 kcal/mol, about -5 kcal/mol and about -10 kcal/mol, about -5 kcal/mol and about -9 kcal/mol, about -5 kcal/mol and about -8 kcal/mol, about -5 kcal/mol and about -7 kcal/mol, about -6 kcal/mol and about -12 kcal/mol, about -6 kcal/mol and about -11 kcal/mol, about -6 kcal/mol and about -10 kcal/mol, about -6 kcal/mol and about -9 kcal/mol, about -6 kcal/mol and about -8 kcal/mol, about -7 kcal/mol and about -12 kcal/mol, about -7 kcal/mol and about -11 kcal/mol, about -7 kcal/mol and about -10 kcal/mol, about -7 kcal/mol and about -9 kcal/mol, about -8 kcal/mol and about -12 kcal/mol, about -8 kcal/mol and about -11 kcal/mol, about -8 kcal/mol and about -10 kcal/mol, about -9 kcal/mol and about -12 kcal/mol, about -9 kcal/mol and about -11 kcal/mol, or about -10 kcal/mol and about -12 kcal/mol.

Methods for the calculation of ΔG° values from sequence are known in the art. There exist different conventions for calculating the ΔG° of different region interactions. WO2015/179339, which is incorporated herein by reference in its entirety, provides exemplary energy calculations based on the nearest neighbor model. The calculation of ΔG°_{PT}, ΔG°_{PV}, ΔG°_{BT}, and ΔG°_{BV} from the primer sequence, blocker sequence, target sequence, variant sequence, operational temperature, and operational buffer conditions are known to those skilled in the art. The operational temperature may be about 20° C., about 25° C., about 30° C., about 35° C., about 40° C., about 45° C., about 50° C., about 55° C., about 60° C., about 65° C., or about 70° C. The operational buffer conditions may be buffer conditions suitable for PCR.

In some aspects, the BDA forward primer and NEO may each, individually, be from about 12-100, about 12-90, about 12-80, about 12-70, about 12-60, about 12-50, about 12-40, about 12-30, about 15-100, about 15-90, about 15-80, about 15-70, about 15-60, about 15-50, about 15-40, about 15-30, about 20-100, about 20-90, about 20-80, about 20-70, about 20-60, about 20-50, about 20-40, or about 20-30 nucleotides in length. In some aspects, the BDA forward primer and NEO may each, individually, be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

In some aspects, the portion of the BDA forward primer that hybridizes to the NEO binding site is between about 5-40 nucleotides, about 7-40, about 9-40, about 11-40, about 13-40, about 15-40, about 20-40, about 25-40, about 30-40, about 35-40, about 5-35, about 7-35, about 9-35, about 11-35, out 13-35, about 15-35, about 20-35, about 25-35, about 30-35, about 5-30, about 7-30, about 9-30, about 11-30, out 13-30, about 15-30, about 20-30, about 25-30, about 5-25, about 7-25, about 9-25, about 11-25, out 13-25, about 15-25, about 20-25, about 5-20, about 7-20, about 9-20, about 11-20, out 13-20, or about 15-20 nucleotides. In some aspects, the portion of the BDA forward primer that hybridizes to the NEO binding site is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

In some aspects, the concentration of the NEO is between about 2-10,000, about 2-9,000, about 2-8,000, about 2-7,000, about 2-6,000, about 2-5,000, about 2-4,000, about 2-3,000, about 2-2,000, about 2-1,000, about 2-900, about 2-800, about 2-700, about 2-600, about 2-500, about 2-400, about 2-300, about 2-200, about 2-150, about 2-100, about 2-90, about 2-80, about 2-70, about 2-60, about 2-50, about 2-40, about 2-30, about 2-20, about 2-10, about 4-10,000, about 4-9,000, about 4-8,000, about 4-7,000, about 4-6,000, about 4-5,000, about 4-4,000, about 4-3,000, about 4-2,000, about 4-1,000, about 4-900, about 4-800, about 4-700, about 4-600, about 4-500, about 4-400, about 4-300, about 4-200, about 4-150, about 4-100, about 4-90, about 4-80, about 4-70, about 4-60, about 4-50, about 4-40, about 4-30, about 4-20, about 4-10, about 6-10,000, about 6-9,000, about 6-8,000, about 6-7,000, about 6-6,000, about 6-5,000, about 6-4,000, about 6-3,000, about 6-2,000, about 6-1,000, about 6-900, about 6-800, about 6-700, about 6-600, about 6-500, about 6-400, about 6-300, about 6-200, about 6-150, about 6-100, about 6-90, about 6-80, about 6-70, about 6-60, about 6-50, about 6-40, about 6-30, about 6-20, about 6-10, about 10-10,000, about 10-9,000, about 10-8,000, about 10-7,000, about 10-6,000, about 10-5,000, about 10-4,000, about 10-3,000, about 10-2,000, about 10-1,000, about 10-900, about 10-800, about 10-700, about 10-600, about 10-500, about 10-400, about 10-300, about 10-200, about 10-150, about 10-100, about 10-90, about 10-80, about 10-70, about 10-60, about 10-50, about 10-40, about 10-30, about 10-20, about 20-10,000, about 20-9,000, about 20-8,000, about 20-7,000, about 20-6,000, about 20-5,000, about 20-4,000, about 20-3,000, about 20-2,000, about 20-1,000, about 20-900, about 20-800, about 20-700, about 20-600, about 20-500, about 20-400, about 20-300, about 20-200, about 20-150, about 20-100, about 20-90, about 20-80, about 20-70, about 20-60, about 20-50, about 20-40, about 20-30, about 40-10,000, about 40-9,000, about 40-8,000, about 40-7,000, about 40-6,000, about 40-5,000, about 40-4,000, about 40-3,000, about 40-2,000, about 40-1,000, about 40-900, about 40-800, about 40-700, about 40-600, about 40-500, about 40-400, about 40-300, about 40-200, about 40-150, about 40-100, about 40-90, about 40-80, about 40-70, about 40-60, or about 40-50 greater than the concentration of the BDA forward primer.

For multiplex BDA (mBDA) to simultaneously enrich potential target sequences at many groups of genetic loci, different BDA forward primers and NEOs are employed for each locus. These are all combined in solution simultaneously with the sample, a DNA polymerase, dNTPs, and buffers amenable for PCR. To prevent DNA-based inhibition of PCR, the total concentration of all oligo species can be kept under 50 micromolar. The length of the anneal/extend step of the PCR reaction is inversely proportional to the concentration of the lowest of the BDA forward primer species. To prevent excessively long protocols, it is recommended that all BDA forward primer concentrations be at least 100 picomolar. The concentration of each NEO species should be at least 2x that of its corresponding BDA forward primer species.

In addition to the standard design principles of single-plex BDA described above, oligo design for multiplex BDA (mBDA) requires further consideration to prevent undesired “primer dimer” species. Algorithms for mBDA sequence design should penalize candidate sequence sets when they are predicted to exhibit nonselective binding interactions. See, for example, WO 2019/164885, which is incorporated herein by reference in its entirety.

TABLE 2

Exemplary forward primers.

| Primer | Sequence | SEQ ID NO |
|---|---|---|
| FP_rs10230708 | ACCAATGGGAGTCACTGCTG | 84 |
| FP_rs10104396 | GAGGGGTATTAGAAGAATGACTATGTGA | 85 |
| FP_rs199032 | GCTCTTCCTCTCACATCTTTATTTAACC | 86 |
| FP_rs926850 | CAGAGTAAAATTTACTGCTCCGTCATAA | 87 |
| FP_rs17149369 | GGATTCCCTAAGCTCTTCAATATTGC | 88 |
| FP_rs869720 | CCTCATCTGTAAAGCAGGGAGAGA | 89 |
| FP_rs12478327 | ACTTCTGCCAACATTCAAATTCAGG | 90 |
| FP_rs2638145 | GGATGGGACTCCAATGCAAAACT | 91 |
| FP_rs2170091 | CATCTTGCTCTTCATAGATAGCTTCAGA | 92 |
| FP_rs2043583 | CCTGAATGTCAGTTTTGTTAGAGCAAC | 93 |
| FP_rs955456 | CAGACTTAATCAAAGCCCTTGAAAAGA | 94 |
| FP_rs966516 | CCTCCCATAGTGATTCTTATGAAGTCA | 95 |
| FP_rs354169 | AATGCTTTGCTTGCTGAGAACTT | 96 |
| FP_rs1898170 | AATGGGAAAACACATTTTAAGGGCA | 97 |
| FP_rs11247921 | CCACACTCTGCCTCTCATGGTAT | 98 |
| FP_rs1635718 | ACTTAAGAGGTCAACACAGATGAAAATTATC | 99 |
| FP_rs10510620 | TCCGCAAAACCTACAATCTCTGAA | 100 |
| FP_rs7104025 | TCAGATGCTTTAGGCTCATGAGTTA | 101 |
| FP_rs2246745 | CTCCTTGGAATCACCAACAAACAT | 102 |
| FP_rs3789806 | CTTGTATATAGACGGTAAAATAAACACCAAGA | 103 |
| FP_rs706714 | TGAAGCAGATGTTGAACAACAAGG | 104 |
| FP_rs1884444 | TTCCTGCTTCCAGACATGAATCA | 105 |
| FP_rs2510152 | ACCCAGGTGAGTTTTGTTTCACAT | 106 |
| FP_rs16754 | CTCTCTGCCTGCAGGATGTG | 107 |
| FP_rs206781 | CACTTCCTCCAGAAGGTCCAAAG | 108 |
| FP_rs28932178 | ACTAAGAGTGCAGAGCCTGGAA | 109 |
| FP_rs10186821 | GCGTTGTGCTGTCCATTGG | 110 |
| FP_rs10508599 | GGGTTAAAATCTTTTGCTTTCATATTGAGC | 111 |
| FP_rs10738578 | CCCGTTATATAAGAGGACATAATTGCAT | 112 |
| FP_rs10741037 | CACTTTATCAGACACAGTTATGTGCT | 113 |
| FP_rs10770674 | GCCCTATAGGTTTTCCTCCTACTGT | 114 |
| FP_rs10805227 | CTATCTGCAGGATTGTGTTCAATGTA | 115 |
| FP_rs10833604 | CTCTCTAGAGTGCAGATTGGTAGAA | 116 |
| FP_rs10964389 | CAAAGTTGATAAATTAAAGGACTAAGGCAC | 117 |
| FP_rs11015816 | CTGACCTAAGGCATGGGACTT | 118 |
| FP_rs11045749 | CATTCTGTCTGGGATGAGGTGAT | 119 |
| FP_rs1123828 | TGGAATCAAACATACTATGTGTCAAACA | 120 |
| FP_rs11708584 | GCGAAGTCATTTCGGTCCTCTTTAA | 121 |
| FP_rs12192635 | CCTCTGATTCCCAGACATAATGCT | 122 |
| FP_rs12213948 | TGAAAGACGTCACAGCAAGGT | 123 |
| FP_rs12259813 | TGTAGGAGAGATTGGGCTAGAGAG | 124 |
| FP_rs12541300 | ACAGAAACCAATTACCTATGAGGAGTAA | 125 |
| FP_rs12 681931 | GAAAGTGGCACAGAAACTCAGAC | 126 |
| FP_rs12782580 | GCATTAGATCATTTAACACACAAAACCCTAT | 127 |
| FP_rs1375977 | TGCTCCTAAAAGCACCCAGC | 128 |
| FP_rs1516755 | CTAACTTCCTAACTAAAACTTTACAGTGGA | 129 |
| FP_rs1524303 | GGATTTCACACCCATTAGAATAACTACTAT | 130 |
| FP_rs1667087 | CCTCTAGAAAAAATGGAGATTTGGGAAT | 131 |
| FP_rs16871316 | GGACTTTTTTGCTTTTTGACACCTTTAC | 132 |
| FP_rs16925478 | ACGTATTTCTAACTATAGTGAGTGCATTATG | 133 |
| FP_rs17560702 | ACATGTCCAAAGAGAGAAGTCGTAG | 134 |
| FP_rs1937037 | GCACGTAGATGAAATTGCCCCATA | 135 |
| FP_rs2215492 | GCCCCAAAGGTTACCCCATG | 136 |
| FP_rs2301720 | GTAGCCGCTTCTCTGTGAGTT | 137 |
| FP_rs2616187 | GGAAAATATGTCTAAAAAGGCTCTGGAG | 138 |
| FP_rs2710998 | GTTTGTTCTAAGGTTCATCTGGTGAT | 139 |
| FP_rs2807238 | GTGGGCTTACATGATTGGATTAACTT | 140 |
| FP_rs2874755 | TGTCCCACTTTTTACCTCCCTTC | 141 |
| FP_rs3813787 | GGGCTTCGGAATCGGACTTG | 142 |
| FP_rs4665582 | TGTGCTACGACAGAGCTAAGTAC | 143 |
| FP_rs4712476 | CCCCGGATGTCAGGGAATG | 144 |
| FP_rs611628 | CCAGGCACCACTGCTTTGT | 145 |
| FP_rs6452035 | GCAGAAAAAAATGATATCTGAATTCTGGAT | 146 |
| FP_rs6816854 | CCTTTTTCACTGTTATGAAATGTACTTTCTT | 147 |
| FP_rs6937778 | AGGATGCTGGGGCTTGC | 148 |
| FP_rs7003044 | GTAAAGTGCATGGGGTCAAGTC | 149 |
| FP_rs7032336 | TGAGAAGTCTAACAAGTTAAATTCAGGAC | 150 |
| FP_rs7816009 | GGTAGAATGTTAGTGACTATGTACAATTTCA | 151 |
| FP_rs7893462 | ACCTTGTCAAGAACCTAAATAGTGAGAA | 152 |
| FP_rs7902135 | CGTGGGCTAGTCAAGAATATAAAATGTTAG | 153 |
| FP_rs898476 | CCTATATAGACTAATTTACTTAAACATTTAAACCCCA | 154 |
| FP_rs9368431 | GGTTCAACTCTCAGTTTTATTAGTTGTGT | 155 |
| FP_rs9438621 | AGCATCGTGAGGTTCTGAAAAGA | 156 |
| FP_rs9466035 | CCTAACACCAGTTCTTCCTCCAC | 157 |
| FP_rs9466930 | TGTGTGGCTCAGTATACCACTTAG | 158 |
| FP_rs9973865 | GAAAAAAAAGGGTCTCATTAGGAATCATTAC | 159 |
| FP_rs4712498 | GTTTTTATATGTTAGTGTCCCCATGGTA | 160 |
| FP_rs2073149 | AGTGATCAGAAGGCTTTGATTTGA | 161 |
| FP_rs2862909 | GCACATCATACATTATTTCTGTTGCTAT | 162 |
| FP_rs1338945 | GAAATATTGCTGGGGTCAGCG | 163 |

TABLE 3

Exemplary reverse primers

| Primer | Sequence | SEQ ID NO |
|---|---|---|
| RP_rs10230708 | TAAGTGGAAAGAACTGGGGTGTC | 164 |
| RP_rs10104396 | ACATGGTTAGATATTAGCCTGACCTATG | 165 |
| RP_rs199032 | GCAGCCAAGTGTGAAAGTATTGA | 166 |
| RP_rs926850 | TGATGTTGAGTTGAGACAGGTTACA | 167 |
| RP_rs17149369 | AAATGTAGT TCTAT TATGGTCAGCACAC | 168 |
| RP_rs869720 | AGTATCCCCAAAAGGTTGCAGAT | 169 |
| RP_rs12478327 | GTGCAAGCTGGAGGCACT | 170 |
| RP_rs2638145 | ACAGGAAAAGAAACTAAAATTGTACCCTT | 171 |
| RP_rs2170091 | GAAGCCAGATCTCAAAGTGTCCT | 172 |
| RP_rs2043583 | GTTATTGGGAATGCTATGAAAGAGACA | 173 |
| RP_rs955456 | AGAACTCATTTCCTTATAGCTGAAGAACT | 174 |
| RP_rs966516 | GCAGACACTTAGGATGTTTCCAGT | 175 |
| RP_rs354169 | GAGCCTTAGTTCCTCCATCAGTAAA | 176 |
| RP_rs1898170 | AAATTTACGTTGGTAATTGGGTCTTGT | 177 |
| RP_rs11247921 | CACAGAGGTGACAGAACACAGT | 178 |
| RP_rs1635718 | TAGTTATTCATGGTGGGAAGGCAA | 179 |
| RP_rs10510620 | AAAAGATAATGTTCTTGTTTATATGCCCTTG | 180 |
| RP_rs7104025 | TACAGCAACTCACAAACTAATGACTCT | 181 |
| RP_rs2246745 | GGCTGCGATGAGACAGGAA | 182 |
| RP_rs3789806 | AGGCACCAGAAGTCATCAGAATG | 183 |
| RP_rs706714 | GACCAAGC T T T TAT GCACCACA | 184 |
| RP_rs1884444 | TGAAAGATAGCAATAGATACATAAAACACCA | 185 |
| RP_rs2510152 | TGAAACCACATACACACAAATTCACT | 186 |
| RP_rs16754 | CTTCCTGCTGTGCATCTGTAAGT | 187 |
| RP_rs206781 | AAAAAGAAGAAACGGAAGGCAGAG | 188 |
| RP_rs28932178 | TGCTGCCCCACCCTTTATTAAC | 189 |
| RP_rs10186821 | CCTATTGGAAGAACCTGCCAGAA | 190 |
| RP_rs10508599 | TGCAAAATGAAGCACAGCCC | 191 |
| RP_rs10738578 | GCAGATGGAAAATACTTGGGAAAAAAAT | 192 |
| RP_rs10741037 | GCAAAAATTACTATACCGACTTTAATAACGAAA | 193 |
| RP_rs10770674 | ACTCATTGTAGGCTGAACCTTGG | 194 |
| RP_rs10805227 | TGTATTGAGCATTTAGCACATGCC | 195 |
| RP_rs10833604 | CAATTTCCAAGACAGAAGCACTCC | 196 |
| RP_rs10964389 | ACTTACTGAGCACATGGCCTG | 197 |
| RP_rs11015816 | GGAGAGGGTGAGAAGTTGCAC | 198 |
| RP_rs11045749 | GGCAAAGACATTTTTCCAAGGAAGATAT | 199 |
| RP_rs1123828 | CACTGCCAGCTTGTGCCT | 200 |
| RP_rs11708584 | GCCCTAAATCCTAAATGAAATTGGCA | 201 |
| RP_rs12192635 | AGAGGAGAAATAGATGTAGCTGCC | 202 |
| RP_rs12213948 | AATCCAGTGACATTCTTTAAACTGTCTT | 203 |
| RP_rs12259813 | GCTGAGCTGTCACATCACTTCA | 204 |
| RP_rs12541300 | GCTGTGTAGCTTGGCAAATTAACTA | 205 |
| RP_rs12681931 | GCACTCTTGGGTAACAGGCTTT | 206 |
| RP_rs12782580 | CCATGCCCAGCCTGGC | 207 |
| RP_rs1375977 | TGGCTCCTCATAAGTTATGCAGATTT | 208 |
| RP_rs1516755 | CAGTAGGATTGGCTTTATCAAAGAGATC | 209 |
| RP_rs1524303 | ACCATAATGTTTTCCATAGAAGATGCAC | 210 |
| RP_rs1667087 | GGTTCTGTACTGAAGTAAAAATCTCATACTAT | 211 |
| RP_rs16871316 | GGCAAAGAAACATGGCAGAAATATCATA | 212 |
| RP_rs16925478 | CCTTTGGCATTTTGGTCAAGATTGT | 213 |
| RP_rs17560702 | GGGGGAAAATGGTTTCTTAGGATGA | 214 |
| RP_rs1937037 | CTCCCATTTTTCTAAGACATTTTTTTTTCTC | 215 |
| RP_rs2215492 | AGCATGCCGCCCTTGG | 216 |
| RP_rs2301720 | TCACAGGTCAAAATTATGAGTTCTTCG | 217 |
| RP_rs2616187 | TGAGAGTGTGCAAGTCACTTGT | 218 |
| RP_rs2710998 | GCAGGCAGCATGTATCCCAG | 219 |
| RP_rs2807238 | GTTTAATGGACAGTAGATGCTAAATTCTAGA | 220 |
| RP_rs2874755 | CGCCATAGTTAGCCGCTTCC | 221 |
| RP_rs3813787 | TGAGCCTCGGTCTCTACCTG | 222 |
| RP_rs4665582 | CCTTTAAGGCCCAGCAACTG | 223 |
| RP_rs4712476 | GGGTGACCTTTCCCTTTTGATGA | 224 |
| RP_rs611628 | TGTGTGTGAAAGCACTTTATAAACCA | 225 |
| RP_rs6452035 | CTATCCTCAGAATTTTCCATTGATACTAGAAATA | 226 |
| RP_rs6816854 | GAGTGTCTCCCAAACAAGGATCA | 227 |
| RP_rs6937778 | ACAGCCATCAGATATCCAGCAG | 228 |
| RP_rs7003044 | ACTTCGAGAATTGACTCTAAGTGGT | 229 |
| RP_rs7032336 | AATTTAGCTTCCTTGAGGATAGAAGTAAC | 230 |
| RP_rs7816009 | CCCGGCCACCCATACAG | 231 |
| RP_rs7893462 | GAAAACTACCTTAAACTATGTGAGAAAGAAC | 232 |
| RP_rs7902135 | ACCCTCACTAATCTTTTTCTGTTTGTTT | 233 |
| RP_rs898476 | GTTTTTCTCCCAGCTGTAAAAGCA | 234 |
| RP_rs9368431 | GCTTTAGTTTCTTTGCATATTTTCTGCAATA | 235 |
| RP_rs9438621 | AGCTGATCTGCAAGGTCTATTTGA | 236 |
| RP_rs9466035 | TGGGCTCAAGTGATCCACCTA | 237 |
| RP_rs9466930 | GTAAAGAGAAGGGCTACCAGGATTA | 238 |
| RP_rs9973865 | CCCTATGCCTGGGATACTTCCTT | 239 |
| RP_rs4712498 | AGACCGAACTTGTTGCGAATCA | 240 |
| RP_rs2073149 | TCTTTTATCCAGTTGCCTCTATTTTACAC | 241 |
| RP_rs2862909 | TTCAAAAACCCATTCATACAAGGTCAG | 242 |
| RP_rs1338945 | AGGAGAGGGAGGAGCATGG | 243 |

### B. Hybrid-Capture Probes

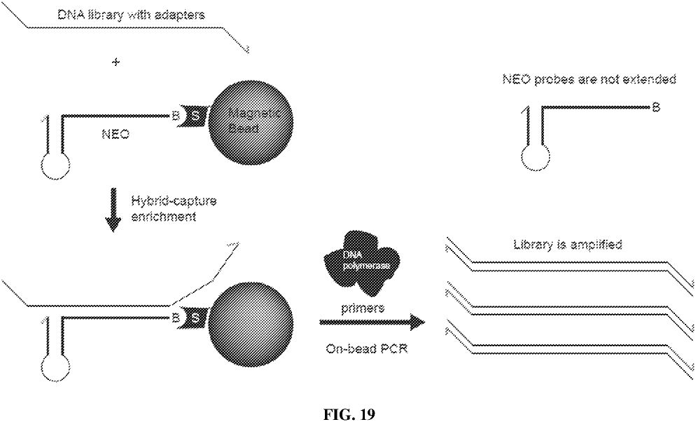

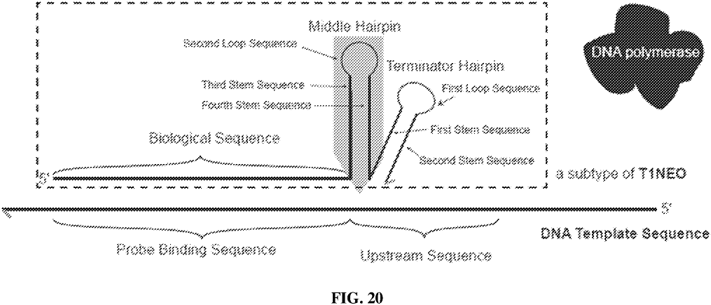

In another embodiment, NEOs can be used as hybrid-capture probes for target enrichment in NGS applications (FIG. 19). A hybrid-capture probe comprises a nucleic acid sequence which is capable of hybridizing to unique region(s) within a target nucleic acid and being captured onto a solid phase. Doing so with an NEO allows on-bead PCR amplification after hybrid-capture enrichment without consideration for the confounding impacts of probe extension since the NEO probes are non-extensible. For this, the NEO may be functionalized with a 5′-biotinylation to allow for binding to streptavidin-coated magnetic beads. These NEOs provide a means to selectively hybridize and capture DNA molecules corresponding to genes of interest.

Various uses for hybrid-capture probes are well known to the skilled artisan and may be applied to the detection and discrimination of a variety of mutations including, but not limited to insertions, deletions, inversions, repeated sequences, and multiple as well as single nucleotide polymorphisms (SNPs). In some embodiments, a panel of NEOs targeting various sequences may be used as hybrid-capture probes. For example, a panel of NEOs may be designed to distinguish different human genomes based on SNP signature. In some embodiments, a test sample may contain a complex mixture of nucleic acids, of which the target nucleic acid may correspond to a gene of interest contained in total human genomic DNA or RNA or a portion of the nucleic acid sequence of a pathogenic organism that is a minor component of a clinical sample.

### IV. Further Processing of Target Nucleic Acids

### A. Amplification of DNA

A number of template-dependent processes are available to amplify the nucleic acids present in a given template sample. One of the best known amplification methods is the polymerase chain reaction (referred to as PCR™) which is described in detail in U.S. Pat. Nos. 4,683,195, 4,683,202, and 4,800,159 and in Innis et al., 1990, each of which is incorporated herein by reference in their entirety. Briefly, two synthetic oligonucleotide primers, which are complementary to two regions of the template DNA (one for each strand) to be amplified, are added to the template DNA (that need not be pure), in the presence of excess deoxynucleotides (dNTP’s) and a thermostable polymerase, such as, for example, Taq (Thermus aquaticus) DNA polymerase. In a series (typically 30-35) of temperature cycles, the target DNA is repeatedly denatured (around 90° C.), annealed to the primers (typically at 50-60° C.) and a daughter strand extended from the primers (72° C.). As the daughter strands are created they act as templates in subsequent cycles. Thus, the template region between the two primers is amplified exponentially, rather than linearly.

### B. Sequencing of DNA

Methods are also provided for the sequencing of the library of adaptor-linked fragments. Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing.

The nucleic acid library may be generated with an approach compatible with Illumina sequencing such as a Nextera™ DNA sample prep kit, and additional approaches for generating Illumina next-generation sequencing library preparation are described, e.g., in Oyola et al. (2012). In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.). Additional methods for next-generation sequencing methods, including various methods for library construction that may be used with embodiments of the present invention are described, e.g., in Pareek (2011) and Thudi (2012).

In particular aspects, the sequencing technologies used in the methods of the present disclosure include the HiSeq™ system (e.g., HiSeq™ 2000 and HiSeq™ 1000), the NextSeq™ 500, and the MiSeq™ system from Illumina, Inc. The HiSeq™ system is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. The MiSeq™ system uses TruSeq™, Illumina’s reversible terminator-based sequencing-by-synthesis.

Another example of a DNA sequencing technique that can be used in the methods of the present disclosure is 454 sequencing (Roche) (Margulies et al., 2005). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil- water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection— no scanning, no cameras, no light— each nucleotide incorporation is recorded in seconds.

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

A further sequencing platform includes the CGA Platform (Complete Genomics). The CGA technology is based on preparation of circular DNA libraries and rolling circle amplification (RCA) to generate DNA nanoballs that are arrayed on a solid support (Drmanac et al. 2009). Complete genomics’ CGA Platform uses a novel strategy called combinatorial probe anchor ligation (cPAL) for sequencing. The process begins by hybridization between an anchor molecule and one of the unique adapters. Four degenerate 9-mer oligonucleotides are labeled with specific fluorophores that correspond to a specific nucleotide (A, C, G, or T) in the first position of the probe. Sequence determination occurs in a reaction where the correct matching probe is hybridized to a template and ligated to the anchor using T4 DNA ligase. After imaging of the ligated products, the ligated anchor-probe molecules are denatured. The process of hybridization, ligation, imaging, and denaturing is repeated five times using new sets of fluorescently labeled 9-mer probes that contain known bases at the n + 1, n + 2, n + 3, and n + 4 positions.

### V. Kits

The technology described herein includes kits comprising non-extensible oligonucleotides as disclosed herein. Exemplary kits include qPCR kits, Sanger kits, NGS panels, and nanopore sequencing panels. A “kit” refers to a combination of physical elements. For example, a kit may include, for example, one or more components such as nucleic acid primers, nucleic acid blockers, enzymes, reaction buffers, an instruction sheet, and other elements useful to practice the technology described herein. These physical elements can be arranged in any way suitable for carrying out the invention.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow molded plastic containers into which the desired vials are retained. A kit will also include instructions for employing the kit components as well the use of any other reagent not included in the kit. Instructions may include variations that can be implemented.

### VL Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 - Quantitative PCR (qPCR) Reaction Protocols and Conditions

For experimental results presented here, the final concentration of the Forward Primer and the Reverse Primer are each 100 nM or 400 nM, as noted, in 10 µL of reaction mixture. The final concentration of the NEO is 100 nM, 1000 nM, or 4000 nM, as marked in the figures. Unless otherwise noted, the Phusion Hi-Fi DNA polymerase, syto 13-interacting dye, and reagents needed for Phusion were used for all qPCR experiments (New England Biolabs, Inc). Input DNA is typically 5 ng of NA18537 or NA18562 human genomic DNA (Coriell Cell Repositories). Thermal cycling and fluorescence measurement were performed using a Bio-Rad CFX96 qPCR instrument. The thermal cycling protocol was as follows: 1. 98° C. 30 seconds; 2. 50 cycles of (98° C. for 10 seconds, 60° C. for 30 seconds, 72° C. for 30 seconds).

The same protocols and conditions were used for Blocker Displacement Amplification (BDA) qPCR experiments using NEO as the Blocker.

We used the PowerUp SYBR DNA Polymerase Mastermix (Thermo Fisher) for all Taq-based experiments. Input DNA is typically 5 ng of NA18537 human genomic DNA (Coriell Cell Repositories). Thermal cycling and fluorescence measurement were performed using a Bio-Rad CFX96 qPCR instrument. The thermal cycling protocol is as follows: 1. 95° C. 3 minutes; 2. 50 cycles of (95° C. for 10 seconds, 60° C. for 30 seconds).

### Example 2 - Next Generation Sequencing (NGS) Reaction Protocols and Conditions

The data for the NGS experiments summarized in FIG. 22 were collected using an Illumina MiSeq instrument and a MiSeq v2 paired-end 150+150 cycle kit. Each library used 25 ng input DNA in 50 uL reaction mixture. The library preparation process is briefly summarized below:
1a. For libraries without Blocker, perform 13 cycles BDA PCR (98° C. for 10 seconds, 60° C. for 5 minutes, 72° C. for 2 minutes) using Phusion 2X MasterMix (New England Biolabs, Inc), using 15 nM primer per plex.
1b. For libraries with Blocker, perform 23 cycles for BDA PCR (98° C. for 10 seconds, 60° C. for 5 minutes, 72° C. for 2 minutes) Phusion 2X MasterMix (New England Biolabs, Inc), using 15 nM primer per plex and 150 nM blocker per plex.
2. Perform DNA purification using 1.8x SPRI beads.
3. Perform 2 cycles adapter PCR (98° C. for 10 seconds, 60° C. for 5 minutes, 72° C. for 2 minutes) using Phusion 2X MasterMix (New England Biolabs, Inc), using 15 nM primer per plex.
4. Perform DNA purification using 1.6x SPRI beads.
5a. For libraries without Blocker, perform 15 cycles index PCR (98° C. for 10 seconds, 60° C. for 60 seconds, 72° C. for 60 seconds) using Phusion 2X MasterMix (New England Biolabs, Inc), using 500 nM index primers.
5b. For libraries with Blocker, perform 12 cycles for index PCR (98° C. for 10 seconds, 60° C. for 60 seconds, 72° C. for 60 seconds) Phusion 2X MasterMix (New England Biolabs, Inc), using 500 nM index primers.
6a. For libraries without Blocker, perform DNA purification using 0.6x+0.3x SPRI beads.
6b. For libraries with Blocker, perform DNA purification using 0.7x+0.3x SPRI beads.

### Example 3 - NGS Bioinformatic Analysis Methods

The method for analyzing NGS reads from NGS FASTQ files is summarized below:
1. Trim adapters sequences from each read.
2. Count the number of insert reads that perfectly match the 10nts before and after SNP loci of wildtype amplicon (WT Reads) or 10nts before and after SNP loci of variant amplicon (Var Reads). Any degenerate nucleotides in the reads, such as N, are considered mismatched and do not contribute to WT Reads or Var Reads.
3. Count the number of insert reads that perfectly match the Middle Hairpin sequence, Terminator Hairpin sequence or the complete sequence of Middle Hairpin sequence and Terminator Hairpin sequence. The total number will be counted as NEO Reads.

### Example 4 - Fold-Enrichment Analysis and VAF Quantitation

The fold-enrichment (EF) for a variant Template is defined as the relative amplification of the variant Template over the corresponding wildtype Template. In general, larger number of PCR cycles with ACE result in larger EF values. In an NGS library setting, the values of VRF, EF, and variant allele frequency (VAF) satisfy the following equations:

$$\text{VRF} = \left(\text{VAF * EF}\right) / \left(\text{VAF * EF +} \left(\text{1-VAF}\right)\right)$$

$$\text{VAF =} \left(\text{VRF}\right) / \left(\text{VRF *} \left(\text{1-EF}\right) \text{+ EF}\right)$$

$$\text{EF =} \left(\text{VRF *} \left(\text{VAF-1}\right)\right) / \left(\text{VAF *} \left(\text{VRF-1}\right)\right)$$

Given the known values of any two of the three variables, the last variable can be calculated. Thus, during initial calibration experiments, VRF and VAF from known samples can be used to calculate EF. Afterwards, when running NGS on samples with unknown VAFs, VRF and EF can be used to calculate the value of VAF.

### Example 5 - T1NEOs and T2NEOs Are Not Effectively Extended by High Fidelity DNA Polymerases

The 3′->5′ exonuclease activity of high-fidelity DNA polymerases is a critical feature that enables these enzymes to be used for detection and quantitation of mutations with low variant allele frequencies (VAFs), such as somatic mutations in tumor tissue or cell-free DNA. The 3′->5′ exonuclease activity allows kinetic proofreading, whereby incorrectly incorporated DNA nucleotides at the 3′ end of a growing amplicon can be removed, and enables DNA polymerases such as Phusion and Q5 to exhibit misincorporation error rates that are between 20- and 200-fold lower than Taq-based DNA polymerases. However, this 3′->5′ exonuclease activity also renders it challenging to design DNA probes and blockers that are not intended to be enzymatically extended (FIG. 9). Even many 3′ chemical modifications that prevent Taq extension are not effective at preventing extension after 3′->5′ exonuclease activity. FIG. 10 shows a series of DNA oligos with and without 3′ chemical modifications that are less effective at preventing enzymatic extension by DNA polymerases with 3′->5′ exonuclease activity.

To demonstrate that the T1NEOs cannot be effectively extended by DNA polymerases, including by high fidelity DNA polymerases with 3′->5′ exonuclease activity, a number of quantitative PCR (qPCR) experiments were performed using a T1NEO and a Reverse Primer (5′-ACATGGTTAGATATTAGCCTGACCTATG-3′; SEQ ID NO: 165) (FIGS. 6-8). No qPCR amplification or very late amplification indicated that the T1NEO was not enzymatically extended. In contrast, using a Forward Primer (5′-GAGGGGTATTAGAAGAATGACTATGTGA-3′; SEQ ID NO: 85) with similar sequence to the T1NEO, but lacking the Terminator Hairpin, showed effective qPCR amplification and detection, indicating that the primer designs, DNA polymerases, and DNA input sample are all compatible with PCR amplification. In these experiments, the Syto-13 intercalating DNA dye, which produces fluorescence nonspecifically to buildup of dsDNA amplicons, was used.

To demonstrate that the T1NEO having a Middle Hairpin cannot be effectively extended by DNA polymerases, including by high fidelity DNA polymerases with 3′->5′ exonuclease activity, a number of quantitative PCR (qPCR) experiments were performed using three listed sequences (MiddleA (SEQ ID NO: 81), MiddleB (SEQ ID NO: 2) or MiddleC (SEQ ID NO: 82) NEO Sequence) and their corresponding Reverse Primers ((SEQ ID NOs: 204, 165, and 176, respectively; FIG. 21). No qPCR amplification or very late amplification indicates that this subtype was not enzymatically extended. In contrast, using a Forward Primer with similar sequence to the NEO Sequences, but lacking the Middle Hairpin and Terminator Hairpin, showed effective qPCR amplification and detection, indicating that the primer designs, DNA polymerases, and DNA input sample are all compatible with PCR amplification. In these experiments, the Syto-13 intercalating DNA dye, which produces fluorescence nonspecifically to buildup of dsDNA amplicons, was used.

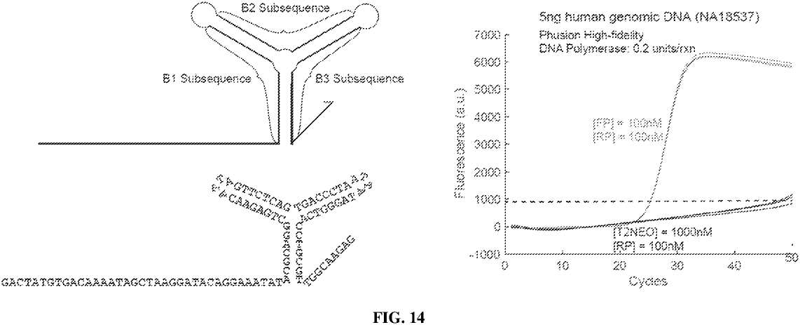

To demonstrate that both a T2NEO with two hairpins in the MS and a 9nt Tail Sequence (FIG. 13) as well as a T2NEO with a branched hairpin structure (FIG. 14) cannot be effectively extended by high fidelity DNA polymerases, quantitative PCR (qPCR) was applied to a NA18537 human genomic DNA templates using the Phusion high-fidelity DNA polymerase with 3′->5′ exonuclease activity and using Syto-13 intercalating dye. No observable PCR amplification occurred even when a 10-fold higher concentration of T2NEO was used (FIGS. 13&14). In contrast, a forward primer and a reverse primer were able to effectively perform qPCR amplification (FIGS. 13&14). Thus, T2NEO cannot be enzymatically extended even by DNA polymerases with 3′->5′ exonuclease activity. The slow and late fluorescence increase in the T2NEO traces may be due to RP primer dimer or nonspecific amplification on the genome.

### Example 6 - NEOs as BDA Blockers

BDA uses a non-extensible Blocker that has a sequence perfectly matched against an intended wildtype Template sequence. In BDA, the non-extensible Blocker oligonucleotide overlaps in sequence with a Forward Primer, so that the Blocker and Forward Primer compete in binding to DNA templates. While the non-extensible oligonucleotide is bound to the Template, the Forward Primer cannot efficiently bind to the Template, because part of the Template sequence that binds to the NEO is also the subsequence that binds to the Forward Primer. In some embodiments, the subsequence of the Template that the Forward Primer binds to has a small number of nucleotides, between 1 nucleotide and 20 nucleotides, that is not encompassed within the subsequence of the Template to which the NEO binds.

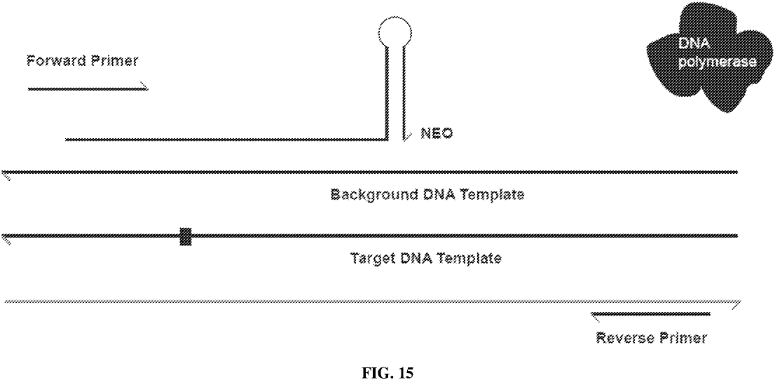

If the Template sequence has even a single nucleotide sequence variant, the mismatch bubble formed between the Template and the NEO in the Binding Sequence causes a thermodynamic destabilization that results in the Forward Primer binding more favorably to the Template than the NEO binding to the Template (FIG. 15). Taking a T1NEO as an example, when there is a TC mismatch bubble formed due to sequence variant on Template, the T1NEO is displaced from the Template by the Forward Primer. In some embodiments, the Forward Primer is then able to be extended by a DNA polymerase. In some embodiments, a mixture of wildtype Template and variant Template molecules are present in a Template sample, and the application of BDA with NEO to the sample results in the enrichment of the variant Templates over the wildtype Templates through selective amplification of the variant Templates (FIG. 15). In some embodiments, the DNA polymerase is a thermostable DNA polymerase, and the amplification is achieved through polymerase chain reaction (PCR).

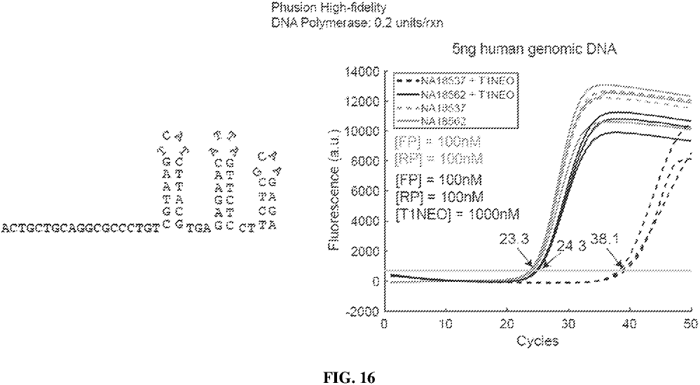

To demonstrate this using a T1NEO, NA19537 human genomic DNA was used as the wildtype Template, and NA18562 human genomic DNA was used as the variant Template. A T1NEO was designed to cover the rs10230708 single nucleotide polymorphism (SNP) locus, in which NA18537 is homozygous for the G allele on the Template Sequence corresponding to the C nucleotide on the T1NEO, and NA18562 is homozygous for the T allele, which is mismatched against T1NEO. In the absence of the T1NEO, both NA18537 and NA18562 amplified effectively with cycle threshold (Ct) values of about 23.3 (FIG. 16). When the T1NEO was present, the NA18562 gDNA was still amplified effectively with a Ct of 24.3, but the NA18537 gDNA was suppressed from amplification, with a Ct value of 38.1 (FIG. 16).

To demonstrate that the T1NEO having a Middle Hairpin can be applied in BDA, including qPCR and high-throughput sequencing, quantitative PCR (qPCR) and Next-Generation Sequencing (NGS) experiments were performed using this subtype NEO Sequences and their corresponding Forward Primers and Reverse Primers (FIG. 22). The top panel show experimental qPCR results (using a NEO according to SEQ ID NO: 83; forward primer according to SEQ ID NO: 84, and reverse primer according to SEQ ID NO: 164. The Target DNA Template NA18562 human genomic DNA was enriched over the Background DNA Template NA18537 human genomic DNA. When MiddleC NEO Sequence was present, the NA18562 gDNA is amplified effectively with a Ct of 23.3, but the NA18537 gDNA was suppressed from amplification, with a Ct value of 33.4. As shown in the bottom panel, there was a summary of experimental NGS results using 80-plex PCR target enrichment. Here, 80 different forward primers and 80 different reverse primers were designed to 80 distinct regions of the human genome. Then 80 MiddleB NEO Sequence blockers with different Biological Sequence were designed to enrich variant amplicons. For the same 0.7% VAF sample, almost 200-fold more variant will be enriched by MiddleB NEO Sequence.

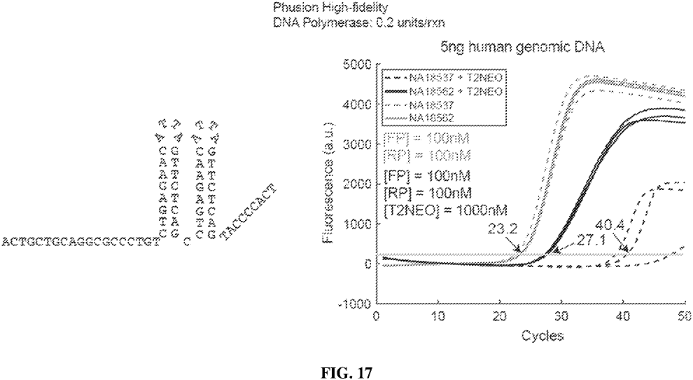

To demonstrate this using a T2NEO, NA19537 human genomic DNA was again used as the wildtype Template, and NA18562 human genomic DNA was again used as the variant Template. A T2NEO was designed to cover the rs10230708 single nucleotide polymorphism (SNP) locus, in which NA18537 is homozygous for the G allele on the Template Sequence corresponding to the C nucleotide on the T2NEO, and NA18562 is homozygous for the T allele, which is mismatched against T2NEO. In the absence of the T2NEO, both NA18537 and NA18562 amplified effectively with cycle threshold (Ct) values of about 23.2 (FIG. 17). When the T2NEO was present, the NA18562 gDNA was still amplified effectively with a Ct of 27.1, but the NA18537 gDNA was suppressed from amplification, with a Ct value of 40.4 (FIG. 17).

In some aspects, NEOs as BDA blockers can comprise a sequence that targets a pseudogene or other undesired genomic region and 3′ sequence or modification that prevents extension by DNA polymerase, thereby suppressing pseudogene amplification. For example, the NEO may be perfectly matched to pseudogene-specific sequences, and the Forward Primer is perfectly matched to corresponding true gene sequences (FIG. 18).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising a DNA template, a DNA polymerase, and a non-extensible oligonucleotide, wherein the DNA template comprises continuously from 5′ to 3′ an upstream sequence and a probe binding sequence, wherein the non-extensible oligonucleotide comprises from 5′ to 3′:
a binding sequence that is at least 70% identical to the reverse complement of the probe binding sequence of the DNA template, and
a terminator hairpin, positioned at the 3′-end of the non-extensible oligonucleotide, that comprises:
a first stem sequence,
a second stem sequence, wherein the second stem sequence is the reverse complement of the first stem sequence, and
a first loop sequence positioned between the first stem sequence and the second stem sequence.

2. The composition of claim 1, wherein the binding sequence of the non-extensible oligonucleotide is between 10 and 300 nucleotides long.

3. The composition of claim 1 or 2, wherein the terminator hairpin of the non-extensible oligonucleotide is not the reverse complement of the upstream sequence of the DNA template.

4. The composition of claim 3, wherein the terminator hairpin of the non-extensible oligonucleotide is unable to hybridize to the upstream sequence of the DNA template.

5. The composition of any one of claims 1-4, wherein the first stem sequence of the terminator hairpin is between 3 and 8 nucleotides long.

6. The composition of any one of claims 1-5, wherein the first stem sequence of the terminator hairpin is four nucleotides long.

7. The composition of any one of claims 1-6, wherein the second stem sequence of the terminator hairpin is between 3 and 8 nucleotides long.

8. The composition of any one of claims 1-7, wherein the second stem sequence of the terminator hairpin is four nucleotides long.

9. The composition of any one of claims 1-8, wherein the first stem sequence and the second stem sequence of the terminator hairpin are both 4 nucleotides long.

10. The composition of any one of claims 1-9, wherein the terminator hairpin has an adenine nucleotide as its 3′-most nucleotide.

11. The composition of any one of claims 1-10, wherein the first stem sequence is 5′-TCTC-3′ and the second stem sequence is 5′-GAGA-3′.

12. The composition of any one of claims 1-9, wherein the first stem sequence is 5′-GTTC-3′ and the second stem sequence is 5′-GAAC-3′.

13. The composition of any one of claims 1-12, wherein the first loop sequence of the terminator hairpin is between 3 and 10 nucleotides long.

14. The composition of any one of claims 1-13, wherein the first loop sequence of the terminator hairpin is four nucleotides long.

15. The composition of any one of claims 1-14, wherein the first loop sequence is 5′-GCAA-3′.

16. The composition of any one of claims 1-15, wherein the non-extensible oligonucleotide further comprises a middle hairpin positioned between the binding sequence and the terminator hairpin, the middle hairpin comprising:
a third stem sequence,
a fourth stem sequence, wherein the fourth stem sequence is the reverse complement of the third stem sequence, and
a second loop sequence positioned between the third stem sequence and the fourth stem sequence.

17. The composition of claim 16, wherein the 3′-most nucleotide of the terminator hairpin is a cytosine.

18. The composition of claim 16 or 17, wherein the first stem sequence of the terminator hairpin and the second stem sequence of the terminator hairpin are each between 3 and 8 nucleotides long.

19. The composition of any one of claims 16-18, wherein the first stem sequence of the terminator hairpin and the second stem sequence of the terminator hairpin are each four nucleotides long.

20. The composition of any one of claims 16-19, wherein the first stem sequence is 5′-GTTA-3′ and the second stem sequence is 5′-TAAC-3′.

21. The composition of any one of claims 16-19, wherein the first stem sequence is 5′-GATT-3′ and the second stem sequence is 5′-AATC-3′.

22. The composition of any one of claims 16-21, wherein the third stem sequence of the middle hairpin and the fourth stem sequence of the middle hairpin are each between 3 and 20 nucleotides long.

23. The composition of any one of claims 16-22, wherein the third stem sequence of the middle hairpin and the fourth stem sequence of the middle hairpin are each six nucleotides long.

24. The composition of any one of claims 16-23, wherein the third stem sequence is 5′-GAGAAC-3′ and the fourth stem sequence is 5′-GTTCTC-3′.

25. The composition of any one of claims 16-23, wherein the third stem sequence is 5′-CCTGTA-3′ and the fourth stem sequence is 5′-TACAGG-3′.

26. The composition of any one of claims 16-25, wherein the first loop sequence of the terminator hairpin is between 3 and 10 nucleotides long.

27. The composition of any one of claims 16-26, wherein the first loop sequence of the terminator hairpin is four nucleotides long.

28. The composition of any one of claims 16-27, wherein the first loop sequence is 5′-GCAA-3′.

29. The composition of any one of claims 16-28, wherein the second loop sequence of the middle hairpin is between 3 and 15 nucleotides long.

30. The composition of any one of claims 16-29, wherein the second loop sequence of the middle hairpin is four nucleotides long.

31. The composition of any one of claims 16-30, wherein the second loop sequence of the middle hairpin is 5′-ATTA-3′.

32. The composition of any one of claims 16-30, wherein the second loop sequence of the middle hairpin is 5′-CACA-3′.

33. The composition of any one of claims 1-32, wherein the non-extensible oligonucleotide further comprises a mismatch sequence positioned between the binding sequence and the terminator hairpin.

34. The composition of claim 33, wherein the mismatch sequence is between 1 and 100 nucleotides long.

35. The composition of claims 33 or 34, wherein the mismatch sequence is at most 30% identical to the reverse complement of the upstream sequence of the DNA template.

36. The composition of claim 35, wherein the mismatch sequence is unable to hybridize to the upstream sequence of the DNA template.

37. The composition of any one of claims 33-36, wherein the mismatch sequence does not form a non-linear secondary structure.

38. The composition of any one of claims 33-37, wherein the mismatch sequence does not form a hairpin.

39. The composition of claim 37 or 38, wherein the mismatch sequences is between 5 and 20 nucleotides long.

40. The composition of any one of claims 33-36, wherein the mismatch sequence comprises a former subsequence and a latter subsequence, wherein the latter subsequence is the reverse complement of the former subsequence.

41. The composition of claim 40, wherein the former subsequence and the latter subsequence are each at least four nucleotides long.

42. The composition of claim 40 or 41, wherein the former subsequence and the latter subsequence are each six nucleotides long.

43. The composition of any one of claims 40-42, wherein the mismatch sequence comprises a plurality of former subsequences and a plurality of latter subsequences, wherein each former subsequence is the reverse complement of a corresponding latter subsequence.

44. The composition of claim 43, wherein each former subsequence and each latter subsequence is at least four nucleotides long.

45. The composition of claims 43 or 44, wherein the mismatch sequence comprises, from 5′ to 3′, a first subsequence, a second subsequence, a third subsequence, and a fourth subsequence, wherein the first subsequence is the reverse complement of the second subsequence, and wherein the third subsequence is the reverse complement of the fourth subsequence.

46. The composition of claim 45, wherein each of the first subsequence, the second subsequence, the third subsequence, and the fourth subsequence are between four and 15 nucleotides long.

47. The composition of claims 43 or 44, wherein the mismatch sequence comprises, from 5′ to 3′, a first subsequence, a second subsequence, a third subsequence, and a fourth subsequence, wherein the first subsequence is the reverse complement of the fourth subsequence, and wherein the second subsequence is the reverse complement of the third subsequence.

48. The composition of claim 47, wherein each of the first subsequence, the second subsequence, the third subsequence, and the fourth subsequence are between four and 15 nucleotides long.

49. The composition of any one of claims 1-48, wherein the non-extensible oligonucleotide does not comprise an artificial chemical modification or a non-natural DNA nucleotide at its 3′ end.

50. The composition of any one of claims 1-49, wherein the upstream sequence of the DNA template is between 3 and 100 nucleotides long.

51. The composition of any one of claims 1-50, wherein the probe binding sequence of the DNA template is between 10 and 300 nucleotides long.

52. The composition of any one of claims 1-48, wherein the DNA polymerase is a high-fidelity DNA polymerase with 3′ to 5′ exonuclease activity.

53. A composition comprising a DNA template, a DNA polymerase, and a non-extensible oligonucleotide, wherein the DNA template comprises continuously from 5′ to 3′ an upstream sequence and a probe binding sequence, wherein the non-extensible oligonucleotide comprises from 5′ to 3′:
a binding sequence that is at least 70% identical to the reverse complement of the probe binding sequence of the DNA template,
a mismatch sequence comprising:
a first stem sequence, and
a second stem sequence, wherein the second stem sequence is the reverse complement of the first stem sequence, and
a tail sequence that is at most 40% identical to the reverse complement of the upstream sequence of the DNA template.

54. The composition of claim 53, wherein the binding sequence of the non-extensible oligonucleotide is between 10 and 300 nucleotides long.

55. The composition of claim 53 or 54, wherein the mismatch sequence of the non-extensible oligonucleotide is between 10 and 100 nucleotides long.

56. The composition of any one of claims 53-55, wherein the first stem sequence of the mismatch sequence is between 4 and 45 nucleotides long.

57. The composition of any one of claims 53-56, wherein the second stem sequence of the mismatch sequence is between 4 and 45 nucleotides long.

58. The composition of any one of claims 53-57, wherein the mismatch sequence comprises a plurality of first stem sequence and a plurality of second stem sequences, wherein each second stem sequence is the reverse complement of a corresponding first stem sequence.

59. The composition of claim 58, wherein each first stem sequence and each second stem sequence is between four and 45 nucleotides long.

60. The composition of claims 58 or 59, wherein the mismatch sequence comprises, from 5′ to 3′, a first subsequence, a second subsequence, a third subsequence, and a fourth subsequence, wherein the first subsequence is the reverse complement of the second subsequence, and wherein the third subsequence is the reverse complement of the fourth subsequence.

61. The composition of claim 60, wherein each of the first subsequence, the second subsequence, the third subsequence, and the fourth subsequence are between four and 15 nucleotides long.

62. The composition of claims 58 or 59, wherein the mismatch sequence comprises, from 5′ to 3′, a first subsequence, a second subsequence, a third subsequence, and a fourth subsequence, wherein the first subsequence is the reverse complement of the fourth subsequence, and wherein the second subsequence is the reverse complement of the third subsequence.

63. The composition of claim 62, wherein each of the first subsequence, the second subsequence, the third subsequence, and the fourth subsequence are between four and 15 nucleotides long.

64. The composition of any one of claims 53-63, wherein the tail sequence is between 3 and 15 nucleotides long.

65. The composition of any one of claims 53-64, wherein the tail sequence of the non-extensible oligonucleotide is unable to hybridize to the upstream sequence of the DNA template.

66. The composition of any one of claims 53-65, wherein the tail sequence of the non-extensible oligonucleotide does not form a non-linear secondary structure.

67. The composition of any one of claims 53-66, wherein the tail sequence of the non-extensible oligonucleotide does not form a hairpin.

68. The composition of any one of claims 53-67, wherein the non-extensible oligonucleotide does not comprise an artificial chemical modification or a non-natural DNA nucleotide at its 3′ end.

69. The composition of any one of claims 53-68, wherein the upstream sequence of the DNA template is between 3 and 100 nucleotides long.

70. The composition of any one of claims 53-69, wherein the probe binding sequence of the DNA template is between 10 and 300 nucleotides long.

71. The composition of any one of claims 53-70, wherein the DNA polymerase is a high-fidelity DNA polymerase with 3′ to 5′ exonuclease activity.

72. A method for selectively inhibiting a polymerase chain reaction (PCR) amplification of a template DNA having a selected sequence, the method comprising:
(a) mixing a composition of any one of claims 1-71, a forward primer, a reverse primer, and dNTPs under conditions suitable for DNA polymerase activity, wherein the template DNA possibly comprises a target DNA template molecule and possibly comprises a background DNA template molecule; and
(b) subjecting the mixture to at least 7 rounds of thermal cycling.

73. The method of claim 72, wherein each round of thermal cycling comprises holding the mixture at a temperature of at least 78° C. for between 1 second and 30 minutes and then holding the mixing at a temperature of at most 75° C. for between 1 second and 4 hours.

74. The method of claim 72 or 73, wherein the forward primer is between 12 and 60 nucleotides long.

75. The method of any one of claims 71-74, wherein the forward primer is at least 80% identical to the reverse complement of a subsequence of the target DNA template.

76. The method of any one of claims 71-75, wherein the reverse primer is between 12 and 60 nucleotides long.

77. The method of any one of claims 71-76, wherein the reverse primer is at least 80% identical to a subsequence of the target DNA template.

78. The method of any one of claims 71-77, wherein the DNA template optionally comprises a target DNA template.

79. The method of any one of claims 71-78, wherein the DNA template comprises a background DNA template.

80. The method of any one of claims 71-79, wherein the non-extensible oligonucleotide has a binding sequence that is at least 80% homologous to the reverse complement of the probe binding sequence of the background DNA template.

81. The method of any one of claims 71-80, wherein the non-extensible oligonucleotide does not comprise an artificial chemical modification or a non-natural DNA nucleotide at its 3′ end.

82. The method of claim 79 or 80, wherein the background DNA template is a pseudogene.

83. The method of claim 82, wherein the target DNA template is a gene sequence with above 80% homology to the pseudogene.

84. The method of claim 79 or 80, wherein the background DNA template is a wildtype gene sequence.

85. The method of claim 84, wherein the target DNA template is a variant gene sequence with a single nucleotide replacement, a two-nucleotide replacement, an insertion of between 1 and 50 nucleotides, or a deletion of between 1 and 50 nucleotides.

86. The method of any one of claims 71-85, wherein step (a) is performed using the composition of any one of claims 1-52.

87. The method of any one of claims 71-85, wherein step (a) is performed using the composition of any one of claims 53-71.

88. The method of any one of claims 71-87, wherein the binding sequence of the non-extensible oligonucleotide is at least 70% homologous to a 15 nucleotide subsequence of the forward primer.

89. The method of any one of claims 71-88, wherein the mixture of step (a) comprises between 100 pM and 5 µM of the forward primer, between 100 pM and 5 µM of the reverse primer, and between 100 pM and 5 µM of the non-extensible oligonucleotide.

90. The method of any one of claims 71-89, wherein the DNA polymerase is a high-fidelity DNA polymerase with 3′ to 5′ exonuclease activity.

91. The method of any one of claims 71-90, wherein the mixture of step (a) further comprises an intercalating DNA dye or a Taqman probe.

92. The method of claim 91, wherein the quantity or concentration of the target DNA template is determined based on the cycle threshold (Ct) value.

93. The method of any one of claims 71-90, wherein the forward primer further comprises a forward adapter at its 5′ end, and the reverse primer further comprises a reverse adapter at its 5′ end, and the method further comprises (c) performing high-throughput sequencing.

94. The method of any one of claims 71-90, wherein the method further comprises (c) ligating an adapter sequence to the PCR product produced in step (b), and (d) performing high-throughput sequencing.


SEQUENCE LISTING (260 sequences)

SEQ ID NO: 1 - DNA, 47 bp, Artificial sequence
  Synthetic oligonucleotide
actgctgcaggcgccctgtgagaacattagttctcgattgcaaaatc

SEQ ID NO: 2 - DNA, 66 bp, Artificial sequence
  Synthetic oligonucleotide
gactatgtgacaaaatagctaaggatacaggaaatatggagaacattagttctcgattgcaaaatc

SEQ ID NO: 3 - DNA, 66 bp, Artificial sequence
  Synthetic oligonucleotide
catctttatttaacccattagaaaatcctatcagctctgagaacattagttctcgattgcaaaatc

SEQ ID NO: 4 - DNA, 62 bp, Artificial sequence
  Synthetic oligonucleotide
ccgtcataacaaaaacatatttactttctctggcgagaacattagttctcgattgcaaaatc

SEQ ID NO: 5 - DNA, 57 bp, Artificial sequence
  Synthetic oligonucleotide
cttcaatattgcagaagtgttgcaagcctgagaacattagttctcgattgcaaaatc

SEQ ID NO: 6 - DNA, 54 bp, Artificial sequence
  Synthetic oligonucleotide
agggagagaacctcctccctcacagagagaacattagttctcgattgcaaaatc

SEQ ID NO: 7 - DNA, 60 bp, Artificial sequence
  Synthetic oligonucleotide
tcaaattcaggtaccttagagggacagctaaagagaacattagttctcgattgcaaaatc

SEQ ID NO: 8 - DNA, 61 bp, Artificial sequence
  Synthetic oligonucleotide
aatgcaaaactcaatgtatcagtgtgaggatgtgagaacattagttctcgattgcaaaatc

SEQ ID NO: 9 - DNA, 59 bp, Artificial sequence
  Synthetic oligonucleotide
tagcttcagaaacattccagtgtatgtgcaggagaacattagttctcgattgcaaaatc

SEQ ID NO: 10 - DNA, 60 bp, Artificial sequence
  Synthetic oligonucleotide
gttagagcaactttccttgattcccagagtaggagaacattagttctcgattgcaaaatc

SEQ ID NO: 11 - DNA, 58 bp, Artificial sequence
  Synthetic oligonucleotide
ccttgaaaagagggcttaggttttctttgcgagaacattagttctcgattgcaaaatc

SEQ ID NO: 12 - DNA, 65 bp, Artificial sequence
  Synthetic oligonucleotide
cttatgaagtcatggaacaatgcctacttctatatttgagaacattagttctcgattgcaaaatc

SEQ ID NO: 13 - DNA, 66 bp, Artificial sequence
  Synthetic oligonucleotide
ctgagaacttagcattaattaccttttttcatgagaatgagaacattagttctcgattgcaaaatc

SEQ ID NO: 14 - DNA, 62 bp, Artificial sequence
  Synthetic oligonucleotide
agggcattttttacagtgttgaatattgaaactggagaacattagttctcgattgcaaaatc

SEQ ID NO: 15 - DNA, 58 bp, Artificial sequence
  Synthetic oligonucleotide
ctctcatggtatggtgtttttctgtgctccgagaacattagttctcgattgcaaaatc

SEQ ID NO: 16 - DNA, 69 bp, Artificial sequence
  Synthetic oligonucleotide
cagatgaaaattatctgtgcttttttgtaagctgatatattgagaacattagttctcgattgcaaaatc

SEQ ID NO: 17 - DNA, 64 bp, Artificial sequence
  Synthetic oligonucleotide
caatctctgaatctcagaatagtagcctagaaaacggagaacattagttctcgattgcaaaatc

SEQ ID NO: 18 - DNA, 63 bp, Artificial sequence
  Synthetic oligonucleotide
ctcatgagttaacaaggagatgatgtagtgtaaaggagaacattagttctcgattgcaaaatc

SEQ ID NO: 19 - DNA, 56 bp, Artificial sequence
  Synthetic oligonucleotide
caacaaacatgccttctccttctcctgagagaacattagttctcgattgcaaaatc

SEQ ID NO: 20 - DNA, 61 bp, Artificial sequence
  Synthetic oligonucleotide
taaacaccaagacgtggtaaatatttacctggtgagaacattagttctcgattgcaaaatc

SEQ ID NO: 21 - DNA, 58 bp, Artificial sequence
  Synthetic oligonucleotide
caacaaggtcagtattgataagtggttgctgagaacattagttctcgattgcaaaatc

SEQ ID NO: 22 - DNA, 59 bp, Artificial sequence
  Synthetic oligonucleotide
acatgaatcatgtcactattcaatgggatgcgagaacattagttctcgattgcaaaatc

SEQ ID NO: 23 - DNA, 63 bp, Artificial sequence
  Synthetic oligonucleotide
ttttgtttcacatgataaccatatcactggacacagagaacattagttctcgattgcaaaatc

SEQ ID NO: 24 - DNA, 49 bp, Artificial sequence
  Synthetic oligonucleotide
aggatgtgcgacgtgtgcctggagaacattagttctcgattgcaaaatc

SEQ ID NO: 25 - DNA, 52 bp, Artificial sequence
  Synthetic oligonucleotide
ggtccaaagccggaagggcctaaagagaacattagttctcgattgcaaaatc

SEQ ID NO: 26 - DNA, 50 bp, Artificial sequence
  Synthetic oligonucleotide
gcctggaaccgagacgcctcaggagaacattagttctcgattgcaaaatc

SEQ ID NO: 27 - DNA, 52 bp, Artificial sequence
  Synthetic oligonucleotide
tccattggctactcagtctcggctgagaacattagttctcgattgcaaaatc

SEQ ID NO: 28 - DNA, 62 bp, Artificial sequence
  Synthetic oligonucleotide
tcatattgagcttaagagttcagaacactgatgggagaacattagttctcgattgcaaaatc

SEQ ID NO: 29 - DNA, 61 bp, Artificial sequence
  Synthetic oligonucleotide
cataattgcatataacctacacacattctcccagagaacattagttctcgattgcaaaatc

SEQ ID NO: 30 - DNA, 61 bp, Artificial sequence
  Synthetic oligonucleotide
gttatgtgctggaaagagcataaattttggaatgagaacattagttctcgattgcaaaatc

SEQ ID NO: 31 - DNA, 71 bp, Artificial sequence
  Synthetic oligonucleotide
ctcctactgtacatacatattatcttaaggaaaaaatccaaatgagaacattagttctcgattgcaaaatc

SEQ ID NO: 32 - DNA, 71 bp, Artificial sequence
  Synthetic oligonucleotide
tgttcaatgtattaaataatcatcagcatatttttgtattcacgagaacattagttctcgattgcaaaatc

SEQ ID NO: 33 - DNA, 59 bp, Artificial sequence
  Synthetic oligonucleotide
gattggtagaagacactgattgcatcttcaagagaacattagttctcgattgcaaaatc

SEQ ID NO: 34 - DNA, 55 bp, Artificial sequence
  Synthetic oligonucleotide
aaggcacagaacaatcatgcaacttgcgagaacattagttctcgattgcaaaatc

SEQ ID NO: 35 - DNA, 53 bp, Artificial sequence
  Synthetic oligonucleotide
gggactttcttgagggatggcatccgagaacattagttctcgattgcaaaatc

SEQ ID NO: 36 - DNA, 60 bp, Artificial sequence
  Synthetic oligonucleotide
gaggtgatatctcattttggcttctatttgcagagaacattagttctcgattgcaaaatc

SEQ ID NO: 37 - DNA, 52 bp, Artificial sequence
  Synthetic oligonucleotide
tgtcaaacacccatgctcacccttgagaacattagttctcgattgcaaaatc

SEQ ID NO: 38 - DNA, 61 bp, Artificial sequence
  Synthetic oligonucleotide
ggtcctctttaaggtctctacaataaattgccagagaacattagttctcgattgcaaaatc

SEQ ID NO: 39 - DNA, 61 bp, Artificial sequence
  Synthetic oligonucleotide
gacataatgcttttggttggactttcaaaaagggagaacattagttctcgattgcaaaatc

SEQ ID NO: 40 - DNA, 60 bp, Artificial sequence
  Synthetic oligonucleotide
gcaaggttcaaatcattctctcctatctcatcgagaacattagttctcgattgcaaaatc

SEQ ID NO: 41 - DNA, 63 bp, Artificial sequence
  Synthetic oligonucleotide
gctagagagataattgagtgtcatcagaactagatgagaacattagttctcgattgcaaaatc

SEQ ID NO: 42 - DNA, 64 bp, Artificial sequence
  Synthetic oligonucleotide
atgaggagtaattgaaatcattaatacccacaaacagagaacattagttctcgattgcaaaatc

SEQ ID NO: 43 - DNA, 60 bp, Artificial sequence
  Synthetic oligonucleotide
aactcagaccaatttggccatagattattagcgagaacattagttctcgattgcaaaatc

SEQ ID NO: 44 - DNA, 67 bp, Artificial sequence
  Synthetic oligonucleotide
acaaaaccctataaggaagatgtcattacccatattttagagaacattagttctcgattgcaaaatc

SEQ ID NO: 45 - DNA, 62 bp, Artificial sequence
  Synthetic oligonucleotide
acccagctttatacattcacaaagatatggtttggagaacattagttctcgattgcaaaatc

SEQ ID NO: 46 - DNA, 57 bp, Artificial sequence
  Synthetic oligonucleotide
acagtggaacagctctctccttcttttttgagaacattagttctcgattgcaaaatc

SEQ ID NO: 47 - DNA, 71 bp, Artificial sequence
  Synthetic oligonucleotide
attagaataactactattaaaaaaaccccacaaaataactcttgagaacattagttctcgattgcaaaatc

SEQ ID NO: 48 - DNA, 65 bp, Artificial sequence
  Synthetic oligonucleotide
tttgggaattaaaagccaatagattagctgaaaattcgagaacattagttctcgattgcaaaatc

SEQ ID NO: 49 - DNA, 60 bp, Artificial sequence
  Synthetic oligonucleotide
acacctttacatgaaggctttgaagtactcttgagaacattagttctcgattgcaaaatc

SEQ ID NO: 50 - DNA, 70 bp, Artificial sequence
  Synthetic oligonucleotide
gtgcattatgggtaagaatgttcatttattatttcacttatagagaacattagttctcgattgcaaaatc

SEQ ID NO: 51 - DNA, 57 bp, Artificial sequence
  Synthetic oligonucleotide
gaagtcgtagctattcggcaaaggaaatggagaacattagttctcgattgcaaaatc

SEQ ID NO: 52 - DNA, 54 bp, Artificial sequence
  Synthetic oligonucleotide
tgccccataggcagtgtttggtgaaggagaacattagttctcgattgcaaaatc

SEQ ID NO: 53 - DNA, 57 bp, Artificial sequence
  Synthetic oligonucleotide
taccccatgtgtatcaaatggtcagcaaggagaacattagttctcgattgcaaaatc

SEQ ID NO: 54 - DNA, 52 bp, Artificial sequence
  Synthetic oligonucleotide
ctgtgagttgggagcaaaggagcagagaacattagttctcgattgcaaaatc

SEQ ID NO: 55 - DNA, 55 bp, Artificial sequence
  Synthetic oligonucleotide
ctctggagacgggggatgttaagttgagagaacattagttctcgattgcaaaatc

SEQ ID NO: 56 - DNA, 54 bp, Artificial sequence
  Synthetic oligonucleotide
tctggtgattgagaaagcgttccagagagaacattagttctcgattgcaaaatc

SEQ ID NO: 57 - DNA, 60 bp, Artificial sequence
  Synthetic oligonucleotide
attggattaactttggtggaacctacttcgatgagaacattagttctcgattgcaaaatc

SEQ ID NO: 58 - DNA, 57 bp, Artificial sequence
  Synthetic oligonucleotide
ctcccttctttcatccctacatcatgtccgagaacattagttctcgattgcaaaatc

SEQ ID NO: 59 - DNA, 51 bp, Artificial sequence
  Synthetic oligonucleotide
cggacttggctggggtagagcttgagaacattagttctcgattgcaaaatc

SEQ ID NO: 60 - DNA, 55 bp, Artificial sequence
  Synthetic oligonucleotide
gagctaagtaccaggtatgatgctcgcgagaacattagttctcgattgcaaaatc

SEQ ID NO: 61 - DNA, 57 bp, Artificial sequence
  Synthetic oligonucleotide
agggaatgctctagacaaaacactgttccgagaacattagttctcgattgcaaaatc

SEQ ID NO: 62 - DNA, 56 bp, Artificial sequence
  Synthetic oligonucleotide
tgctttgtgctagctcaaagactcacatgagaacattagttctcgattgcaaaatc

SEQ ID NO: 63 - DNA, 62 bp, Artificial sequence
  Synthetic oligonucleotide
aattctggatcaaattaaatatgtcgcattctccgagaacattagttctcgattgcaaaatc

SEQ ID NO: 64 - DNA, 63 bp, Artificial sequence
  Synthetic oligonucleotide
tgtactttctttttagccataagatgatttcccatgagaacattagttctcgattgcaaaatc

SEQ ID NO: 65 - DNA, 52 bp, Artificial sequence
  Synthetic oligonucleotide
gcttgctttcccacaccactacctgagaacattagttctcgattgcaaaatc

SEQ ID NO: 66 - DNA, 54 bp, Artificial sequence
  Synthetic oligonucleotide
ggtcaagtctgaggctgttgagcttagagaacattagttctcgattgcaaaatc

SEQ ID NO: 67 - DNA, 53 bp, Artificial sequence
  Synthetic oligonucleotide
ttcaggacgtgaaagcacgagaacggagaacattagttctcgattgcaaaatc

SEQ ID NO: 68 - DNA, 59 bp, Artificial sequence
  Synthetic oligonucleotide
atgtacaatttcaactggagtttccattgcagagaacattagttctcgattgcaaaatc

SEQ ID NO: 69 - DNA, 54 bp, Artificial sequence
  Synthetic oligonucleotide
aaatagtgagaacgagcagctgcagggagaacattagttctcgattgcaaaatc

SEQ ID NO: 70 - DNA, 65 bp, Artificial sequence
  Synthetic oligonucleotide
aagaatataaaatgttagagaaccacatacaacgagcgagaacattagttctcgattgcaaaatc

SEQ ID NO: 71 - DNA, 54 bp, Artificial sequence
  Synthetic oligonucleotide
aaccccagaacactagcagctaaggggagaacattagttctcgattgcaaaatc

SEQ ID NO: 72 - DNA, 69 bp, Artificial sequence
  Synthetic oligonucleotide
ttttattagttgtgtaatccagttacttaactttaaaagccgagaacattagttctcgattgcaaaatc

SEQ ID NO: 73 - DNA, 54 bp, Artificial sequence
  Synthetic oligonucleotide
gttctgaaaagagcctccactcctgtgagaacattagttctcgattgcaaaatc

SEQ ID NO: 74 - DNA, 54 bp, Artificial sequence
  Synthetic oligonucleotide
cctccactccaccatggcacctattagagaacattagttctcgattgcaaaatc

SEQ ID NO: 75 - DNA, 69 bp, Artificial sequence
  Synthetic oligonucleotide
gtataccacttaggctatagttattctaaactttgataaacgagaacattagttctcgattgcaaaatc

SEQ ID NO: 76 - DNA, 63 bp, Artificial sequence
  Synthetic oligonucleotide
aggaatcattacaggaaaacatcgtttaaattggagagaacattagttctcgattgcaaaatc

SEQ ID NO: 77 - DNA, 61 bp, Artificial sequence
  Synthetic oligonucleotide
ccatggtatattgtaagttgtaggtacatacccgagaacattagttctcgattgcaaaatc

SEQ ID NO: 78 - DNA, 61 bp, Artificial sequence
  Synthetic oligonucleotide
tttgatttgaataaaccagagaactcttctgaggagaacattagttctcgattgcaaaatc

SEQ ID NO: 79 - DNA, 58 bp, Artificial sequence
  Synthetic oligonucleotide
tgttgctatcttgcttttagcatttagtgcgagaacattagttctcgattgcaaaatc

SEQ ID NO: 80 - DNA, 53 bp, Artificial sequence
  Synthetic oligonucleotide
tcagcgttgagtaataccgtctgccgagaacattagttctcgattgcaaaatc

SEQ ID NO: 81 - DNA, 63 bp, Artificial sequence
  Synthetic oligonucleotide
gctagagagataattgagtgtcatcagaactagatgagaacattagttctcgttagcaataac

SEQ ID NO: 82 - DNA, 66 bp, Artificial sequence
  Synthetic oligonucleotide
ctgagaacttagcattaattaccttttttcatgagaatcctgtacacatacagggttagcaataac

SEQ ID NO: 83 - DNA, 47 bp, Artificial sequence
  Synthetic oligonucleotide
actgctgcaggcgccctgtcctgtacacatacagggttagcaataac

SEQ ID NO: 84 - DNA, 20 bp, Artificial sequence
  Synthetic oligonucleotide
accaatgggagtcactgctg

SEQ ID NO: 85 - DNA, 28 bp, Artificial sequence
  Synthetic oligonucleotide
gaggggtattagaagaatgactatgtga

SEQ ID NO: 86 - DNA, 28 bp, Artificial sequence
  Synthetic oligonucleotide
gctcttcctctcacatctttatttaacc

SEQ ID NO: 87 - DNA, 28 bp, Artificial sequence
  Synthetic oligonucleotide
cagagtaaaatttactgctccgtcataa

SEQ ID NO: 88 - DNA, 26 bp, Artificial sequence
  Synthetic oligonucleotide
ggattccctaagctcttcaatattgc

SEQ ID NO: 89 - DNA, 24 bp, Artificial sequence
  Synthetic oligonucleotide
cctcatctgtaaagcagggagaga

SEQ ID NO: 90 - DNA, 25 bp, Artificial sequence
  Synthetic oligonucleotide
acttctgccaacattcaaattcagg

SEQ ID NO: 91 - DNA, 23 bp, Artificial sequence
  Synthetic oligonucleotide
ggatgggactccaatgcaaaact

SEQ ID NO: 92 - DNA, 28 bp, Artificial sequence
  Synthetic oligonucleotide
catcttgctcttcatagatagcttcaga

SEQ ID NO: 93 - DNA, 27 bp, Artificial sequence
  Synthetic oligonucleotide
cctgaatgtcagttttgttagagcaac

SEQ ID NO: 94 - DNA, 27 bp, Artificial sequence
  Synthetic oligonucleotide
cagacttaatcaaagcccttgaaaaga

SEQ ID NO: 95 - DNA, 27 bp, Artificial sequence
  Synthetic oligonucleotide
cctcccatagtgattcttatgaagtca

SEQ ID NO: 96 - DNA, 23 bp, Artificial sequence
  Synthetic oligonucleotide
aatgctttgcttgctgagaactt

SEQ ID NO: 97 - DNA, 25 bp, Artificial sequence
  Synthetic oligonucleotide
aatgggaaaacacattttaagggca

SEQ ID NO: 98 - DNA, 23 bp, Artificial sequence
  Synthetic oligonucleotide
ccacactctgcctctcatggtat

SEQ ID NO: 99 - DNA, 31 bp, Artificial sequence
  Synthetic oligonucleotide
acttaagaggtcaacacagatgaaaattatc

SEQ ID NO: 100 - DNA, 24 bp, Artificial sequence
  Synthetic oligonucleotide
tccgcaaaacctacaatctctgaa

SEQ ID NO: 101 - DNA, 25 bp, Artificial sequence
  Synthetic oligonucleotide
tcagatgctttaggctcatgagtta

SEQ ID NO: 102 - DNA, 24 bp, Artificial sequence
  Synthetic oligonucleotide
ctccttggaatcaccaacaaacat

SEQ ID NO: 103 - DNA, 32 bp, Artificial sequence
  Synthetic oligonucleotide
cttgtatatagacggtaaaataaacaccaaga

SEQ ID NO: 104 - DNA, 24 bp, Artificial sequence
  Synthetic oligonucleotide
tgaagcagatgttgaacaacaagg

SEQ ID NO: 105 - DNA, 23 bp, Artificial sequence
  Synthetic oligonucleotide
ttcctgcttccagacatgaatca

SEQ ID NO: 106 - DNA, 24 bp, Artificial sequence
  Synthetic oligonucleotide
acccaggtgagttttgtttcacat

SEQ ID NO: 107 - DNA, 20 bp, Artificial sequence
  Synthetic oligonucleotide
ctctctgcctgcaggatgtg

SEQ ID NO: 108 - DNA, 23 bp, Artificial sequence
  Synthetic oligonucleotide
cacttcctccagaaggtccaaag

SEQ ID NO: 109 - DNA, 22 bp, Artificial sequence
  Synthetic oligonucleotide
actaagagtgcagagcctggaa

SEQ ID NO: 110 - DNA, 19 bp, Artificial sequence
  Synthetic oligonucleotide
gcgttgtgctgtccattgg

SEQ ID NO: 111 - DNA, 30 bp, Artificial sequence
  Synthetic oligonucleotide
gggttaaaatcttttgctttcatattgagc

SEQ ID NO: 112 - DNA, 28 bp, Artificial sequence
  Synthetic oligonucleotide
cccgttatataagaggacataattgcat

SEQ ID NO: 113 - DNA, 26 bp, Artificial sequence
  Synthetic oligonucleotide
cactttatcagacacagttatgtgct

SEQ ID NO: 114 - DNA, 25 bp, Artificial sequence
  Synthetic oligonucleotide
gccctataggttttcctcctactgt

SEQ ID NO: 115 - DNA, 26 bp, Artificial sequence
  Synthetic oligonucleotide
ctatctgcaggattgtgttcaatgta

SEQ ID NO: 116 - DNA, 25 bp, Artificial sequence
  Synthetic oligonucleotide
ctctctagagtgcagattggtagaa

SEQ ID NO: 117 - DNA, 30 bp, Artificial sequence
  Synthetic oligonucleotide
caaagttgataaattaaaggactaaggcac

SEQ ID NO: 118 - DNA, 21 bp, Artificial sequence
  Synthetic oligonucleotide
ctgacctaaggcatgggactt

SEQ ID NO: 119 - DNA, 23 bp, Artificial sequence
  Synthetic oligonucleotide
cattctgtctgggatgaggtgat

SEQ ID NO: 120 - DNA, 28 bp, Artificial sequence
  Synthetic oligonucleotide
tggaatcaaacatactatgtgtcaaaca

SEQ ID NO: 121 - DNA, 25 bp, Artificial sequence
  Synthetic oligonucleotide
gcgaagtcatttcggtcctctttaa

SEQ ID NO: 122 - DNA, 24 bp, Artificial sequence
  Synthetic oligonucleotide
cctctgattcccagacataatgct

SEQ ID NO: 123 - DNA, 21 bp, Artificial sequence
  Synthetic oligonucleotide
tgaaagacgtcacagcaaggt

SEQ ID NO: 124 - DNA, 24 bp, Artificial sequence
  Synthetic oligonucleotide
tgtaggagagattgggctagagag

SEQ ID NO: 125 - DNA, 28 bp, Artificial sequence
  Synthetic oligonucleotide
acagaaaccaattacctatgaggagtaa

SEQ ID NO: 126 - DNA, 23 bp, Artificial sequence
  Synthetic oligonucleotide
gaaagtggcacagaaactcagac

SEQ ID NO: 127 - DNA, 31 bp, Artificial sequence
  Synthetic oligonucleotide
gcattagatcatttaacacacaaaaccctat

SEQ ID NO: 128 - DNA, 20 bp, Artificial sequence
  Synthetic oligonucleotide
tgctcctaaaagcacccagc

SEQ ID NO: 129 - DNA, 30 bp, Artificial sequence
  Synthetic oligonucleotide
ctaacttcctaactaaaactttacagtgga

SEQ ID NO: 130 - DNA, 30 bp, Artificial sequence
  Synthetic oligonucleotide
ggatttcacacccattagaataactactat

SEQ ID NO: 131 - DNA, 28 bp, Artificial sequence
  Synthetic oligonucleotide
cctctagaaaaaatggagatttgggaat

SEQ ID NO: 132 - DNA, 28 bp, Artificial sequence
  Synthetic oligonucleotide
ggacttttttgctttttgacacctttac

SEQ ID NO: 133 - DNA, 31 bp, Artificial sequence
  Synthetic oligonucleotide
acgtatttctaactatagtgagtgcattatg

SEQ ID NO: 134 - DNA, 25 bp, Artificial sequence
  Synthetic oligonucleotide
acatgtccaaagagagaagtcgtag

SEQ ID NO: 135 - DNA, 24 bp, Artificial sequence
  Synthetic oligonucleotide
gcacgtagatgaaattgccccata

SEQ ID NO: 136 - DNA, 20 bp, Artificial sequence
  Synthetic oligonucleotide
gccccaaaggttaccccatg

SEQ ID NO: 137 - DNA, 21 bp, Artificial sequence
  Synthetic oligonucleotide
gtagccgcttctctgtgagtt

SEQ ID NO: 138 - DNA, 28 bp, Artificial sequence
  Synthetic oligonucleotide
ggaaaatatgtctaaaaaggctctggag

SEQ ID NO: 139 - DNA, 26 bp, Artificial sequence
  Synthetic oligonucleotide
gtttgttctaaggttcatctggtgat

SEQ ID NO: 140 - DNA, 26 bp, Artificial sequence
  Synthetic oligonucleotide
gtgggcttacatgattggattaactt

SEQ ID NO: 141 - DNA, 23 bp, Artificial sequence
  Synthetic oligonucleotide
tgtcccactttttacctcccttc

SEQ ID NO: 142 - DNA, 20 bp, Artificial sequence
  Synthetic oligonucleotide
gggcttcggaatcggacttg

SEQ ID NO: 143 - DNA, 23 bp, Artificial sequence
  Synthetic oligonucleotide
tgtgctacgacagagctaagtac

SEQ ID NO: 144 - DNA, 19 bp, Artificial sequence
  Synthetic oligonucleotide
ccccggatgtcagggaatg

SEQ ID NO: 145 - DNA, 19 bp, Artificial sequence
  Synthetic oligonucleotide
ccaggcaccactgctttgt

SEQ ID NO: 146 - DNA, 30 bp, Artificial sequence
  Synthetic oligonucleotide
gcagaaaaaaatgatatctgaattctggat

SEQ ID NO: 147 - DNA, 31 bp, Artificial sequence
  Synthetic oligonucleotide
cctttttcactgttatgaaatgtactttctt

SEQ ID NO: 148 - DNA, 17 bp, Artificial sequence
  Synthetic oligonucleotide
aggatgctggggcttgc

SEQ ID NO: 149 - DNA, 22 bp, Artificial sequence
  Synthetic oligonucleotide
gtaaagtgcatggggtcaagtc

SEQ ID NO: 150 - DNA, 29 bp, Artificial sequence
  Synthetic oligonucleotide
tgagaagtctaacaagttaaattcaggac

SEQ ID NO: 151 - DNA, 31 bp, Artificial sequence
  Synthetic oligonucleotide
ggtagaatgttagtgactatgtacaatttca

SEQ ID NO: 152 - DNA, 28 bp, Artificial sequence
  Synthetic oligonucleotide
accttgtcaagaacctaaatagtgagaa

SEQ ID NO: 153 - DNA, 30 bp, Artificial sequence
  Synthetic oligonucleotide
cgtgggctagtcaagaatataaaatgttag

SEQ ID NO: 154 - DNA, 37 bp, Artificial sequence
  Synthetic oligonucleotide
cctatatagactaatttacttaaacatttaaacccca

SEQ ID NO: 155 - DNA, 29 bp, Artificial sequence
  Synthetic oligonucleotide
ggttcaactctcagttttattagttgtgt

SEQ ID NO: 156 - DNA, 23 bp, Artificial sequence
  Synthetic oligonucleotide
agcatcgtgaggttctgaaaaga

SEQ ID NO: 157 - DNA, 23 bp, Artificial sequence
  Synthetic oligonucleotide
cctaacaccagttcttcctccac

SEQ ID NO: 158 - DNA, 24 bp, Artificial sequence
  Synthetic oligonucleotide
tgtgtggctcagtataccacttag

SEQ ID NO: 159 - DNA, 31 bp, Artificial sequence
  Synthetic oligonucleotide
gaaaaaaaagggtctcattaggaatcattac

SEQ ID NO: 160 - DNA, 28 bp, Artificial sequence
  Synthetic oligonucleotide
gtttttatatgttagtgtccccatggta

SEQ ID NO: 161 - DNA, 24 bp, Artificial sequence
  Synthetic oligonucleotide
agtgatcagaaggctttgatttga

SEQ ID NO: 162 - DNA, 28 bp, Artificial sequence
  Synthetic oligonucleotide
gcacatcatacattatttctgttgctat

SEQ ID NO: 163 - DNA, 21 bp, Artificial sequence
  Synthetic oligonucleotide
gaaatattgctggggtcagcg

SEQ ID NO: 164 - DNA, 23 bp, Artificial sequence
  Synthetic oligonucleotide
taagtggaaagaactggggtgtc

SEQ ID NO: 165 - DNA, 28 bp, Artificial sequence
  Synthetic oligonucleotide
acatggttagatattagcctgacctatg

SEQ ID NO: 166 - DNA, 23 bp, Artificial sequence
  Synthetic oligonucleotide
gcagccaagtgtgaaagtattga

SEQ ID NO: 167 - DNA, 25 bp, Artificial sequence
  Synthetic oligonucleotide
tgatgttgagttgagacaggttaca

SEQ ID NO: 168 - DNA, 28 bp, Artificial sequence
  Synthetic oligonucleotide
aaatgtagttctattatggtcagcacac

SEQ ID NO: 169 - DNA, 23 bp, Artificial sequence
  Synthetic oligonucleotide
agtatccccaaaaggttgcagat

SEQ ID NO: 170 - DNA, 18 bp, Artificial sequence
  Synthetic oligonucleotide
gtgcaagctggaggcact

SEQ ID NO: 171 - DNA, 29 bp, Artificial sequence
  Synthetic oligonucleotide
acaggaaaagaaactaaaattgtaccctt

SEQ ID NO: 172 - DNA, 23 bp, Artificial sequence
  Synthetic oligonucleotide
gaagccagatctcaaagtgtcct

SEQ ID NO: 173 - DNA, 27 bp, Artificial sequence
  Synthetic oligonucleotide
gttattgggaatgctatgaaagagaca

SEQ ID NO: 174 - DNA, 29 bp, Artificial sequence
  Synthetic oligonucleotide
agaactcatttccttatagctgaagaact

SEQ ID NO: 175 - DNA, 24 bp, Artificial sequence
  Synthetic oligonucleotide
gcagacacttaggatgtttccagt

SEQ ID NO: 176 - DNA, 25 bp, Artificial sequence
  Synthetic oligonucleotide
gagccttagttcctccatcagtaaa

SEQ ID NO: 177 - DNA, 27 bp, Artificial sequence
  Synthetic oligonucleotide
aaatttacgttggtaattgggtcttgt

SEQ ID NO: 178 - DNA, 22 bp, Artificial sequence
  Synthetic oligonucleotide
cacagaggtgacagaacacagt

SEQ ID NO: 179 - DNA, 24 bp, Artificial sequence
  Synthetic oligonucleotide
tagttattcatggtgggaaggcaa

SEQ ID NO: 180 - DNA, 31 bp, Artificial sequence
  Synthetic oligonucleotide
aaaagataatgttcttgtttatatgcccttg

SEQ ID NO: 181 - DNA, 27 bp, Artificial sequence
  Synthetic oligonucleotide
tacagcaactcacaaactaatgactct

SEQ ID NO: 182 - DNA, 19 bp, Artificial sequence
  Synthetic oligonucleotide
ggctgcgatgagacaggaa

SEQ ID NO: 183 - DNA, 23 bp, Artificial sequence
  Synthetic oligonucleotide
aggcaccagaagtcatcagaatg

SEQ ID NO: 184 - DNA, 22 bp, Artificial sequence
  Synthetic oligonucleotide
gaccaagcttttatgcaccaca

SEQ ID NO: 185 - DNA, 31 bp, Artificial sequence
  Synthetic oligonucleotide
tgaaagatagcaatagatacataaaacacca

SEQ ID NO: 186 - DNA, 26 bp, Artificial sequence
  Synthetic oligonucleotide
tgaaaccacatacacacaaattcact

SEQ ID NO: 187 - DNA, 23 bp, Artificial sequence
  Synthetic oligonucleotide
cttcctgctgtgcatctgtaagt

SEQ ID NO: 188 - DNA, 24 bp, Artificial sequence
  Synthetic oligonucleotide
aaaaagaagaaacggaaggcagag

SEQ ID NO: 189 - DNA, 22 bp, Artificial sequence
  Synthetic oligonucleotide
tgctgccccaccctttattaac

SEQ ID NO: 190 - DNA, 23 bp, Artificial sequence
  Synthetic oligonucleotide
cctattggaagaacctgccagaa

SEQ ID NO: 191 - DNA, 20 bp, Artificial sequence
  Synthetic oligonucleotide
tgcaaaatgaagcacagccc

SEQ ID NO: 192 - DNA, 28 bp, Artificial sequence
  Synthetic oligonucleotide
gcagatggaaaatacttgggaaaaaaat

SEQ ID NO: 193 - DNA, 33 bp, Artificial sequence
  Synthetic oligonucleotide
gcaaaaattactataccgactttaataacgaaa

SEQ ID NO: 194 - DNA, 23 bp, Artificial sequence
  Synthetic oligonucleotide
actcattgtaggctgaaccttgg

SEQ ID NO: 195 - DNA, 24 bp, Artificial sequence
  Synthetic oligonucleotide
tgtattgagcatttagcacatgcc

SEQ ID NO: 196 - DNA, 24 bp, Artificial sequence
  Synthetic oligonucleotide
caatttccaagacagaagcactcc

SEQ ID NO: 197 - DNA, 21 bp, Artificial sequence
  Synthetic oligonucleotide
acttactgagcacatggcctg

SEQ ID NO: 198 - DNA, 21 bp, Artificial sequence
  Synthetic oligonucleotide
ggagagggtgagaagttgcac

SEQ ID NO: 199 - DNA, 28 bp, Artificial sequence
  Synthetic oligonucleotide
ggcaaagacatttttccaaggaagatat

SEQ ID NO: 200 - DNA, 18 bp, Artificial sequence
  Synthetic oligonucleotide
cactgccagcttgtgcct

SEQ ID NO: 201 - DNA, 26 bp, Artificial sequence
  Synthetic oligonucleotide
gccctaaatcctaaatgaaattggca

SEQ ID NO: 202 - DNA, 24 bp, Artificial sequence
  Synthetic oligonucleotide
agaggagaaatagatgtagctgcc

SEQ ID NO: 203 - DNA, 28 bp, Artificial sequence
  Synthetic oligonucleotide
aatccagtgacattctttaaactgtctt

SEQ ID NO: 204 - DNA, 22 bp, Artificial sequence
  Synthetic oligonucleotide
gctgagctgtcacatcacttca

SEQ ID NO: 205 - DNA, 25 bp, Artificial sequence
  Synthetic oligonucleotide
gctgtgtagcttggcaaattaacta

SEQ ID NO: 206 - DNA, 22 bp, Artificial sequence
  Synthetic oligonucleotide
gcactcttgggtaacaggcttt

SEQ ID NO: 207 - DNA, 16 bp, Artificial sequence
  Synthetic oligonucleotide
ccatgcccagcctggc

SEQ ID NO: 208 - DNA, 26 bp, Artificial sequence
  Synthetic oligonucleotide
tggctcctcataagttatgcagattt

SEQ ID NO: 209 - DNA, 28 bp, Artificial sequence
  Synthetic oligonucleotide
cagtaggattggctttatcaaagagatc

SEQ ID NO: 210 - DNA, 28 bp, Artificial sequence
  Synthetic oligonucleotide
accataatgttttccatagaagatgcac

SEQ ID NO: 211 - DNA, 32 bp, Artificial sequence
  Synthetic oligonucleotide
ggttctgtactgaagtaaaaatctcatactat

SEQ ID NO: 212 - DNA, 28 bp, Artificial sequence
  Synthetic oligonucleotide
ggcaaagaaacatggcagaaatatcata

SEQ ID NO: 213 - DNA, 25 bp, Artificial sequence
  Synthetic oligonucleotide
cctttggcattttggtcaagattgt

SEQ ID NO: 214 - DNA, 25 bp, Artificial sequence
  Synthetic oligonucleotide
gggggaaaatggtttcttaggatga

SEQ ID NO: 215 - DNA, 31 bp, Artificial sequence
  Synthetic oligonucleotide
ctcccatttttctaagacatttttttttctc

SEQ ID NO: 216 - DNA, 16 bp, Artificial sequence
  Synthetic oligonucleotide
agcatgccgcccttgg

SEQ ID NO: 217 - DNA, 27 bp, Artificial sequence
  Synthetic oligonucleotide
tcacaggtcaaaattatgagttcttcg

SEQ ID NO: 218 - DNA, 22 bp, Artificial sequence
  Synthetic oligonucleotide
tgagagtgtgcaagtcacttgt

SEQ ID NO: 219 - DNA, 20 bp, Artificial sequence
  Synthetic oligonucleotide
gcaggcagcatgtatcccag

SEQ ID NO: 220 - DNA, 31 bp, Artificial sequence
  Synthetic oligonucleotide
gtttaatggacagtagatgctaaattctaga

SEQ ID NO: 221 - DNA, 20 bp, Artificial sequence
  Synthetic oligonucleotide
cgccatagttagccgcttcc

SEQ ID NO: 222 - DNA, 20 bp, Artificial sequence
  Synthetic oligonucleotide
tgagcctcggtctctacctg

SEQ ID NO: 223 - DNA, 20 bp, Artificial sequence
  Synthetic oligonucleotide
cctttaaggcccagcaactg

SEQ ID NO: 224 - DNA, 23 bp, Artificial sequence
  Synthetic oligonucleotide
gggtgacctttcccttttgatga

SEQ ID NO: 225 - DNA, 26 bp, Artificial sequence
  Synthetic oligonucleotide
tgtgtgtgaaagcactttataaacca

SEQ ID NO: 226 - DNA, 34 bp, Artificial sequence
  Synthetic oligonucleotide
ctatcctcagaattttccattgatactagaaata

SEQ ID NO: 227 - DNA, 23 bp, Artificial sequence
  Synthetic oligonucleotide
gagtgtctcccaaacaaggatca

SEQ ID NO: 228 - DNA, 22 bp, Artificial sequence
  Synthetic oligonucleotide
acagccatcagatatccagcag

SEQ ID NO: 229 - DNA, 25 bp, Artificial sequence
  Synthetic oligonucleotide
acttcgagaattgactctaagtggt

SEQ ID NO: 230 - DNA, 29 bp, Artificial sequence
  Synthetic oligonucleotide
aatttagcttccttgaggatagaagtaac

SEQ ID NO: 231 - DNA, 17 bp, Artificial sequence
  Synthetic oligonucleotide
cccggccacccatacag

SEQ ID NO: 232 - DNA, 31 bp, Artificial sequence
  Synthetic oligonucleotide
gaaaactaccttaaactatgtgagaaagaac

SEQ ID NO: 233 - DNA, 28 bp, Artificial sequence
  Synthetic oligonucleotide
accctcactaatctttttctgtttgttt

SEQ ID NO: 234 - DNA, 24 bp, Artificial sequence
  Synthetic oligonucleotide
gtttttctcccagctgtaaaagca

SEQ ID NO: 235 - DNA, 31 bp, Artificial sequence
  Synthetic oligonucleotide
gctttagtttctttgcatattttctgcaata

SEQ ID NO: 236 - DNA, 24 bp, Artificial sequence
  Synthetic oligonucleotide
agctgatctgcaaggtctatttga

SEQ ID NO: 237 - DNA, 21 bp, Artificial sequence
  Synthetic oligonucleotide
tgggctcaagtgatccaccta

SEQ ID NO: 238 - DNA, 25 bp, Artificial sequence
  Synthetic oligonucleotide
gtaaagagaagggctaccaggatta

SEQ ID NO: 239 - DNA, 23 bp, Artificial sequence
  Synthetic oligonucleotide
ccctatgcctgggatacttcctt

SEQ ID NO: 240 - DNA, 22 bp, Artificial sequence
  Synthetic oligonucleotide
agaccgaacttgttgcgaatca

SEQ ID NO: 241 - DNA, 29 bp, Artificial sequence
  Synthetic oligonucleotide
tcttttatccagttgcctctattttacac

SEQ ID NO: 242 - DNA, 27 bp, Artificial sequence
  Synthetic oligonucleotide
ttcaaaaacccattcatacaaggtcag

SEQ ID NO: 243 - DNA, 19 bp, Artificial sequence
  Synthetic oligonucleotide
aggagagggaggagcatgg

SEQ ID NO: 244 - DNA, 12 bp, Artificial sequence
  Synthetic oligonucleotide
tctcgcaagaga

SEQ ID NO: 245 - DNA, 12 bp, Artificial sequence
  Synthetic oligonucleotide
gttcgcaagaac

SEQ ID NO: 246 - DNA, 60 bp, Artificial sequence
  Synthetic oligonucleotide
gactatgtgacaaaatagctaaggatacaggaaatatggaacattagttctcgcaagaga

SEQ ID NO: 247 - DNA, 60 bp, Artificial sequence
  Synthetic oligonucleotide
gactatgtgacaaaatagctaaggatacaggaaatatggaacattagtactcgcaagagt

SEQ ID NO: 248 - DNA, 60 bp, Artificial sequence
  Synthetic oligonucleotide
gactatgtgacaaaatagctaaggatacaggaaatatggaacattagtgttcgcaagaac

SEQ ID NO: 249 - DNA, 87 bp, Artificial sequence
  Synthetic oligonucleotide
gactatgtgacaaaatagctaaggatacaggaaatatgcgtaagtcatcttacgtgagagaacattagttctccttctccgttgaga

SEQ ID NO: 250 - DNA, 87 bp, Artificial sequence
  Synthetic oligonucleotide
gactatgtgacaaaatagctaaggatacaggaaatatggagaacattacctgtatgatacaggcacagttctccttctccgttgaga

SEQ ID NO: 251 - DNA, 32 bp, Artificial sequence
  Synthetic oligonucleotide
taaacaccaagacgtggtaaatatttacctgg

SEQ ID NO: 252 - DNA, 41 bp, Artificial sequence
  Synthetic oligonucleotide
gactatgtgacaaaatagctaaggatacaggaaatatttaa

SEQ ID NO: 253 - DNA, 39 bp, Artificial sequence
  Synthetic oligonucleotide
actgctgcaggcgccctgtacactttaactccgcaagga

SEQ ID NO: 254 - DNA, 45 bp, Artificial sequence
  Synthetic oligonucleotide
actgctgcaggcgccctgtacactttaactcaatcgcaagattga

SEQ ID NO: 255 - DNA, 69 bp, Artificial sequence
  Synthetic oligonucleotide
actgctgcaggcgccctgtctgagaacattagttctcagcctgagaacattagttctcagtaccccact

SEQ ID NO: 256 - DNA, 100 bp, Artificial sequence
  Synthetic oligonucleotide
gactatgtgacaaaatagctaaggatacaggaaatatacgcaggctgagaacattagttctcagtgaccctaattatagggtcacctgcgttggcaagag

SEQ ID NO: 257 - DNA, 62 bp, Artificial sequence
  Synthetic oligonucleotide
actgctgcaggcgccctgtcgtaagtcattgagagaacattagttctccttatagcaagaga

SEQ ID NO: 258 - DNA, 28 bp, Artificial sequence
  Synthetic oligonucleotide
gagaacattagttctcgttagcaataac

SEQ ID NO: 259 - DNA, 28 bp, Artificial sequence
  Synthetic oligonucleotide
gagaacattagttctcgattgcaaaatc

SEQ ID NO: 260 - DNA, 28 bp, Artificial sequence
  Synthetic oligonucleotide
cctgtacacatacagggttagcaataac